(19) United States
(12) Patent Application Publication
(10) Pub. No.: US 2011/0166196 A1
(43) Pub. Date: Jul. 7, 2011

(54) TREATMENT AND PROPHYLAXIS OF EPILEPSY AND FEBRILE SEIZURES

(76) Inventors: Julie A. Kauer, Providence, RI (US); Barry W. Connors, Providence, RI (US); Jennifer A. Kim, Providence, RI (US); Helen E. Gibson, Providence, RI (US)

(21) Appl. No.: 12/879,058

(22) Filed: Sep. 10, 2010

Related U.S. Application Data
(63) Continuation of application No. PCT/US2009/001546, filed on Mar. 11, 2009.
(60) Provisional application No. 61/035,919, filed on Mar. 12, 2008.

(51) Int. Cl. A61K 31/357; A61K 31/55; A61K 31/454; A61K 31/164; A61K 31/165; A61K 31/7088; A61P 25/08; A61P 25/00

(52) U.S. Cl. 514/44 A; 514/213.01; 514/326; 514/450; 514/627

(57) ABSTRACT

Provided are methods for treatment and prophylaxis of convulsive disorders and seizures, such as epilepsy and febrile seizures, by modulating TRPV1 channel activation.

### RELATED APPLICATIONS

This application a continuation of international application PCT/US2009/001546, filed Mar. 11, 2009, which was published under PCT Article 21(2) in English, and claims the benefit under 35 U.S.C. §119(e) of U.S. provisional application 61/035,919, filed Mar. 12, 2008, the entire disclosures of which are incorporated herein by reference.

### GOVERNMENT SUPPORT

This invention was made in part with government support under National Institutes of Health grants DA11289, NS050570 and NS049779. The government has certain rights in this invention.

### FIELD OF THE INVENTION

Treatment and prophylaxis of convulsive disorders and seizures, such as epilepsy and febrile seizures, by modulating TRPV1 channel activation.

### BACKGROUND OF THE INVENTION

The TRPV1 channel, also known as vanilloid receptor VR1, was cloned ten years ago and is a member of a large family of calcium-permeable non-selective cation channels (Caterina et al., 1997; Szallasi and Blumberg, 1999). TRPV1 receptors are gated by heat, low pH, or endogenous ligands termed ‘endovanilloids’ including anandamide, lipoxygenase derivatives of arachidonic acid, and long-chain, linear fatty acid dopamines such as N-arachidonyldopamine (NADA) (Caterina et al., 1997; Tominaga et al., 1998; Zygmunt et al., 1999; Hwang et al., 2000; Smart et al., 2000; Huang et al., 2002; Shin et al., 2002; De Petrocellis and Di Marzo, 2005; Matta et al., 2007). In the peripheral nervous system (PNS), TRPV1 receptors are activated by thermal and chemical stimuli, by capsaicin (8-methyl-N-vanillyl-6-nonenamide; the pungent ingredient of red hot chili peppers), and by the Euphorbia toxin, resiniferatoxin, causing pain, inflammation and hyperalgesia. Bipolar neurons with unmyelinated axons (C-fibres) and somata in dorsal root and trigeminal ganglia, as well as a subset of sensory neurons with thin myelinated axons (AS fibres) are capsaicin-sensitive (Holzer, 1988).

Trauma and genetic disorders can cause seizures and epilepsy, but even the normal brain is capable of having a seizure given appropriate circumstances. During late infancy and early childhood, seizures in an otherwise normal brain can be associated with fevers (>102° F.;>38° C.) independent of CNS infections or other definable causes. Febrile seizures have a prevalence of 3-5%, and usually occur between three months and five years old with peak incidence at 18-24 months (Lowenstein 2005). Patients often have a family history of febrile seizures or epilepsy, and syndromes such as generalized epilepsy with febrile seizures plus (GEFS+) indicate a genetic predisposition (Audenaert et al. 2006, Srinivasan et al. 2005, Waruiru & Appleton 2004). Febrile seizures typically manifest as generalized, tonic-clonic seizures during childhood infections such as middle ear or respiratory infections, orgastroenteritis. Febrile seizures can be categorized as simple or complex. Simple febrile seizures are single, isolated (<15 min), brief, symmetric events. Complex febrile seizures last longer (>15 min), and often have multiple episodes and focal features. About 20-30% of febrile seizures are complex (Stafstrom 2002). One-third of patients experience recurrence, but less than 10% have three or more episodes (Lowenstein 2005, Srinivasan et al. 2005, Waruiru &Appleton 2004). Although clinical outcomes after febrile seizures are generally very good, there are still ongoing investigations of their link to later onset epilepsy. Evidence from animal models of complex febrile seizures and temporal lobe epilepsy patient histories of febrile seizures implicate a relationship between febrile seizures and later onset epilepsy, thereby warranting further investigation. It is clear that febrile seizures are an important clinical problem.

### SUMMARY OF THE INVENTION

As is described below, TRPV1 channel activation is necessary and sufficient to trigger long-term synaptic depression (LTD). Modulation of TRPV1 channel activation provides a way to treat (including prophylaxis of) convulsive disorders and seizures, such as epilepsy and febrile seizures.

According to one aspect of the invention, methods for treatment or prophylaxis of epilepsy are provided. The methods include administering to a subject having epilepsy, suspected of having epilepsy or at risk of developing epilepsy an amount of a TRPV1 antagonist effective to reduce epileptic seizures or prevent the onset of epileptic seizures. In some embodiments the TRPV1 antagonist is capsazepine, SR141716A, or 5′-Iodoresiniferatoxin. In certain embodiments, the TRPV1 antagonist is administered orally, sublingually, buccally, intranasally, intravenously, intramuscularly, intrathecally, intraperitoneally, or subcutaneously.

According to another aspect of the invention, methods for treatment or prophylaxis of epilepsy are provided. The methods include administering to a subject having epilepsy, suspected of having epilepsy or at risk of developing epilepsy an amount of a TRPV1 agonist effective to reduce epileptic seizures or prevent the onset of epileptic seizures. In some embodiments, the TRPV1 agonist is resiniferatoxin, tinyatoxin, anandamide, capsaicin or a caps aicinoid. In certain embodiments, the TRPV1 agonist is administered orally, sublingually, buccally, intranasally, intravenously, intramuscularly, intrathecally, intraperitoneally, or subcutaneously.

According to another aspect of the invention, methods for treatment or prophylaxis of epilepsy are provided. The methods include administering to a subject having epilepsy, suspected of having epilepsy or at risk of developing epilepsy an amount of a molecule that reduces the expression of TRPV1 effective to reduce epileptic seizures or prevent the onset of epileptic seizures. In some embodiments, the molecule that reduces the expression of TRPV1 is molecule that produces RNA interference, preferably a siRNA molecule or a shRNA molecule. In certain embodiments, the molecule that reduces the expression of TRPV1 is administered orally, sublingually, buccally, intranasally, intravenously, intramuscularly, intrathecally, intraperitoneally, or subcutaneously.

According to another aspect of the invention, methods for treatment or prophylaxis of febrile seizures are provided. The methods include administering to a subject having a febrile seizure, suspected of having a febrile seizure or at risk of developing a febrile seizure an amount of a TRPV1 antagonist effective to reduce the febrile seizure or prevent the onset of the febrile seizure. In some embodiments the TRPV1 antagonist is capsazepine, SR141716A, or 5′-Iodoresiniferatoxin. In certain embodiments, the TRPV1 antagonist is administered orally, sublingually, buccally, intranasally, intravenously, intramuscularly, intrathecally, intraperitoneally, or subcutaneously.

According to another aspect of the invention, methods for treatment or prophylaxis of febrile seizures are provided. The methods include administering to a subject having a febrile seizure, suspected of having a febrile seizure or at risk of developing a febrile seizure an amount of a TRPV1 agonist effective to reduce the febrile seizure or prevent the onset of the febrile seizure. In some embodiments, the TRPV1 agonist is resiniferatoxin, tinyatoxin, anandamide, capsaicin or a capsaicinoid. In certain embodiments the TRPV1 agonist is administered orally, sublingually, buccally, intranasally, intravenously, intramuscularly, intrathecally, intraperitoneally, or subcutaneously.

According to another aspect of the invention, methods for treatment or prophylaxis of febrile seizures are provided. The methods include administering to a subject having a febrile seizure, suspected of having a febrile seizure or at risk of developing a febrile seizure an amount of a molecule that reduces the expression of TRPV1 effective to reduce the febrile seizure or prevent the onset of the febrile seizures. In some embodiments, the molecule that reduces the expression of TRPV1 is molecule that produces RNA interference, preferably a siRNA molecule or a shRNA molecule. In certain embodiments, the molecule that reduces the expression of TRPV1 is administered orally, sublingually, buccally, intranasally, intravenously, intramuscularly, intrathecally, intraperitoneally, or subcutaneously.

These and other aspects of the invention are described further below.

### BRIEF DESCRIPTION OF THE FIGURES

FIG. 1. LTD at excitatory synapses on interneurons is NMDAR-independent and is maintained by a decrease in presynaptic glutamate release.
A. A single experiment illustrating interneuron LTD. NMDARs were blocked throughout the experiment using 50 μM D-AP5. At the arrow, HFS was delivered to the afferent pathway. The dotted line in this and all other single examples is an approximation of the mean EPSC response before HFS. Right panel: average of 10 consecutive EPSCs taken just before (black) and 20 minutes after HFS (gray). Calibration: 100 pA, 10 msec.
B. Left panel; Averaged LTD experiments in the presence of 50 μM D-AP5 (n=26). The dotted line in this and all other time course averages represents the mean normalized EPSC value before HFS. Right panel; Averaged LTD experiments in which LTD was not triggered until 40 minutes following break-in to the whole-cell configuration, showing that the ability to induce LTD does not “wash out” over this time period (n=3). This and all other experiments in the paper were carried out in the presence of 50 μM D-AP5. Error bars in this and all figures indicate mean±s.e.m.
C, D, E. Consistent with a presynaptic mechanism, 1/CV² (squared mean EPSC amplitude divided by EPSC variance) decreased, the PPR (EPSC2/EPSC1) increased and the number of synaptic failures increased significantly during interneuron LTD (average synaptic failures pre-HFS: 39.6±3.3%; average synaptic failures post-HFS: 98.5±0.7%; P<0.001, n=6). The paired-pulse ratio and coefficient of variation were calculated for 5 minute epochs before and between 15-20 minutes after HFS (see methods), and control cells with LTD of at least 10% in response to HFS were included in the PPR and 1/CV² analysis. Non-normalized values of 1/CV² (C), PPR (D) and synaptic failures (E) from each interneuron are shown (open circles). The thick black line and filled circles indicate the mean value for all cells. Using non-normalized values, all points are significantly different from pre-LTD values (P<0.05). Inset (D): Example traces of EPSCs taken just before (black) and after HFS-induced LTD (gray) are shown, with the latter scaled so that the first EPSCs are of the same size, illustrating the increased paired pulse ratio during LTD. Calibration: 100 pA, 10 msec. Inset (E): Example traces illustrating consecutive EPSCs evoked using minimal stimulation before and during LTD from one experiment showing EPSCs identified as synaptic failures (gray). Calibration: 25 pA, 25 msec. Stimulus artifacts have been truncated for clarity.
F. NMDAR-mediated EPSCs were evoked while holding the interneuron at +40 mV, in the absence of D-AP5, and including 10 μM 6,7-dinitroquinoxaline-2,3-dione (DNQX) in the bathing solution. At the arrow, HFS was delivered to the afferents with the interneuron in current-clamp mode. Inset: average of 10 EPSCs recorded just before (black) and at 20 minutes after HFS (gray). Calibration for inset: 300 pA, 20 msec.
G. Averaged experiments showing LTD of NMDAR EPSCs recorded by holding the interneuron at +40 mV in the presence of 10 μM DNQX (n=7).

FIG. 2. A group I mGluR antagonist and SR141716A block LTD, but AM251 does not.
A. Averaged data showing that when the group I mGluR antagonist, CPCCOEt (25-50 μM) was bath-applied for at least 10 minutes before HFS (arrow), LTD was blocked in all but one cell (n=9). Inset: average of 10 EPSCs from an example neuron before (black) or 20 minutes after HFS (gray). Calibration for all insets: 100 pA, 10 msec. B. Averaged data showing that SR141716A (2-5 μM) consistently blocked LTD (n=10). Inset: 10 consecutive EPSCs from an example neuron were averaged before (black) or 20 minutes after HFS (gray).
C. The CB1 receptor antagonist, AM251 (2 μM) did not affect LTD (average of 8 experiments). Inset: average of 10 EPSCs taken from an example neuron just before (black) and at 20 minutes after HFS (gray).
D. The CB1 receptor agonist, WIN 55,212-2 (1 μM) was bath-applied and depresses synaptic transmission at excitatory synapses onto interneurons (n=14). Inset: 10 consecutive EPSCs taken from an example neuron were averaged before (black) or 10 minutes after the addition of WIN 55,212-2 (gray).
E. The CB 1 receptor antagonist, AM251 (2 μM), bath applied for at least 10 minutes prior to the addition of WIN 55,212-2 prevents synaptic depression (n=5). Inset: average of 10 EPSCs taken from an example neuron just before (black) and at 10 minutes after the addition of WIN 55,212-2 in the continued presence of AM251 (gray).

FIG. 3. Capsaicin mimics interneuron LTD via TRPV1 receptors and TRPV1 receptor antagonists block LTD induction.
A. A single example illustrating that 12 minutes of bath-applied capsaicin (1 μM) depresses EPSC amplitudes so that no further depression is elicited following HFS (arrow). Right Inset: Top panel; averaged EPSCs taken just before (black) and after 10 minutes in capsaicin (gray). Lower panel; average of 10 EPSCs in capsaicin taken just before (black) and at 20 minutes after HFS (gray). Calibration for all insets: 100 pA, 10 msec.
B. Left panel: Twelve minutes of bath-applied capsaicin (1 μM) depresses EPSC amplitudes (average of 14 experiments). Right panel: After capsaicin (1 μM) caused a stable EPSC depression, HFS was delivered (arrow) but elicited no further depression (average of 10 experiments).
C. Capsaicin (1 μM) application is associated with an increase in synaptic failures using minimal stimulation (P<0.01). Percent failures for 5 experiments are shown for the 10 minute baseline period just before capsaicin application and for the last 5 minutes in capsaicin. The thicker black line and filled circles represent the average of five experiments.
D. SR141716A (2 μM) prevents the synaptic depression by capsaicin (1 μM), as expected if SR141716A is blocking the capsaicin-sensitive receptors (average of 6 experiments). SR141716A was bath applied for at least 10 minutes before the application of capsaicin. Inset: averaged EPSCs from an example neuron in SR141716A before (black) and after 10 minutes in capsaicin (gray).
E. Interneuron LTD was blocked by the TRPV1 receptor antagonist capsazepine (10 μM), bath-applied prior to HFS (arrow) (average of 9 experiments). Inset: average of 10 EPSCs from an example neuron taken just before (black) and at 20 minutes after HFS (gray).
F. Bath-applied 5′-Iodoresiniferatoxin (100 nM), another TRPV1 receptor antagonist, also blocked LTD (average of 7 experiments). Inset: average of 10 EPSCs from an example neuron taken just before (black) and at 20 minutes after HFS (gray).

FIG. 4. Slices from TRPV1^{-/-} mice lack interneuron LTD and capsaicin-induced synaptic depression.
A. In hippocampal slices from TRPV1^{-/-} mice, HFS does not elicit LTD. Left panel, single experiment. Inset: averaged EPSCs before and 15 minutes after HFS. Calibration for all figure insets: 100 pA, 10 msec. Right panel, averaged experiments from TRPV1^{-/-} mice (n=9 animals).
B. In slices from wild-type mice, HFS induces LTD. Left panel, single experiment. Inset: averaged EPSCs before and 15 minutes after HFS. Right panel, averaged experiments from wild-type mice (n=15 animals). Experiments were interleaved with those from TRPV1^{-/-} mice.
C. Capsaicin (1 μM) has no effect on interneuron synapses in slices from TRPV1^{-/-} animals. Left panel, single experiment. Inset: averaged EPSCs before and after 10 minutes in capsaicin. 1 μM capsaicin was added as marked by the bar. Right panel, averaged experiments from slices from TRPV1^{-/-} mice (n=8 animals).
D. In slices from C57BL/6 wild-type mice, capsaicin (1 μM) elicits synaptic depression. Left panel, single experiment. Inset: averaged EPSCs before and after 10 minutes in capsaicin. Right panel, averaged experiments from slices from C57BL/6 wild-type mice (n=6 animals).

FIG. 5. The endogenous TRPV1 receptor agonist 12-(S)-HPETE mimics LTD.
A. The endogenous TRPV1 receptor agonist 12-(S)-HPETE (100 nM) was bath applied for 15 minutes and depressed EPSC amplitudes (average of 8 experiments). Inset: average of 10 EPSCs taken from an example neuron just before (black) and at 10 minutes after 12-(S)-HPETE application (gray). Calibration for this and all insets: 100 pA, 10 msec.
B. Following the bath application of 12-(S)-HPETE for 15 minutes, resulting in a stable EPSC depression, HFS (arrow) failed to induce further LTD (average of 6 experiments). Inset: average of 10 EPSCs in 12-(S)-HPETE taken from an example neuron just before (black) and at 20 minutes after HFS (gray).
C. 12-(S)-HPETE (100 nM) application is associated with an increase in synaptic failures using minimal stimulation (P<0.001). Percent failures for 6 experiments are shown for the 10 minute baseline period just before 12-(S)-HPETE application and for the last 5 minutes in 12-(S)-HPETE. The thicker black line and filled circles represent the average of six experiments.
D. Bath-applied baicalein (500 nM), a 12-lipoxygenase inhibitor, blocked LTD induction (average of 10 experiments). Inset: averaged EPSCs taken from an example neuron before (black) or 20 minutes after HFS (gray) in the presence of baicalein.
E. The TRPV1 receptor antagonist capsazepine (10 μM) prevents the synaptic depression caused by 12-(S)-HPETE (100 nM), as expected if 12-(S)-HPETE acts as a TRPV1 receptor agonist (average of 6 experiments). Inset: averaged EPSCs taken from an example neuron in capsazepine before (black) and after 10 minutes in 12-(S)-HPETE (gray).
F. SR141716A (2 μM) prevents the synaptic depression resulting from the application of 12-(S)-HPETE (100 nM) (average of 5 experiments). Inset: averaged EPSCs taken from an example neuron in SR141716A before (black) and after 10 minutes in 12-(S)-HPETE (gray).
G. 12-(S)-HPETE (100 nM) has no effect on interneuron synapses in slices from TRPV1^{-/-} animals in a single experiment. Inset: averaged EPSCs before and after 10 minutes in 12-(S)-HPETE. 12-(S)-HPETE was added as marked by the bar.
H. 12-(S)-HPETE (100 nM) has no effect on interneuron synapses in slices from TRPV1^{-/-} animals. Averaged experiments from slices from TRPV1^{-/-} mice showing the lack of effect of 1 μM 12-(S)-HPETE (n=6 animals).

FIG. 6. Field potential recordings from synapses on CA1 pyramidal cells are unaffected by concentrations of capsaicin or 12-(S)-HPETE that depress synapses on CA1 interneurons.
A. Field potentials (fEPSPs) recorded at the excitatory synapses between CA3 and CA1 pyramidal cells are not affected by capsaicin (1 μM). Inset: averaged fEPSPs taken from a single experiment before (black) and after 15 minutes in capsaicin (gray). Calibration for the insets: 250 μV, 10 msec.
B. Field potentials (fEPSPs) recorded at the excitatory synapses between CA3 and CA1 pyramidal cells are not affected by 12-(S)-HPETE (100 nM). Inset: average of 10 fEPSPs taken from a single experiment before (black) and after 15 minutes in 12-(S)-HPETE (gray).

FIG. 7. Intracellular blockade of G-protein signaling or the enzyme 12-lipoxygenase, or chelation of intracellular Ca²⁺ reduces the incidence of LTD triggered by HFS.
A. The amount of synaptic depression present 15-20 minutes following HFS is plotted for interneurons in four separate conditions: 1. control intracellular patch pipette solution (control LTD; open circles, n=26), 2. patch pipette solution containing 250 μM GDPβS (filled circles, n=10), 3. patch pipette solution containing 25-40 mM BAPTA (filled circles, n=14), and 4. patch pipette solution containing 140 nM baicalein (filled circles, n=12). Each circular symbol represents the LTD observed in one experiment. Interneurons were held in the whole-cell recording configuration for at least 15 minutes before delivering HFS. The mean for each population is indicated by the horizontal black bar within each set of points. The dotted line in this figure represents the mean normalized EPSC value before HFS. Although the average amount of LTD elicited with each intracellular drug is significantly different from control LTD induced using control intracellular patch pipette solution (* P<0.05, ** P<0.01), in each case there are a few cells that appear to undergo LTD (EPSC amplitudes 15-20 minutes post-HFS with intracellular GDPβS: 88.2±10.6% of control values before HFS; P<0.05 compared to control LTD, n=10; 6 of 10 cells recorded from with intracellular GDPβS had LTD. EPSC amplitudes 15-20 minutes post-HFS with intracellular BAPTA: 90.5±8.5% of control values before HFS; P<0.01 compared to control LTD, n=14, 6 of 14 cells recorded from with intracellular BAPTA had LTD. EPSC amplitudes 15-20 minutes post-HFS with intracellular baicalein: 110.8±15.4% of control values before HFS; P<0.05 compared to control LTD, n=12; 4 of 12 cells recorded from with intracellular baicalein had LTD).

FIG. 8. Functional TRPV1 receptors are found on pyramidal cells and interneurons, but the TRPV1 receptor necessary for LTD is not located on the recorded interneuron.
A. CA1 and CA3 pyramidal cells exhibit a greater peak inward current response to capsaicin (3 μM) when compared to CA1 interneurons. The holding current in three different hippocampal neuron classes was monitored while capsaicin was bath-applied. The peak (mean±s.e.m.) capsaicin response (black bars) was significantly reduced in the presence of the TRPV1 receptor antagonist, capsazepine (10 μM; bars marked ‘+’) in CA1 and CA3 pyramidal cells. Peak capsaicin response in the presence of capsazepine: in CA1 pyramidal cells: 0.1±0.1 pA; P<0.05 compared to that without capsazepine, n=5; in CA1 interneurons: 0.4±0.2 pA; P=0.06 compared to that without capsazepine, n=5; and in CA3 pyramidal cells: 0.1±0.1 pA; P<0.05 compared to that in the absence of capsazepine, n=5). In separate experiments, 1 μM capsaicin elicited a small and variable response in pyramidal cells but essentially no response in interneurons (1 μM capsaicin response in interneurons: 0.7±0.3 pA; P=0.78 compared to pre-drug control values, n=5, data not shown).
B. Intracellular capsazepine does not block LTD. Average of 7 experiments with capsazepine (2 μM) included in the intracellular patch pipette solution. After at least 15 minutes, HFS was delivered (at the arrow). Inset: average of 10 EPSCs taken from an example neuron just before (black) and at 20 minutes after HFS (gray). Calibration for all insets: 100 pA, 10 msec.
C. Intracellular capsazepine does not prevent capsaicin-induced synaptic depression. Average of 6 experiments with capsazepine (2 μM) included in the intracellular patch pipette solution. After at least 15 minutes, capsaicin (1 μM) was bath applied to the slice (bar). Inset: average of 10 EPSCs taken from an example neuron just before (black) and after 10 minutes in capsaicin (gray).
D. Possible scheme to account for the induction of LTD at excitatory synapses onto CA1 interneurons. Glutamate release during synaptic stimulation activates mGluR1/5 receptors, leading to the activation of phospholipase C. Arachidonic acid is converted to 12-(S)-HPETE by a pathway requiring 12-lipoxygenase. 12-(S)-HPETE then activates TRPV1 receptors on presynaptic excitatory nerve terminals. Glutamate release is persistently altered, perhaps by a Ca²⁺-activated signaling cascade.

FIG. 9: Seizure susceptibility in TRPV1^{-/-} vs wild-type.
Top row: Seizure threshold temperature.
Bottom: Seizure onset time.

FIG. 10. Trpv1^{-/-} mice have higher hyperthermic seizure thresholds than wild-type mice.

a, Rectal temperatures immediately prior to heating (baseline) and at the onset of heat-induced seizures are lower in wild-type (left, n=9) than in trpv1^{-/-} mice (right, n=14); p<0.01. Bold line represents the mean.

b, Mean latency (sec) to seizure onset from the beginning of heating is also greater in trpv1^{-/-} mice, p<0.001.

FIG. 11. Trpv1 mice exhibit a lower incidence of heat-induced MUA in area CA3.

a, Sample trace of heat-induced increase in MUA (high-pass filtered at 500 Hz) in CA1 of a wild-type mouse, with magnification of the trace in the inset.

b, Percentage of slices showing increase in MUA during high temperature is greater in slices from wild-type mice (black bars, n=36) compared to trpv1^{-/-} mice (open bars; p<0.05, n=43), or during bath application of capsazepine (grey bars; p<0.05, n=27) recorded from area CA3 stratum pyramidale, while in area CA1 a significant difference was not observed (p>0.1).

FIG. 12. CA1 pyramidal neurons display a heat-activated TRPV1-dependent inward current.

a, Example of simultaneously recorded a temperature (upper trace) on holding current in a CA1 pyramidal neuron (lower trace) while ramping temperature.

b, Bath-applied capsazepine (10 μM) blocked the heat-evoked current.

c, Mean holding current vs. temperature for CA1 pyramidal neurons in the absence (black symbols) or presence (open symbols) of 10 μM bath-applied capsazepine.

d, Average data indicating peak heat-evoked inward current (black bar) is significantly blocked by bath-applied capsazepine (10 μM; open bar, p<0.001).

e, Example trace showing simultaneous measurement of temperature (upper trace) and membrane potential (lower trace) in a CA1 pyramidal neuron.

f, Dependence of membrane potential on temperature in CA1 neurons in the absence (black symbols, 19.7±4.5 mV, n=7) or presence of 2 μM intracellular capsazepine (open symbols, 1.0±0.7 mV, n=6; p<0.01). All recordings are from neurons in wild type mice.

FIG. 13. trpv1 expression is required for heat-activated currents in CA1 and CA3 pyramidal neurons.

a, Heat ramps activate TRPV1 channels in wild-type CA1 pyramidal neurons in vitro (peak current, 114.5±11.0 pA, n=7).

b, Heat ramps activate much smaller currents in CA1 neurons from trpv1^{-/-} mice (peak current, 34.6±8.4 pA, n=5; p<0.001 compared to wild-type).

c, Holding current vs. temperature for CA1 neurons from wild-type (black symbols) and trpv1^{-/-} mice (open symbols).

d, Average peak heat-evoked inward current in CA1 neurons from wild-type mice (black bar), and from trpv1^{-/-} mice in the absence (open bar) or presence (gray bar) of 10 μM ruthenium red (peak current in ruthenium red, 9.3±7.7 pA, n=5, p<0.05).

e, Heat ramps activate inward currents in CA3 pyramidal neurons of wild-type mice (peak current, 106.1±10.1 pA, n=7).

f, Heat-activated currents are much smaller in CA3 neurons from trpv1^{-/-} mice (peak current, 41.2±6.9 pA; p<0.001). g, Holding current vs. temperature for CA3 neurons from wild-type (black symbols) and trpv1^{-/-} mice (open symbols).

h, Average peak heat-evoked inward current in CA3 neurons from wild-type mice (black bar), and from trpv1^{-/-} mice (open bar).

FIG. 14. Once MUA were initiated, the time course and amplitude of MUA did not differ between wild type and trpv1^{-/-} mice.

Time-course of positive trials showing increased MUA in wild-type (black symbols) and trpv1^{-/-} (open symbols) animals at CA1 and CA3 stratum pyramidale.

FIG. 15.

a, The TRPV1 agonist, capsaicin (3 μM, bath-applied) induces inward current in a CA1 neuron (top). Subsequent application of capsaicin plus the TRPV1 receptor antagonist capsazepine (10 μM) does not induce current. Lower panel shows summary data for peak capsaicin-evoked current in the absence (black bar) or presence (open bar) of bath-applied capsazepine.

b, The endogenous TRPV1 activator, 12-(S)-HPETE (100 nM, bath-applied), induces an inward current that is blocked by capsazepine (10 μM) (top). Lower panel shows summary data for peak 12-(S)-HPETE-evoked current in the absence (black bar) or presence of capsazepine (middle, open bar) or from slices from trpv1^{-/-} mice (gray bar).

c, Summary of peak membrane potential changes induced by bath-applied capsaicin (3 μM) in CA1 pyramidal cells in the absence (black bar) or presence (gray bar) of 2 μM intracellular capsazepine; p<0.001.

d, Summary of peak heat-evoked inward currents in CA1 neurons from wild-type controls (black bars) and from trpv1^{-/-} mice (open bars) in response to two consecutive experimental temperature ramps.

e, Temperature-dependence of TRPV1 currents from CA1 (black symbols) and CA3 pyramidal neurons (open symbols). Mean holding current values at each temperature from trpv1^{-/-} mice were subtracted from those from wild type mice.

### DETAILED DESCRIPTION OF THE INVENTION

Transient receptor potential (TRP) channels are a large class of membrane nonselective cation channels. Some of these, including TRPV1, are heat activated; the temperature activation ranges of various TRP channels vary (Dhaka 2006 for review). Their thermal sensitivities can be modulated by different mechanisms, including phosphorylation by protein kinase C (PKC; Vellani et al. 2001). Heat-sensitive TRP channels have been extensively studied in the peripheral nervous system (Patapoutian 2003, Tominaga & Tominaga 2005), but recently many types of TRP channels have been localized within the brain. Toth et al. (2005) observed substantial expression of TRPV1 channels in the hippocampus and neocortex. TRP channels have been implicated in various changes that may increase susceptibility to injury. Recently, Shibasaki et al. (2007) found that TRPV4 is constitutively active at physiologic temperatures and their activation leads to depolarization of the resting membrane potential, when comparing wild-type to knockout mice. Blocking TRP channels has also been shown to reduce damage induced by oxygen-glucose deprivation (Lipski 2006). Activation of TRPV1 enhances paired-pulse depression in hippocampus of the Schaffer collateral pathway (Huang 2002; Al-Hayani 2001). This change is thought to be caused by vanilloid receptor activation leading to suppression of EPSCs but not IPSCs in CA1 (Hajos 2002). Interestingly, TRPV1 channels are also expressed in brain endothelium and may play a role in blood brain barrier permeability (Hu et al 2005). These findings demonstrate that TRP channels in the brain have a significant impact on neural excitability. Heat-sensitive TRP channels could be a substrate for heat-induced hyperexcitability, and febrile seizures.

The TRP family of proteins is currently under intense investigation in health and disease because these ion channels respond to a diverse range of stimuli and because of their widespread distribution in a number of organs and tissues. Currently, TRPV1 receptors are a novel therapeutic target in the PNS, and agonists and antagonists are being tested for the treatment of inflammatory and chronic neuropathic pain (Szallasi and Appendino, 2004; Steenland et al., 2006; Szallasi et al., 2006).

In contrast to the well established function of TRPV1 receptors in the PNS, their role in the central nervous system (CNS) is not well defined. The presence of TRPV1 receptors in the mammalian brain has been demonstrated using in situ hybridization and reverse transcription polymerase chain reaction (RT-PCR) (Sasamura et al., 1998; Mezey et al., 2000), immunochemical staining methods (Sanchez et al., 2001; Toth et al., 2005; Cristino et al., 2006) and [³H]resiniferatoxin autoradiography comparing wild-type and TRPV1 receptor knockout mice (Roberts et al., 2004). These studies indicate the presence of potentially functional TRPV1 receptors in brain regions including the thalamic and hypothalamic nuclei, the locus coeruleus, periaqueductal grey and cerebellum, cortical and limbic structures including the hippocampus, the caudate putamen and the substantia nigra pars compacta. Nonetheless, the functional significance of TRPV1 receptor expression in the brain remains elusive, although there is evidence that TRPV1 receptors in the CNS are involved in pain modulation and may serve as useful drug targets (Cui et al., 2006). TRPV1 receptor mRNA and protein are expressed in hippocampal neurons (Sasamura et al., 1998; Roberts et al., 2004; Toth et al., 2005; Cristino et al., 2006) including those of the human hippocampus (Mezey et al., 2000), and functional effects of these receptors have been shown using electrophysiological methods (Al-Hayani et al., 2001; Huang et al., 2002; Marsch et al., 2007). A recent study using mice lacking TRPV1 receptors suggests their involvement in anxiety-related behavior and two behavioral measures of hippocampal-dependent learning, conditioned and sensitized fear (Marsch et al., 2007). Moreover, hippocampal long-term potentiation (LTP) was attenuated in the CA1 region of brain slices from TRPV1 knockout mice, indicating alterations in synaptic circuit function in this brain region, although the mechanism remains unknown (Marsch et al., 2007).

TRPV1 receptors in the CNS are less likely than those in the PNS to be activated by heat or low pH, and therefore it has been suggested that other endogenous ligands of this ion channel, such as the endovanilloids mentioned above, are likely activators (Huang et al., 2002; Marinelli et al., 2003; Van Der Stelt and Di Marzo, 2004; De Petrocellis and Di Marzo, 2005; Marsch et al., 2007). Anandamide and NADA are also members of the endocannabinoid family, activating CB1 receptors as well (Zygmunt et al., 1999; Huang et al., 2002), and it remains unclear whether or not any of these ligands are responsible for the TRPV1-mediated physiological and pathological effects in and outside of the CNS (Van Der Stelt and Di Marzo, 2004).

Synaptic plasticity in the brain is a fundamental process underlying information storage and adaptation to external stimuli (Malenka and Bear, 2004), and the cellular mechanisms underlying synaptic plasticity are of great interest since manipulation of these mechanisms could be used to modify neural function. Plasticity of synapses onto GABAergic interneurons can modify the output of cortical circuits, since interneurons are essential in the precise control of firing of groups of principle cells as well as in network oscillations (Kullmann and Lamsa, 2007; Mann and Paulsen, 2007). Some years ago it was demonstrated that following high-frequency afferent stimulation, excitatory synapses onto CA1 hippocampal interneurons exhibit long-term depression (LTD) (McMahon and Kauer, 1997). Here we report that TRPV1 channel activation is a novel cellular element required for this form of LTD.

As a result of the understanding this new and surprising effect of TRPV1 on LTD, methods for treatment and/or prophylaxis of convulsive disorders and seizures is provided. In particular, methods for treatment and/or prophylaxis of epilepsy and febrile seizures are provided.

In some embodiments of these methods, TRPV1 antagonists are used to inhibit TRPV1 functioning. In other embodiments, TRPV1 agonists are used to cause receptor desensitization and thus to effect a reduction in TRPV1 function. The use of agonists to cause receptor desensitization in, for example, pain treatment is known; agonists are known to be as effective as antagonists in pain treatment.

TRPV1 nucleic acids and polypeptides, and various uses thereof, are described in U.S. Pat. Nos. 6,335,180 and 7,097,991.

TRPV1 modulators (agonists and antagonists) are disclosed, for example, in United States published applications 2008/0058401, 2008/0051454, 2008/0004253, 2007/0259936, 2007/0225275, 2007/0099954, and 2006/0223837, among others. TRPV1 modulators (agonists and antagonists) are disclosed also in PCT published applications WO 2008/006481, WO 2008/011532, WO 2008/005303, WO 2007/142426, WO 2007/124169, WO 2007/120012, WO 2007/109355, WO 2007/066068, WO 2007/065888, WO 2007/065663, WO 2007/065662, and WO 2007/054480, among others. All of the foregoing documents are incorporated by reference for their teachings of TRPV1 modulators.

Specific TRPV1 antagonists include capsazepine, SR141716A, and 5′-Iodoresiniferatoxin. Specific TRPV1 agonists include resiniferatoxin, tinyatoxin, anandamide, capsaicin and capsaicinoids. Additional TRPV1 modulators are known to those skilled in the art.

In still other embodiments of the methods of treatment or prophylaxis, a reduction of expression of TRPV1 is caused. This may be accomplished by a variety of methods known in the art, such as by RNA interference. RNA interference can be produced by the use of a variety of molecules known in the art, e.g., short interfering RNA molecules (siRNA), short hairpin RNA molecules (shRNA), which produce or are themselves double stranded RNA molecules.

RNA interference (RNAi) is a phenomenon describing double-stranded (ds)RNA-dependent gene specific posttranscriptional silencing. Synthetic duplexes of 21 nucleotide RNAs could mediate gene specific RNAi in mammalian cells, without invoking generic antiviral defense mechanisms (Elbashir et al. Nature 2001, 411:494-498; Caplen et al. Proc Natl Acad Sci 2001, 98:9742-9747). Small-interfering RNAs (siRNAs) and micro RNAs (miRNAs) are well known in the art and DNA-based vectors capable of generating siRNA within cells have been developed, which involve transcription of short hairpin (sh)RNAs that are efficiently processed to form siRNAs within cells (Paddison et al. PNAS 2002, 99:1443-1448; Paddison et al. Genes & Dev 2002, 16:948-958; Sui et al. PNAS 2002, 8:5515-5520; and Brummelkamp et al. Science 2002, 296:550-553), specifically targeting endogenously and exogenously expressed genes.

Accordingly, the present invention provides a polynucleotide comprising an RNAi sequence that acts through an RNAi or miRNA mechanism to attenuate or inhibit expression of TRPV1 gene. In one embodiment, the miRNA or siRNA sequence is between about 19 nucleotides and about 75 nucleotides in length, or preferably, between about 25 base pairs and about 35 base pairs in length. In certain embodiments, the polynucleotide is a hairpin loop or stem-loop that may be processed by RNAse enzymes (e.g., Drosha and Dicer).

An RNAi construct contains a nucleotide sequence that hybridizes under physiologic conditions of the cell to the nucleotide sequence of at least a portion of the mRNA transcript for the TRPV1 gene. The double-stranded RNA need only be sufficiently similar to natural RNA that it has the ability to mediate RNAi. The number of tolerated nucleotide mismatches between the target sequence and the RNAi construct sequence is no more than 1 in 5 basepairs, or 1 in 10 basepairs, or 1 in 20 basepairs, or 1 in 50 basepairs. It is primarily important the that RNAi construct is able to specifically target the TRPV1 gene. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, 95%, 98%, 99% or even 100% sequence identity, between the inhibitory RNA and the portion of the target gene is preferred.

Production of polynucleotides comprising RNAi sequences is well known in the art. For example, polynucleotides comprising RNAi sequences can be produced by chemical synthetic methods or by recombinant nucleic acid techniques. Endogenous RNA polymerase of the treated cell may mediate transcription in vivo, or cloned RNA polymerase can be used for transcription in vitro. Polynucleotides of the invention, including wildtype or antisense polynucleotides, or those that modulate target gene activity by RNAi mechanisms, may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of a nitrogen or sulfur heteroatom. Modifications in RNA structure may be tailored to allow specific genetic inhibition while avoiding a general response to dsRNA. Likewise, bases may be modified to block the activity of adenosine deaminase. Polynucleotides of the invention may be produced enzymatically or by partial/total organic synthesis, any modified ribonucleotide can be introduced by in vitro enzymatic or organic synthesis.

Methods of chemically modifying RNA molecules can be adapted for modifying RNAi constructs (see, for example, Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

In certain embodiments, the subject RNAi constructs are “siRNAs.” These nucleic acids are between about 19-35 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex or translation is inhibited. In a particular embodiment, the 21-23 nucleotides siRNA molecules comprise a 3′ hydroxyl group.

In other embodiments, the subject RNAi constructs are “miRNAs.” microRNAs (miRNAs) are small non-coding RNAs that direct post transcriptional regulation of gene expression through interaction with homologous mRNAs. miRNAs control the expression of genes by binding to complementary sites in target mRNAs from protein coding genes. miRNAs are similar to siRNAs. miRNAs are processed by nucleolytic cleavage from larger double-stranded precursor molecules. These precursor molecules are often hairpin structures of about 70 nucleotides in length, with 25 or more nucleotides that are base-paired in the hairpin. The RNAse III-like enzymes Drosha and Dicer (which may also be used in siRNA processing) cleave the miRNA precursor to produce an miRNA. The processed miRNA is single-stranded and incorporates into a protein complex, termed RISC or miRNP. This RNA-protein complex targets a complementary mRNA. miRNAs inhibit translation or direct cleavage of target mRNAs (Brennecke et al., Genome Biology 4:228 (2003); Kim et al., Mol. Cells 19:1-15 (2005).

In certain embodiments, miRNA and siRNA constructs can be generated by processing of longer double-stranded RNAs, for example, in the presence of the enzymes Dicer or Drosha. Dicer and Drosha are RNAse III-like nucleases that specifically cleave dsRNA. Dicer has a distinctive structure which includes a helicase domain and dual RNAse III motifs. Dicer also contains a region of homology to the RDE1/QDE2/ARGONAUTE family, which have been genetically linked to RNAi in lower eukaryotes. Indeed, activation of, or overexpression of Dicer may be sufficient in many cases to permit RNA interference in otherwise non-receptive cells, such as cultured eukaryotic cells, or mammalian (non-oocytic) cells in culture or in whole organisms. Methods and compositions employing Dicer, as well as other RNAi enzymes, are described in U.S. Pat. App. Publication No. 2004/0086884.

The miRNA and siRNA molecules can be purified using a number of techniques known to those of skill in the art. For example, gel electrophoresis can be used to purify such molecules. Alternatively, non-denaturing methods, such as non-denaturing column chromatography, can be used to purify the siRNA and miRNA molecules. In addition, chromatography (e.g., size exclusion chromatography), glycerol gradient centrifugation, affinity purification with antibody can be used to purify siRNAs and miRNAs.

In certain embodiments, at least one strand of the siRNA sequence of an effector domain has a 3′ overhang from about 1 to about 6 nucleotides in length, or from 2 to 4 nucleotides in length. In other embodiments, the 3′ overhangs are 1-3 nucleotides in length. In certain embodiments, one strand has a 3′ overhang and the other strand is either blunt-ended or also has an overhang. The length of the overhangs may be the same or different for each strand. In order to further enhance the stability of the siRNA sequence, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a 2′ hydroxyl significantly enhances the nuclease resistance of the overhang in tissue culture medium and may be beneficial in vivo.

In certain embodiments, a polynucleotide of the invention that comprises an RNAi sequence or an RNAi precursor is in the form of a hairpin structure (named as hairpin RNA, shRNA). The hairpin RNAs can be synthesized exogenously or can be formed by transcribing from RNA polymerase III promoters in vivo. Examples of making and using such hairpin RNAs for gene silencing in mammalian cells are described in, for example, (Paddison et al., Genes Dev, 2002, 16:948-58; McCaffrey et al., Nature, 2002, 418:38-9; McManus et al., RNA 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. It is known in the art that miRNAs and siRNAs can be produced by processing a hairpin RNA in the cell.

In yet other embodiments, a plasmid is used to deliver the double-stranded RNA, e.g., as a transcriptional product. After the coding sequence is transcribed, the complementary RNA transcripts base-pair to form the double-stranded RNA.

Specific TRPV1 RNAi molecules are described in PCT published application WO 2007/045930.

The methods provided herewith also include administering a second pharmaceutical for treating convulsive disorders or seizures, e.g., epilepsy or febrile seizures.

The term “effective amount” of a composition refers to the amount necessary or sufficient for a composition alone, or together with further doses, to realize a desired biologic effect. The desired response, of course, will depend on the particular condition being treated. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or adverse condition being treated, the size of the subject, or the severity of the disease or adverse condition. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons. One of ordinary skill in the art can empirically determine the effective amount without necessitating undue experimentation.

For any compound described herein the therapeutically effective amount can be initially determined from animal models. A therapeutically effective dose can also be determined from data for compounds which are known to exhibit similar pharmacological activities, such as other TRPV antagonists or TRPV agonists. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

As used herein, the terms “treat,” “treated,” or “treating” when used with respect to an adverse condition, such as a disorder or disease, for example, epilepsy, and febrile seizures may refer to a prophylactic treatment which increases the resistance of a subject to development of the adverse condition, or, in other words, decreases the likelihood that the subject will develop the adverse condition, as well as a treatment after the subject has developed the adverse condition in order to fight the disease, or prevent the adverse condition from becoming worse. Desired outcomes may include a stabilization of the condition, a slowdown in progression of the disease or a full disease-free recovery of the subject. Subjects include mammals including primates, particularly humans, veterinary animals, and companion animals.

The compounds described herein may be administered per se (neat) or in the form of a pharmaceutically acceptable salt. If the formulations of the invention are administered in pharmaceutically acceptable solutions, they may routinely contain pharmaceutically acceptable concentrations of salt, buffering agents, preservatives, compatible carriers, adjuvants, and optionally other therapeutic ingredients. The solutions used preferably are sterile.

When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v). The pharmaceutical compositions of the invention contain an effective amount of HBsAg nanoparticles optionally included in a pharmaceutically-acceptable carrier.

Modes of administering the therapeutic agents of the present invention will vary depending upon the specific agents used and the disease being treated, as would either be known to those skilled in the art or can be established by routine experimentation using methods commonly employed in the art. Dependent upon these factors, the agents may be administered orally or parenterally. Parenteral modes of administration include intravenous, intramuscular, subcutaneous, intradermal, intraperitoneal, intralesional, intrapleural, intrathecal, intra-arterial, and into lymphatic vessels or nodes and to bone or bone marrow. The therapeutic agents of the invention may also be administered topically or transdermally, buccally or sublingually, or by a nasal, pulmonary, vaginal, or anal route.

For oral administration, the pharmaceutical compositions can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

The compounds may be administered by inhalation to pulmonary tract, especially the bronchi and more particularly into the alveoli of the deep lung, using standard inhalation devices. The compounds may be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. An inhalation apparatus may be used to deliver the compounds to a subject. An inhalation apparatus, as used herein, is any device for administering an aerosol, such as dry powdered form of the compounds. This type of equipment is well known in the art and has been described in detail, such as that description found in Remington: The Science and Practice of Pharmacy, 19^{th} Edition, 1995, Mac Publishing Company, Easton, Pa., pages 1676-1692. Many U.S. patents also describe inhalation devices, such as U.S. Pat. No. 6,116,237.

“Powder” as used herein refers to a composition that consists of finely dispersed solid particles. Preferably the compounds are relatively free flowing and capable of being dispersed in an inhalation device and subsequently inhaled by a subject so that the compounds reach the lungs to permit penetration into the alveoli. A “dry powder” refers to a powder composition that has a moisture content such that the particles are readily dispersible in an inhalation device to form an aerosol. The moisture content is generally below about 10% by weight (% w) water, and in some embodiments is below about 5% w and preferably less than about 3% w. The powder may be formulated with polymers or optionally may be formulated with other materials such as liposomes, albumin and/or other carriers.

Aerosol dosage and delivery systems may be selected for a particular therapeutic application by one of skill in the art, such as described, for example in Gonda, I. “Aerosols for delivery of therapeutic and diagnostic agents to the respiratory tract,” in Critical Reviews in Therapeutic Drug Carrier Systems, 6:273-313 (1990), and in Moren, “Aerosol dosage forms and formulations,” in Aerosols in Medicine. Principles, Diagnosis and Therapy, Moren, et al., Eds., Elsevier, Amsterdam, 1985.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

### EXAMPLES

### Example 1

### TRPV1 Channels Mediate Long-Term Depression at Synapses on Hippocampal Interneurons

### Summary

TRPV1 (transient receptor potential vanilloid subfamily member 1) receptors have classically been defined as ligand-gated, non-selective cation channels that act as heat-, proton- and ligand-activated integrators of nociceptive stimuli in sensory neurons, and there has been great interest in TRPV1 as a novel therapeutic target for pain relief. TRPV1 receptors have also been identified in the brain, but their physiological role is poorly understood. Here we report for the first time that TRPV1 channel activation is necessary and sufficient to trigger long-term synaptic depression (LTD). Excitatory synapses onto hippocampal interneurons were depressed either by capsaicin, a potent TRPV1 activator, or by 12-(S)-HPETE, an endogenous eicosanoid released during synaptic stimulation, while neither compound affected excitatory synapses onto CA1 pyramidal cells. TRPV1 receptor antagonists also prevented the induction of interneuron LTD. Furthermore, in brain slices from transgenic mice lacking TRPV1 receptors, LTD was absent and neither capsaicin nor 12-(S)-HPETE elicited synaptic depression. Our results suggest that TRPV1 channel activation represents a novel mechanism capable of selectively modifying synapses onto hippocampal interneurons. Like other forms of synaptic plasticity, TRPV1-mediated LTD may have a role in long-term changes in the physiological and pathological behavior of neural circuits during learning and epileptic activity.

### Results

In rat brain slices, AMPA receptor-mediated excitatory postsynaptic currents (AMPAR EPSCs) were locally stimulated and recorded from hippocampal CA1 interneurons in stratum radiatum. Since NMDA receptor (NMDAR) activation is an essential component of many forms of synaptic plasticity, we first asked whether LTD at these synapses requires NMDARs. In the presence of D-AP5 (50 μM), high-frequency electrical stimulation (HFS) of glutamatergic afferents triggered robust depression of EPSCs onto interneurons, indicating that NMDARs are not necessary for LTD induction (FIG. 1A, B; EPSC amplitudes 15-20 minutes post-HFS: 62.0±5.3% of control values before HFS; P<0.001, n=26). These values are similar to those found previously in the absence of D-AP5 (McMahon and Kauer, 1997), and all subsequent experiments were carried out in the presence of the NMDAR antagonist. Stable LTD could be elicited even after 40 minutes in the whole-cell recording configuration (FIG. 1B; EPSC amplitudes 15-20 minutes post-HFS: 52.0±23.2% of control values before HFS; P<0.05, n=3). Hippocampal interneurons are a diverse group of cells, expressing different neuropeptides and with different axonal innervation patterns (Freund and Buzsaki, 1996; Pana et al., 1998). Nonetheless, synaptic depression followed HFS in the majority of interneurons (26/29 experiments), supporting previous findings that distinct interneuron classes in stratum radiatum can express this form of LTD (McMahon and Kauer, 1997).

The most commonly observed mechanisms underlying synaptic depression are a decrease in presynaptic neurotransmitter release or a decrease in postsynaptic receptor number or responsiveness (Malenka and Bear, 2004). When synaptic plasticity results from a change in neurotransmitter release, this is generally accompanied by an altered coefficient of variation of the EPSCs (CV), and changes in the paired-pulse ratio (PPR) and synaptic failure rate (del Castillo and Katz, 1954; Malinow and Tsien, 1990; Manabe et al., 1992). Consistent with this interpretation, we observed a decrease in 1/CV² and an increase in the PPR and number of synaptic failures during LTD (FIG. 1C, D, E). If LTD at interneuron synapses results from a persistent decrease in presynaptic glutamate release, we would also predict depression of the NMDAR-mediated component as well as the AMPAR-mediated component of the EPSC (isolated in the experiments in FIG. 1A, B). We therefore measured the isolated NMDAR-mediated EPSC at +40 mV and found that HFS delivered to the afferents elicited robust LTD of the NMDAR EPSC (FIG. 1F, G; NMDAR EPSC amplitudes post-HFS: 64.2±11.1% of control values before HFS; P<0.001, n=7). Taken together, these findings indicate that LTD is AMPAR- and NMDAR-independent and results from a persistent decrease in presynaptic glutamate release as monitored by both AMPA and NMDA receptors.

How might high-frequency activation of excitatory afferents trigger LTD at interneuron synapses? Neither NMDARs nor AMPARs are necessary for LTD (FIG. 1), but group I metabotropic glutamate receptors (mGluRs) are expressed on these cells (Ferraguti et al., 2004) and will also be activated by the glutamate released during HFS. We found that LTD was entirely blocked in the presence of the selective mGluR1 antagonist, CPCCOEt (25-50 μM; FIG. 2A; EPSC amplitudes 10-15 minutes post-HFS in CPCCOEt: 111.4±17.0% of control values before HFS; P<0.01 compared to control LTD, n=9). Our findings thus far are reminiscent of other recent examples of LTD in which activation of postsynaptic group I mGluRs produces endocannabinoids (Maejima et al., 2001; Gerdeman et al., 2002; Robbe et al., 2002; Chevaleyre and Castillo, 2003, 2004; Ronesi et al., 2004; Kreitzer and Malenka, 2005; Takahashi and Castillo, 2006). Endocannabinoids can act as retrograde messengers, traveling across the synapse to activate presynaptic CB 1 receptors, thereby reducing presynaptic neurotransmitter release (Llano et al., 1991; Pitler and Alger, 1992; Kreitzer and Regehr, 2001; Ohno-Shosaku et al., 2001; Wilson and Nicoll, 2001). To ask whether endocannabinoids might mediate LTD at interneuron synapses, we tested two CB1 receptor antagonists. SR141716A (rimonabant; 2-5 μM) effectively blocked LTD (FIG. 2B; EPSC amplitudes 15-20 minutes post-HFS: 110.6±19.3% of control values before HFS; P<0.01 compared to control LTD, n=10). However, the selective CB1 receptor antagonist, AM251 (2 μM), did not block LTD in any of the 8 interneurons tested (FIG. 2C; EPSC amplitudes 15-20 minutes post-HFS: 48.6±5.3% of control values before HFS, n=8). To confirm that AM251 indeed blocks CB1 receptors under these experimental conditions, we found in separate experiments that AM251 (2 μM) blocked the synaptic depression of these synapses elicited by the CB1 receptor agonist, WIN 55,212-2 (1 μM) (FIG. 2D, E; EPSC amplitudes after 10-15 minutes in WIN 55,212-2 alone: 66.1±7.9% of pre-drug control values; P<0.001, n=14; EPSC amplitudes after 10-15 minutes in both WIN 55,212-2 and AM251: 101.7±8.9% of pre-WIN 55,212-2 control values; P<0.05 compared to WIN55,212-2 depression in the absence of AM251, n=5). In addition, pre-treatment with WIN 55,212-2 (1 μM) for at least ten minutes did not prevent synaptic depression triggered by high-frequency synaptic stimulation (EPSC amplitudes 15-20 minutes post-HFS: 51.3±7.5% of control values in WIN55,212-2 before HFS; P=0.25 compared to control LTD, n=12; data not shown). The block of LTD by SR141716A but not by AM251 was surprising, indicating that CB1 receptors are not necessary for this form of LTD and instead that SR141716A blocks LTD via a CB1 receptor-independent mechanism.

SR141716A may antagonize not only CB1 receptors but also the TRP channel family member, TRPV1 (De Petrocellis et al., 2001). TRPV1 is found in hippocampal neurons (Hajos and Freund, 2002; Roberts et al., 2004; Toth et al., 2005; Cristino et al., 2006; Marsch et al., 2007) and we therefore first tested whether transient application of a TRPV1 agonist mimics LTD induction. The extremely selective TRPV1 agonist capsaicin (1 μM) significantly depressed excitatory synaptic currents in interneurons (FIG. 3A, B; EPSC amplitudes after 10-15 minutes in capsaicin: 73.3±4.6% of pre-drug control values; P<0.001, n=14). If capsaicin maximally activates signaling mechanisms in common with LTD, HFS following the synaptic depression elicited by capsaicin should not cause any further depression. As predicted, HFS after capsaicin exposure failed to produce further LTD (FIG. 3A, B; EPSC amplitudes 15-20 minutes post-HFS: 112.5±21.9% of control values in capsaicin before HFS; P<0.05 compared to control LTD, n=10). This result suggests that the processes underlying LTD induction are fully activated by treatment with capsaicin, again supporting the requirement for TRPV1 channels in LTD. Like synaptically-induced LTD, the synaptic depression elicited by capsaicin was accompanied by an increase in synaptic failures, supporting our hypothesis that LTD at interneuron synapses results from a persistent decrease in presynaptic glutamate release (FIG. 3C; average synaptic failures pre-capsaicin application: 38.4±3.5%; average synaptic failures in capsaicin: 73.5±7.8%; P<0.01, n=5).

We reasoned that if SR141716A blocks LTD by an antagonist action at TRPV1 receptors on hippocampal neurons, then SR141716A should also prevent capsaicin-induced synaptic depression. After pretreatment with SR141716A (2 μM), capsaicin (1 μM) did not depress the synapses (FIG. 3D; EPSC amplitudes after 10-15 minutes in capsaicin and in the presence of SR141716A: 102.8±9.2% of pre-capsaicin control values; P<0.01 compared to capsaicin depression in the absence of SR141716A, n=6). This finding emphasizes that at this concentration SR141716A cannot be regarded as a selective CB1 receptor antagonist, but instead appears to antagonize capsaicin-sensitive receptors, presumably TRPV1 channels. If, as these data suggest, TRPV1 is necessary for LTD induction at interneuron synapses, then TRPV1 antagonists should interfere with LTD. Both capsazepine (10 μM) and 5′-Iodoresiniferatoxin (100 nM) potently blocked LTD when bath applied prior to HFS (FIG. 3E, F; EPSC amplitudes 15-20 minutes post-HFS in capsazepine: 105.6±8.6% of control values before HFS; P<0.001 compared to control LTD, n=9; in 5′-Iodoresiniferatoxin: 96.2±13.6% of control values before HFS; P<0.01 compared to control LTD, n=7). These data support an essential role for TRPV1 receptors in LTD induction. Once LTD is initiated, TRPV1 channel activity is no longer necessary to maintain synaptic depression since following LTD induction, EPSC amplitudes were not restored to basal values by blocking TRPV1 receptors (10 μM capsazepine was added 10 minutes after HFS; EPSC amplitudes 10-15 minutes after adding capsazepine: 50.1±6.1% of pre-HFS values, n=6; data not shown).

The pharmacological data presented above are all consistent with an essential role for TRPV1 channels in the induction of LTD. To further test this hypothesis, we asked whether LTD could be elicited in transgenic mice lacking TRPV1 receptors (TRPV1^{-/-}) (Caterina et al., 2000). LTD was markedly reduced in slices from TRPV1^{-/-} mice, when compared to LTD in interleaved slices from wild-type control mice (FIG. 4A, B; EPSC amplitudes 15-20 minutes post-HFS in TRPV1^{-/-} mice: 95.8±7.0% of control values before HFS; P<0.001 compared to control LTD in wild-type mice, n=9; in C57BL/6 wild-type mice: 52.1±5.2% of control values before HFS, n=15). While application of capsaicin (1 μM) to slices from wild-type mice elicited synaptic depression, this was not seen in slices from TRPV1^{-/-} mice, confirming the lack of functional TRPV1 receptors (FIG. 4C, D; EPSC amplitudes after 10-15 minutes in capsaicin in TRPV1^{-/-} mice: 100.7±6.6% of pre-drug control values; P<0.01 compared to capsaicin response in wild-type mice, n=8; EPSC amplitudes after 10-15 minutes in capsaicin in C57BL/6 wild-type mice: 50.5±12.1% of pre-drug control values, n=6). These data complement the pharmacological evidence, and strongly suggest that TRPV1 channels or TRPV1-containing heteromultimeric channels are signaling components required for interneuron LTD.

How is LTD initiated by high-frequency synaptic stimulation? Our data are consistent with a model analogous to that of endocannabinoid-mediated LTD (Chevaleyre et al., 2006), in which activation of mGluR1 produces a lipid retrograde messenger capable of activating TRPV1 receptors located on presynaptic pyramidal cell terminals. Activation of group I mGluRs can produce both endocannabinoids and eicosanoid metabolites of arachidonic acid, and these endogenous messengers effectively activate TRPV1 receptors (Zygmunt et al., 1999; Hwang et al., 2000; Shin et al., 2002). The eicosanoid, 12-(S)-HPETE, is known to be liberated during electrical stimulation of hippocampal slices (Feinmark et al., 2003), and thus we asked whether or not this lipid messenger can mimic LTD at interneuron synapses. Application of 12-(S)-HPETE (100 nM) depressed excitatory synapses on interneurons (FIG. 5A; EPSC amplitudes after 10-15 minutes in 12-(S)-HPETE: 40.6±11.7% of pre-drug control values; P<0.01, n=8), and subsequent HFS did not produce further LTD (FIG. 5B; EPSC amplitudes 15-20 minutes post-HFS: 98.7±15.3% of control values in 12-(S)-HPETE before HFS; P<0.05 compared to control LTD, n=6). In addition, the synaptic depression as a result of 12-(S)-HPETE application, like that caused by HFS or capsaicin, was associated with an increase in synaptic failures (FIG. 5C; average synaptic failures pre-12-(S)-HPETE application: 35.2±1.9%; average synaptic failures in 12-(S)-HPETE: 89.7±3.0%; P<0.001, n=6). 12-(S)-HPETE synthesis from arachidonic acid requires 12-lipoxygenase. To determine whether or not endogenously released 12-(S)-HPETE is responsible for triggering LTD following synaptic stimulation, we attempted to induce LTD using HFS in the presence of baicalein (500 nM), an inhibitor of 12-lipoxygenase. Synaptically-induced LTD was blocked in the presence of baicalein, and in fact in four of ten cells we observed potentiation (greater than 125% of control 20 minutes following the HFS)(FIG. 5D; EPSC amplitudes 15-20 minutes post-HFS: 129.6±20.3% of control values before HFS; P<0.01 compared to control LTD, n=10). Moreover, the depression caused by 12-(S)-HPETE was prevented by either capsazepine (10 μM) or SR141716A (2 μM) (FIG. 5E, F; EPSC amplitudes after 10-15 minutes in 12-(S)-HPETE and in the presence of capsazepine: 103.0±8.9% of pre-12-(S)-HPETE control values; P<0.01 compared to 12-(S)-HPETE depression in the absence of capsazepine, n=6; in 12-(S)-HPETE and in the presence of SR141716A: 106.9±5.5% of pre-12-(S)-HPETE control values; P<0.01 compared to 12-(S)-HPETE depression in the absence of SR141716A, n=5). These observations demonstrate a similar pharmacological profile for 12-(S)-HPETE and synaptically-triggered LTD. Finally, we found that in slices from TRPV1^{-/-} mice, 12-(S)-HPETE did not depress synaptic transmission at excitatory synapses on interneurons (FIG. 5G, H; EPSC amplitudes after 10-15 minutes in 12-(S)-HPETE in TRPV1^{-/-} mice: 109.0±6.8% of pre-drug control values; P=0.33, n=6). To rule out any possible involvement of the retrograde messenger, nitric oxide (NO), we tested whether or not LTD was affected when nitric oxide synthase (NOS) was inhibited. When 200 μM L-NAME was bath applied 10 minutes prior to HFS, LTD appeared entirely normal, suggesting that NO does not have a role in this form of synaptic plasticity (EPSC amplitudes 15-20 minutes post-HFS: 46.3±10.4% of control values before HFS, n=5; data not shown). Together, our data strongly suggest that 12-(S)-HPETE acts at TRPV1 receptors to depress synaptic transmission at excitatory synapses onto interneurons, and that 12-(S)-HPETE liberated during HFS is essential for triggering LTD.

Interneurons in stratum radiatum of hippocampal area CA1 receive their major excitatory synaptic inputs from CA3 pyramidal cells but can also receive recurrent collaterals from CA1 pyramidal cells (Freund and Buzsaki, 1996). We next tested whether or not field excitatory postsynaptic potentials (fEPSPs) from synapses between CA3 pyramidal cells and CA1 pyramidal cells also exhibit TRPV1-mediated synaptic depression. Surprisingly, 1 μM capsaicin, a concentration that significantly depressed excitatory synapses on interneurons (FIG. 3A, B), did not depress synapses on CA1 pyramidal cells (FIG. 6A; fEPSP slopes after 10-15 minutes in capsaicin: 102.8±5.4% of pre-drug control values; P=0.58, n=5). Although 10 μM capsaicin depressed synaptic transmission at the CA3-CA1 synapse, as previously reported (Hajos and Freund, 2002), we found that this was often associated with a depression in the presynaptic fiber volley component of the field potential, suggesting a possible confounding effect on presynaptic excitability. Furthermore, we found that 100 nM 12-(S)-HPETE, a concentration that significantly depressed excitatory synapses on area CA1 interneurons (FIG. 5A), did not depress synapses between CA3 and CA1 pyramidal cells (FIG. 6B; fEPSP slopes after 10-15 minutes in 12-(S)-HPETE: 99.0±3.5% of pre-drug control values; P=0.83, n=5). Our data strongly suggest that TRPV1 channel activation does not depress glutamate release at these CA3 excitatory synapses onto CA1 hippocampal pyramidal cells, but potently inhibits excitatory synapses on interneurons in area CA1 stratum radiatum.

We next investigated the involvement of the recorded interneuron in the generation of LTD. We found that intracellular perfusion of recorded interneurons with either GDPβS (250 μM), to block G-protein signaling, or BAPTA, (25-40 mM) to chelate postsynaptic Ca²⁺, reduced interneuron LTD (FIG. 7), a result that can be explained if lipid retrograde messengers required for LTD are largely produced by the interneuron. Furthermore, delivery of the 12-lipoxygenase inhibitor, baicalein (140 nM) into the postsynaptic neuron via the patch pipette also markedly attenuated LTD (FIG. 7). Together, these data indicate that the 12-(S)-HPETE necessary for LTD induction is produced in the recorded interneuron, and that G-protein signaling and postsynaptic Ca²⁺ play an important role.

Where are the TRPV1 receptors located that must be activated during LTD? Capsaicin was bath applied to determine whether we could detect TRPV1-mediated inward currents in different types of hippocampal neurons. Following bath application of 3 μM capsaicin, inward currents were elicited in both CA3 and CA1 pyramidal cells (FIG. 8A, black bars; peak capsaicin response in CA1 pyramidal cells: 160.1±55.3 pA, n=7; in CA3 pyramidal cells: 180.1±48.0 pA, n=5). In contrast, CA1 stratum radiatum interneurons consistently exhibited little or no response to 3 μM capsaicin (FIG. 8A, black bar; peak capsaicin response in interneurons: 41.6±16.2 pA; P<0.05 compared to CA1 and CA3 pyramidal cell responses, n=5). In interleaved control experiments, 10 μM capsazepine blocked the effects of 3 μM capsaicin application, indicating that the capsaicin-induced inward currents were caused by TRPV1 receptor activation (FIG. 8A). These results demonstrate the presence of functional TRPV1 receptors on pyramidal cell bodies as well as on some interneurons. The TRPV1 responses on interneurons were variable at best, suggesting that TRPV1 receptors on interneurons themselves do not play a significant role in LTD. To examine this directly, we delivered the TRPV1 receptor antagonist capsazepine (2 μM) into the recorded interneuron where it can inhibit the channel from the inside (Jordt and Julius, 2002). We found intracellular capsazepine to be ineffective at blocking either synaptically induced LTD or capsaicin-triggered depression (FIG. 8B, C; EPSC amplitudes 15-20 minutes post-HFS with intracellular capsazepine: 47.5±10.3% of control values before HFS; P=0.20 compared to control LTD observed using control intracellular patch pipette solution, n=7; EPSC amplitudes after 10-15 minutes in 1 μM capsaicin and in the presence of intracellular capsazepine: 46.8±10.3% of pre-capsaicin control values; P<0.05 compared to capsaicin depression in the absence of intracellular capsazepine, n=6). These data indicate that TRPV1 receptors on interneurons are not necessary for LTD, and are instead consistent with a model in which the TRPV1 receptors responsible for interneuron LTD may be located on the nerve terminals of pyramidal cells (FIG. 8D).

### Discussion

A rapidly growing body of evidence suggests a functional role for the TRPV channel family in brain function (Marinelli et al., 2003; Lipski et al., 2006; Marinelli et al., 2007; Marsch et al., 2007; Shibasaki et al., 2007). In this study we show for the first time that TRPV1 receptors are necessary and sufficient for a novel form of long-term depression at excitatory synapses. The broad distribution of TRPV1 receptors in the brain suggests that these receptors could play a similar role in synaptic plasticity throughout the CNS. TRPV1 receptors may even contribute to some examples of previously reported endocannabinoid-mediated LTD, since anandamide can activate TRPV1 in addition to CB1 receptors.

We also report that in the hippocampus at least, SR141716A appears to be insufficiently selective to distinguish CB1 from TRPV1 receptors. In our study, SR141716A blocked LTD, in addition to responses to capsaicin and to 12-(S)-HPETE, whereas the very similar CB1 receptor antagonist, AM251, was ineffective. SR141716A has been shown to attenuate responses to capsaicin in other systems as well, particularly at concentrations above 1 (Zygmunt et al., 1999; De Petrocellis et al., 2001). A pharmacological profile similar to what we have observed was reported for the vasorelaxation of small mesenteric blood vessels that was mediated by an endothelial receptor in response to NADA, also blocked by SR141716A but not AM251 (O'Sullivan et al., 2004). Our findings may also relate to previous reports of a vanilloid receptor-like response at hippocampal excitatory synapses (Al-Hayani et al., 2001; Hajos and Freund, 2002). SR141716A (also known as rimonabant or Acomplia) is in wide clinical use outside the United States as an anti-obesity aid (Tucci et al., 2006; Padwal and Majumdar, 2007). A large percentage of patients stop taking this drug as a result of psychiatric side-effects, and our findings suggest the possibility that some of the central effects of rimonabant result from the antagonism of TRPV1 receptors as well as CB1 receptors (Pegorini et al., 2006).

TRPV1 receptors are expressed in hippocampal neurons (Mezey et al., 2000; Szabo et al., 2002; Toth et al., 2005; Cristino et al., 2006) and may be activated in several different ways, including by lipoxygenase derivatives that can be released as a result of group 1 mGluR activation, as we have shown here (Hwang et al., 2000; Sohn et al., 2007). 12-(S)-HPETE is known to be released during field stimulation of hippocampal slices (Feinmark et al., 2003), and our data indicate that 12-(S)-HPETE production is necessary and sufficient for LTD at excitatory interneuron synapses. Our previous study showed that LTD was triggered simultaneously at both activated and non-activated synapses on interneurons, indicating that the LTD is not synapse-specific or activity-dependent (McMahon and Kauer, 1997). The heterosynaptic nature of interneuron LTD may be accounted for by the local spread of 12-(S)-HPETE from interneurons activated during HFS. The most likely source of this eicosanoid is the recorded interneuron itself, based on our data using internally-perfused drugs; when applied intracellularly to the interneuron the Ca²⁺ chelator, BAPTA, the G-protein inhibitor, GDPβS, and the 12-lipoxygenase inhibitor, baicalein, all reduced the number of interneurons exhibiting LTD, suggesting that a Ca²⁺-sensitive process, a GPCR-mediated process and 12-lipoxygenase generation within the interneuron are necessary for LTD. If pyramidal cells, whose processes surround stratum radiatum interneurons, were a significant source of 12-(S)-HPETE following HFS, drugs delivered intracellularly to the recorded interneuron should not block LTD. Instead, in most experiments the intracellularly delivered drugs blocked LTD (FIG. 7). However in some interneurons even with BAPTA, GDPβS or baicalein present, LTD of normal magnitude was induced, suggesting that the necessary signaling molecules can also arise elsewhere; we favor the idea that in some cases neighboring interneurons may release sufficient 12-(S)-HPETE to depress synapses, even when postsynaptic processes are blocked in the recorded cell. It is alternatively possible that perhaps the heterogeneity of hippocampal interneurons could also account for these data (Freund and Buzsaki, 1996; Parra et al., 1998).

The simplest model to account for our results is that synaptic stimulation releases glutamate that activates group 1 mGluRs producing 12-(S)-HPETE, which may act as a retrograde messenger (Feinmark et al., 2003). 12-(S)-HPETE in turn may open TRPV1 channels on the presynaptic glutamatergic terminals of CA1 and/or CA3 pyramidal cells that synapse onto interneurons (FIG. 8D). How might activation of a Ca²⁺-permeable ion channel lead to persistent synaptic depression? Calcium entry through TRPV1 channels on glutamatergic terminals could initiate a signaling cascade responsible for the persistent downregulation of glutamate release observed during LTD. In dorsal root ganglion neurons, TRPV1 channel opening triggers calcineurin activation, which then rapidly depresses multiple voltage-gated calcium channels (Wu et al., 2005, 2006). Moreover, presynaptic NMDARs are required for spike-timing dependent LTD in neocortical neurons (Sjostrom et al., 2003) and Ca²⁺ arising from presynaptic activity is required for LTD at striatal synapses (Singla et al., 2007), suggesting that presynaptic Ca²⁺ signals are required to initiate these forms of LTD as well. However, in both of these examples, co-active CB1 receptors are also required for LTD, whereas CB1 receptors are not required for LTD at excitatory synapses onto hippocampal interneurons, since AM251 was ineffective in blocking this form of LTD. The model we present is the simplest to account for all of our data; however, while we report here that functional TRPV1 receptors are present on CA3 and CA1 pyramidal cell bodies, TRPV1 receptors are also expressed in glial cell populations (Doly et al., 2004; Kim et al., 2006), so it remains possible that an alternative, more complex signaling pathway is involved.

TRPV1 was first identified as a heat-sensitive ion channel in peripheral sensory neurons (Caterina et al., 1997). The temperature threshold of 43° C. for TRPV1 channels (Caterina et al., 1997) is normally outside the brain's physiological range, but the sensitivity of the channel to heat and other activating stimuli can be modulated by endogenous lipids and by the phosphorylation state of the channel (Vellani et al., 2001; Benham et al., 2003). It is therefore conceivable that during fever TRPV1 channels in the hippocampus may be activated, producing LTD at interneuron synapses. Depression of these synapses is expected to increase the excitability of innervated pyramidal cells. In this regard, it is intriguing that the in vivo treatment of animals with SR141716A after the induction of febrile seizures reduced hyperexcitability in hippocampal area CA1 and prevented the emergence of long-term limbic hyperexcitability (Chen et al., 2007). Our data suggest that the blockade of TRPV1 receptors could contribute to the anticonvulsant effect of SR141716A. The selective depression of excitatory synapses on interneurons but not on CA1 pyramidal cells that we report suggests that TRPV1 receptors are differentially distributed on hippocampal excitatory afferents and offers the potential to target hippocampal inhibitory circuits selectively through TRPV1 receptors.

Recently there has been great interest in therapeutic agents targeting TRPV1 receptors for several disorders, most notably inflammatory and neuropathic pain (Szallasi and Appendino, 2004; Steenland et al., 2006; Szallasi et al., 2006). Although drugs binding to peripheral TRPV1 receptors exert analgesic effects on their own, there is also evidence that TRPV1 receptors in the CNS are involved in pain modulation and may serve as useful drug targets (Cui et al., 2006). Our results as well as others (Marsch et al., 2007) indicate that drugs that bind to CNS TRPV1 receptors are likely to influence more than just pain-related functions. The human hippocampus expresses relatively high levels of TRPV1 mRNA (Mezey et al., 2000), suggesting that effects such as those reported here in rodent brain may occur in humans as well. Further work will help to ascertain whether hippocampal TRPV1 receptors could provide novel drug targets for neurological disorders.

### TRPV1 In Vivo Experiments:

Experiments were conducted comparing the seizure susceptibility in TRPV1 knockout and wild-type littermate mice. Body weight, gender, rectal temperature prior to experimentation (baseline temperature), time to seizure onset, rectal temperature at seizure onset and general locomotive behavior before, during and after seizure onset were recorded. The experiments were carried out blind to each animal's genotype until after data were analyzed. The Baram model (described in the Methods section) of warm-air induced febrile seizures was used. A mouse was placed in a 3L beaker and warm-air from a 1600 W hairdryer was used to increase body temperature of the mouse. All behaviors were noted, including paw-biting, clonus, etc. The behavioral correlate to generalized seizure activity electrographically is when the mouse assumes a belly-flat prone position for 10 seconds. Seizure susceptibility was tested in 14 knockout and 9 wildtype mice. Rectal temperature measures in wild-type and knockout mice are summarized (FIG. 9A; gray=individual animals; red=mean of knockouts; blue=mean of wild-types; error bars=standard error). Seizure onset time data was also compared (FIG. 9B). Unpaired, two-tailed t-tests reveal that even with the small number of animals tested, there is a statistically significant difference in both threshold temperature and time of seizure onset of knockout to wild-type mice. Seizure threshold temperature measures were significantly higher in knockout mice compared to their wild-type littermates (p=0.0023). Baseline rectal temperature measures were not different between the two groups, consistent with previous studies about body temperature regulation in TRPV1 knockout mice (Iida et al. 2005). Time to seizure onset in knockout mice was greatly prolonged compared to that of wild-type mice (p=0.0009). These results suggest TRPV1 receptors are active at febrile temperatures and impact seizure susceptibility by increasing neuronal depolarization and excitability when present.

### Experimental Procedures

### Preparation of Brain Slices

The basic methods have been detailed previously (McMahon and Kauer, 1997). Sprague-Dawley rats (15-22 days old) were used in the majority of experiments. In addition, we used TRPV1^{-/-} mice (Caterina et al., 2000) and wild-type C57BL/6 mice aged between 15 and 21 days (Jackson Laboratory). The TRPV1^{-/-} mice we used have been backcrossed at least 10 times onto a C57BL/6 background and were obtained from homozygous breeding pairs. Control mice were therefore not littermates but were age-matched, wild-type C57BL/6 animals received from the same supplier in the same shipment. All animal protocols were approved by the Brown University Institutional Animal Care and Use Committee. For mouse experiments, only one brain slice per mouse was used for each experiment, so that reported ‘n’ numbers represent the number of animals. Animals were anaesthetized using halothane or isoflurane and quickly decapitated. The brain was rapidly removed and 300 μm thick coronal slices prepared and stored for at least one hour submerged on a net in artificial cerebrospinal fluid (ACSF) containing in mM: 119 NaCl, 26 NaHCO₃, 2.5 KCl, 1.0 NaH₂PO₄, 2.5 CaCl₂, 1.3 MgSO₄ and 11 dextrose, saturated with 95% O₂/5% CO₂ (pH 7.4). Slices were then transferred to a submerged recording chamber and bathed in oxygenated ACSF (28-32° C.) containing elevated divalent cations to reduce epileptiform activity (4 mM CaCl₂ and 4 mM MgCl₂, replacing MgSO₄). A surgical cut was made between the CA3 and CA1 regions. The storage of slices submerged on a net rather than in an interface chamber on filter paper may be important in maintaining slice health and improving the likelihood of observing LTD.

Electrophysiological Recordings from Interneurons

Slices were continuously perfused with ACSF warmed to 28-32° C. at a flow rate of 1-2 ml/min. Picrotoxin (100 μM) and D-AP5 (50 μM) were added to block GABA_{A} receptor- and NMDAR-mediated synaptic transmission. Whole-cell patch clamp recordings were made from interneurons identified visually in the CA1 stratum radiatum of the hippocampus. No specific cell morphology was targeted, although we did not record from cells with the “giant cell” morphology as these have been reported to be glutamatergic interneurons (Gulyas et al., 1998). Patch pipettes were filled with internal recording solution containing in mM: 117 cesium gluconate, 2.8 NaCl, 5 MgCl₂, 20 HEPES, 2 ATP-Na⁺, 0.3 GTP-Na⁺ and 0.6 EGTA. In some experiments 2 μM capsazepine, 140 nM baicalein, or 250 μM GDPβS were also included in the intracellular patch pipette solution. In experiments with BAPTA-containing patch electrodes, EGTA was omitted from the intracellular solution and 25 or 40 mM BAPTA replaced a corresponding amount of cesium gluconate. EPSCs were stimulated at 0.1 Hz (100 μsec) using a bipolar stainless steel stimulating electrode placed in stratum radiatum at least 200 μm from the recorded cell. CA1 interneurons were voltage clamped at −65 mV (not corrected for the liquid junction potential, of ˜10 mV), and EPSCs were evoked by paired pulses with an interval of 50 msec (stimulus intensity typically 50-400 μA). In early experiments, we measured rectification ratios of EPSCs evoked at +40 mV/−60 mV in the presence of 50 μM D-AP5, measured at the time of peak inward synaptic current seen at −70 mV (Lei and McBain, 2004). Rectification ratios did not correlate with the incidence of LTD: interneurons with no LTD, 0.63±0.19, n=3, range 0.25-0.86; interneurons with transient LTD, 0.47±0.05, n=4, range 0.42-0.52; interneurons with persistent LTD, 0.58±0.11, n=9, range 0.11-1.28.

High-frequency stimulation was used to induce LTD (HFS; two 1 sec trains at 100 Hz, inter-train interval 20 sec, at 1.5 times test current intensity) with the neuron held in current-clamp mode, so that the HFS trains were delivered with the membrane potential free to vary. Receptor antagonists were added directly to the ACSF at known concentrations for at least 10 minutes prior to HFS. Control experiments were interleaved with those experiments using receptor antagonists or involving slices from TRPVl^{-/-} mice. The cell input resistance and series resistance were monitored throughout each experiment; cells were discarded if these values changed by more than 10% during the experiment. EPSCs were amplified using an AxoClamp 2B amplifier (Axon instruments) and Brownlee Precision Model 410 post-amplifier (AutoMate Scientific), low-pass filtered at 3 kHz and digitally sampled to a PC at 30 kHz using an analogue to digital interface (National Instruments).

### Field EPSP Recordings

Extracellular field potential recordings were made from synapses between CA3 and CA1 pyramidal cells in hippocampal slices prepared from rats as previously described (McMahon and Kauer, 1997). Briefly, 400 μm thick coronal slices were cut using a vibratome and individual slices were stored for at least one hour submerged on a net in ACSF. Slices were then transferred to a submersion chamber and held between two nylon nets. The chamber was constantly perfused with high divalent ACSF including 100 μM picrotoxin, oxygenated and warmed to 29-31° C. at a flow rate of ˜2-3 ml/min. A bipolar stainless steel stimulating electrode placed in stratum radiatum was used to stimulate CA1 field potentials, while a recording electrode filled with 2M NaCl was positioned about 500 μm from the stimulating electrode in stratum radiatum. Stimuli (intensity typically 50-200 μA, 100 μsec duration) were delivered at 0.1 Hz and the current intensity was adjusted to elicit a fEPSP of 0.5 mV at the start of each experiment. fEPSPs were amplified using an AxoPatch 1D amplifier (Axon instruments) and Brownlee Precision Model 410 post-amplifier (AutoMate Scientific), low-pass filtered at 1-2 kHz and digitally sampled to a PC at 10-20 kHz using an analogue to digital interface (National Instruments). Capsaicin (1 μM) or 12-(S)-HPETE (100 nM) were added directly to the ACSF bathing solution after at least a 15 minute baseline period of consistent fEPSPs.

### Analysis

The maximal initial slope of fEPSPs was calculated using a LabVIEW-based program (National Instruments). The peak amplitude of each EPSC was measured by comparing a 10 msec time period immediately prior to the stimulus with the peak of the EPSC using this program as well. Occasionally polysynaptic responses were evoked, and in these cases, only the initial monosynaptic event was measured. To positively identify LTD, EPSCs measured every 10 seconds were averaged in 1 minute intervals. EPSC amplitude values were normalized to control pre-HFS EPSC amplitude values (baseline period of at least 5 minutes prior to HFS) and subjected to analysis of variance (ANOVA) repeated measures analysis with a post-hoc Dunnett's test (GraphPad Prism, Version 4). A significant decrease (P<0.05) in EPSC amplitude in 5 minute periods following HFS that persisted more than 10 minutes post-HFS, indicated that LTD had been induced. EPSC amplitude values 15 to 20 minutes post-HFS were compared between control LTD experiments and those carried out either in transgenic TRPV1^{-/-} mice, or in the presence of drug using a t-test (unpaired, two-tailed, with Welch's correction if the variances between the groups were unequal). To calculate the effects of capsaicin, 12-(S)-HPETE or WIN 55,212-2 application on basal excitatory glutamatergic transmission, normalized EPSC amplitudes or fEPSP slopes were averaged in the final 5 minutes of drug application and compared with EPSCs/EPSPs 5 minutes prior to drug application. In addition, to measure capsaicin's effects on holding current, the peak change in holding current was measured during bath application of 3 μM capsaicin. The n-values reported refer to the number of slices. All combined data are expressed as mean±the standard error of the mean (s.e.m.). All results reported in this study were significant to at least P<0.05.

Paired-pulse ratios (PPR; EPSC2/EPSC1) and coefficient of variation (1/CV²) were calculated within 5 minute epochs of 30 EPSCs each, starting 5 minutes immediately before HFS or drug addition. The PPR was calculated by dividing the mean of all 30 EPSC2 amplitudes by the mean of all 30 corresponding EPSC1 amplitudes within each epoch. 1/CV² was determined by dividing the squared mean amplitude of 30 EPSCs within 5 minute epochs by the variance of these EPSC amplitudes. Experiments in which the EPSC was depressed by more than 10% in response to HFS were included in the PPR and 1/CV² analysis. Given that in some of the experiments the synaptic depression following HFS returned to baseline values after 15 to 20 minutes, we are most confident of the PPR and 1/CV² data over the 20 minute time period immediately following HFS. For statistical analysis of significance of the changes in non-normalized values of 1/CV² and PPR, we used distribution-free, non-parametric inferential statistics (Wilcoxon Matched-Pairs Signed-Ranks Test) to assess these values obtained from the same cell before and after HFS with a significance level of P<0.05. Non-parametric statistics were used since the response values did not meet assumptions of normality and homogeneity of variance.

For synaptic failure analysis, EPSCs were evoked using minimal stimulation intensities that resulted in at least 20% failures of synaptic transmission. The number of failures for each experiment was determined by eye for the baseline period of at least 10 minutes; the largest amplitude value associated with a failure was then defined as the threshold value for individual failures in that experiment. This analysis necessarily groups both failures of transmitter release and transmission failures. Failures reported in the figures were assessed as the percentage of failures occurring during a 10 minute control baseline period, for the 15-20 minute time period post-HFS (FIG. 1E) or for the 10-15 minute time period following the application of capsaicin or 12-(S)-HPETE (FIGS. 3C and 5C).

### Materials

SR141716A was generously provided by NIDA. 12-(S)-HPETE [12-(S)-Hydroperoxyeicosa-5Z, 8Z, 10E, 14Z-tetraenoic acid] was purchased from Biomol International and BAPTA [1,2-bis(o-aminophenoxy)ethane-N,N,N′,N′-tetraacetic acid] was purchased from Calbiochem. AM251, baicalein, capsaicin, capsazepine, CPCCOEt [7-(hydroxyimino)cyclopropa[b]chromen-1a-carboxylate ethyl ester], D-AP5 [D(−)-2-amino-5-phosphonovaleric acid], 5∝-Iodoresiniferatoxin, L-NAME and WIN 55,212-2 mesylate were obtained from Tocris Bioscience. All other chemicals were purchased from Sigma-Aldrich. AM251, baicalein, capsaicin, capsazepine, CPCCOEt, 5′-Iodoresiniferatoxin, SR141716A and WIN 55,212-2 mesylate were dissolved in DMSO and then diluted at least 1:1000 to the final concentration in ACSF, or for baicalein and capsazepine, at least 1:5000 to the final concentration in the intracellular patch pipette solution. Control experiments showed that 0.1% DMSO did not block LTD (EPSC amplitudes post-HFS: 67.7±17.8% of baseline values, n=3; not significantly different from control LTD).

### General Methods

### Mice:

TRPV1 knockout mice and wild-type mouse littermates are compared. TRPV1 homozygous knockout mouse breeders are commercially available and can be obtained from Jackson Laboratories. Genotypes of pups are determined by standard methods of tail cutting, extraction and PCR of their DNA. Mouse background is extremely important to consider when conducting any experiment. Dube et al. (2005) found that mice of different backgrounds can have significantly different susceptibility to febrile seizures.

### Age-Dependence:

Previous studies have demonstrated an age dependence of high temperature-induced seizures in rat and mouse pups (Holtzman et al 1981; Tancredi et al. 1992; Dube & Baram 2005). Heat-lamp induced febrile seizures are ideal at P7 (Holtzman et al. 1981). Warm-air induced seizures can be observed through P12 (Baram et al. 1997). In vitro, the best spontaneous and evoked epileptiform activity was elicited at age P13-20 (Tancredi et al. 1992). In a more recent study by Dube et al. 2005, this group found that mice at P14-15 had the most robust behavioral and electrographic seizure activity.

### Preparation of Brain Slices:

Coronal brain slices will be prepared from the mice described above. Methods have been described in detail (e.g., Beierlein et al. 2000, 2003; Deans et al., 2001; Gibson et al 1999; Cruikshank et al., 2007). Briefly, mice will be deeply anesthetized with thiopental (50 mg/kg) and decapitated. The brain will quickly be removed and placed into ice cold artificial cerebrospinal fluid (ACSF: 126 mM NaCl, 3 mM KCl, 1.25 mM NaH2PO4, 26 mM NaHCO3, 10 mM dextrose, 2 mM MgSO4, and 2 mM CaCl₂). 350 μm-thick coronal slices will be made using a vibratome at ˜0-4° C. Slices will then be put in a submersion holding chamber containing aerated ACSF (bubbled with 95% O2 and 5% CO2) at 32° C. for 30-45 min. The slices will then be maintained at room temperature in a holding chamber until transferred to the recording chamber.

### Extracellular Field Potential Recordings:

Recordings are made in the hippocampus and adjacent parahippocampal regions using a gas-liquid interface chamber. Slices are placed on lens paper, continuously superfused with oxygenated ACSF, and humidified carbogen gas mixture will be directed over the surface of the slice. Baseline temperature will be held at 32° C. Glass recording electrodes filled with 0.9% NaCl and differential amplifiers with a bandpass filter of 1-1,000 Hz at a gain of 1,000 are used. Under high temperature, high extracellular pH, or combined conditions, it is evaluated whether an increase in frequency, amplitude, or altered characteristics of spontaneous field potentials occurs, and whether there is a change in the synaptic components (rate of rise and amplitude), population spike components (amplitude and quantity of spikes) and/or threshold of the evoked field potentials, to determine if high temperature and high pH alter input/output properties.

### Stimulation Protocol:

Electrical stimulation (50 μs duration, 1-100 μA) and varying interpulse intervals of 20-800 msec (Tsai and Leung 2006) are used to measure changes in threshold, amplitude, slope and duration of the second response relative to the first to determine if there is modulation of cellular excitability by inhibitory circuitry.

### Multi-Electrode Array Recordings:

Cyberkinetics, Inc has developed a 96-electrode array system (10×10 with 4 ground electodes) for chronic implantation in human and non-human primates. Electrodes are 1.0 mm long and made of silicone with platinum coated tips. However, its applications in slice electrophysiology have begun to be studied (Song et al. 2004, McCloskey et al. 2007). Coronal slices 400 μm thick are placed in the gas-liquid interface chamber. The array is silicone-bound to a dental brush which is fixed in a Leitz micromanipulator. Once the slice is positioned under the array, the array is slowly lowered into the slice. Once the electrode tips are lowered approximately 200 μm into the slice, threshold settings for each channel are adjusted to optimize spike detection. Various conditions including fixing, re-slicing, staining and enlarging puncture sites are tested in order to extract the maximum amount of information from the preserved tissue. A camera and lens suspended above the interface chamber is used such that pictures of the array and the slice can be taken separately and then digitally aligned to optimize the electrode placement in areas of particular interest, including the hippocampus and neocortex. Using the data acquired to make spatial quantifications is important in providing information regarding propagation patterns.

### Whole-Cell Patch Clamp Recordings:

Slices will be placed in a submersion recording chamber and continuously superfused with aerated ACSF. Micropipettes of 5-12 MΩ are generally filled with (in mM): 135 K-gluconate, 4 KCl, 2 NaCl, 10 HEPES, 0.2 EGTA, 4 ATP-Mg, 0.3 GTP-Tris, 5-10 phosphocreatine-Tris (pH 7.25, 290 mOsm). Intracellular recordings will be made in current-clamp or voltage-clamp mode, as appropriate depending on the experimental aim (Axopatch 1D or Axoclamp 2B). Neurons are visualized with IR-DIC optics using a Zeiss Axioskop and a CCD camera (Hamamatsu). The CA1 region of hippocampus has interneuron types with properties closely similar to those of the FS and LTS cells of neocortex (Pouille & Scanziani, 2001, 2004). To more specifically target interneuron types in the hippocampus, the GIN (Oliva et al. 2000) and G42 lines (Chattopadhyaya et al 2004) mouse lines are used to locate GFP-expressing LTS or FS cell types, respectively, local to the CA1 region. Fluorescent cells are initially located under epifluorescence before switching to IR-DIC for visualized patching. In addition, after patching, each neuron is identified based on its firing properties during constant-current injections 600 ms long, of a range of current intensities (Gibson et al. 1999). Synaptic responses are evoked with extracellular stimuli applied through concentric bipolar microelectrodes (FHC), with pulses lasting 50 μsec and current amplitudes up to 100 RA using a differential amplifier with a low pass filter of 2,000 Hz.

### Quantification of Intrinsic Membrane and Synaptic Properties:

Sub-threshold intrinsic membrane properties are evaluated, resting membrane potentials, input resistance and membrane time constants, at baseline and high temperature and pH conditions by injecting small, incremental negative current steps into the cell. Changes are recorded in the spiking properties, via incremental positive current steps, of these CA1 neurons: action potential threshold, spike amplitude, spike half-width, spike after hyperpolarizations and repetitive spiking patterns during baseline and experimental conditions.

In dual whole-cell recordings, connectivity is tested by injecting short current pulses to elicit action potentials in one cell while recording postsynaptic potentials (PSPs) in the other. This is done bi-directionally. If the patched cells are not found to be chemically or electrically connected in either direction, then one cell will be unpatched. Other adjacent cells will then be patched and tested for connectivity. Tests for spontaneous synaptic events are measured in standard ACSF. Tetrodotoxin is added to block pre-synaptic action potentials, thereby isolating spontaneous fusion events (i.e. miniature PSPs). Changes are segregated in spontaneous excitatory and inhibitory postsynaptic currents (EPSCs and IPSCs respectively) under voltage clamp. To isolate EPSC changes, the membrane is clamped at −70 mV, the reversal potential of IPSCs under these conditions. To isolate IPSC changes, clamping occurs at −65 mV and pipettes are filled with a 30 mM chloride internal solution while blocking AMPA and NMDA receptors, thereby simultaneously eliminating EPSCs and improving IPSC visibility.

### Temperature Modifications and Measurement:

Extracellular field potentials are measured while increasing temperature of ACSF bath from the baseline 34° C. to 40° C. +/−0.5° C. using a TC-102 temperature controller (Medical Systems Corp, Greenvale, N.Y.), facilitated by flushing warm water into the jacket of the base unit (Tancredi et al. 1991). Intracellular recordings are made while increasing temperature of ACSF bath using a TC-324A in-line heater and temperature controller (Warner Instruments, Hamden, Conn.). The readings and rates of temperature increase are monitored and recorded as well as return to baseline in the bath, via miniature thermistor recordings adjacent to the slice during all proposed experiments. pH measurement error due to temperature manipulations is compenstaed for.

### pH Modifications and Measurement:

pH is modified by decreasing P_{CO2} perfusing the artificial cerebral spinal fluid (ACSF) until pH increases 0.2-0.3 units (from 38 mmHg to 15-20 mmHg P_{CO2}) using methods previously described (Eckerman et al. 1990; Schuchmann et al. 2006). pH is monitored in the bath as well as at the slice via pH microelectrode recordings (Voipio and Kaila 1993). This monitoring occurs during all proposed experiments. Differences in pH alteration due to decreasing temperature manipulations in the submerged vs. interface chamber have been reported (Shuchmann et al. 2002). Thus, the importance of monitoring both extracellular pH in both interface and submerged chambers during temperature manipulations is emphasized and will be recorded.

### In Vivo Protocol:

A warm-air induced hyperthermia model developed by Baram et al. (1997) is used Wild-type and TRPV1 knockout mouse littermates at P13-14 are individually placed in a 3 L beaker covered with a donut-shaped Styrofoam lid. A 1600 W Conair 1600 watt hairdryer is placed at an oblique angle above the beaker and warm-air is streamed through the lid's center hole to expose each animal to a hyperthermic environment. Mice are behaviorally monitored for first onset of generalized seizure, usually within 2-4 min of experiment onset. Seizure onset time is recorded. Rectal temperatures are recorded immediately prior to placement in the beaker and to establish baseline and immediately after seizure onset. Seizure threshold temperature and onset time are compared between wild-type and knockout mice.

### TRPV1 In Vivo Pharmacology Experiments:

TRPV1 agonists and antagonists with varying blood-brain barrier permeability will be orally or intraperitoneally administered prior to conducting the in vivo experiment. Seizure onset time and threshold temperature is measured and compared to results obtained without drug manipulations.

### Data Analysis:

Recordings from both the extracellular and intracellular rig are filtered at 10 kHz, digitized, acquired and analyzed using custom software written in Labview. Additional analyses and statistical testing are done in Matlab. Basic parametric statistical tests, including t-tests, analysis of variance and regressions are used where appropriate when making comparison across conditions and/or animals.

### References for Example 1

Al-Hayani, A., Wease, K. N., Ross, R. A., Pertwee, R. G., and Davies, S. N. (2001). The endogenous cannabinoid anandamide activates vanilloid receptors in the rat hippocampal slice. Neuropharmacol 41, 1000-1005.
Audenaert D, Van Broeckhoven C, De Jonghe P (2006) Genes and loci involved in febrile seizures and related epilepsy syndromes. Hum Mutat 27:391-401.
Baram T Z, Gerth A, Schultz L (1997) Febrile seizures: an appropriate-aged model suitable for long-term studies. Brain Res Dev Brain Res 98:265-270.
Beierlein M, Gibson J R, Connors B W (2000) A network of electrically coupled interneurons drives synchronized inhibition in neocortex. Nat Neurosci 3:904-910.
Beierlein M, Gibson J R, Connors B W (2003) Two dynamically distinct inhibitory networks in layer 4 of the neocortex. J Neurophysiol 90:2987-3000.
Benham, C. D., Gunthorpe, M. J., and Davis, J. B. (2003). TRPV channels as temperature sensors. Cell Calcium 33, 479-487.
Caterina, M. J., Schumacher, M. A., Tominaga, M., Rosen, T. A., Levine, J. D., and Julius, D. (1997). The capsaicin receptor: a heat-activated ion channel in the pain pathway. Nature 389, 816-824.
Caterina, M. J., Leffler, A., Malmberg, A. B., Martin, W. J., Trafton, J., Petersen-Zeitz, K. R., Koltzenburg, M., Basbaum, A. I., and Julius, D. (2000). Impaired nociception and pain sensation in mice lacking the capsaicin receptor. Science 288, 306-313.
Chattopadhyaya B, Di Cristo G, Higashiyama H, Knott G W, Kuhlman S J, Welker E, Huang Z J (2004) Experience and activity-dependent maturation of perisomatic GABAergic innervation in primary visual cortex during a postnatal critical period. J Neurosci 24:9598-9611
Chen, K., Neu, A., Howard, A. L., Foldy, C., Echegoyen, J., Hilgenberg, L., Smith, M., Mackie, K., and Soltesz, I. (2007). Prevention of plasticity of endocannabinoid signaling inhibits persistent limbic hyperexcitability caused by developmental seizures. J Neurosci 27, 46-58.
Chevaleyre, V., and Castillo, P. E. (2003). Heterosynaptic LTD of hippocampal GABAergic synapses: a novel role of endocannabinoids in regulating excitability. Neuron 38, 461-472.
Chevaleyre, V., and Castillo, P. E. (2004). Endocannabinoid-mediated metaplasticity in the hippocampus. Neuron 43, 871-881.
Chevaleyre, V., Takahashi, K. A., and Castillo, P. E. (2006). Endocannabinoid-mediated synaptic plasticity in the CNS. Annu Rev Neurosci 29, 37-76.
Cristino, L., de Petrocellis, L., Pryce, G., Baker, D., Guglielmotti, V., and Di Marzo, V. (2006). Immunohistochemical localization of cannabinoid type 1 and vanilloid transient receptor potential vanilloid type 1 receptors in the mouse brain. Neurosci 139, 1405-1415.
Cruikshank S J, Lewis T J, Connors B W (2007) Synaptic basis for intense thalamocortical activation of feedforward inhibitory cells in neocortex. Nat Neurosci 10:462-468.
Cui, M., Honore, P., Zhong, C., Gauvin, D., Mikusa, J., Hernandez, G., Chandran, P., Gomtsyan, A., Brown, B., Bayburt, E. K., et al. (2006). TRPV1 receptors in the CNS play a key role in broad-spectrum analgesia of TRPV1 antagonists. J Neurosci 26, 9385-9393.
De Petrocellis, L., Bisogno, T., Maccarrone, M., Davis, J. B., Finazzi-Agro, A., and Di Marzo, V. (2001). The activity of anandamide at vanilloid VR1 receptors requires facilitated transport across the cell membrane and is limited by intracellular metabolism. J Biol Chem 276, 12856-12863.
De Petrocellis, L., and Di Marzo, V. (2005). Lipids as regulators of the activity of transient receptor potential type V1 (TRPV1) channels. Life Sci 77, 1651-1666.
Deans M R, Gibson J R, Sellitto C, Connors B W, Paul D L (2001) Synchronous activity of inhibitory networks in neocortex requires electrical synapses containing connexin36. Neuron 31:477-485.
del Castillo, J., and Katz, B. (1954). Quantal components of the end-plate potential. J Physiol 124, 560-573.
Dhaka A, Viswanath V, Patapoutian A (2006) Trp ion channels and temperature sensation. Annu Rev Neurosci 29:135-161.
Doly, S., Fischer, J., Salio, C., and Conrath, M. (2004). The vanilloid receptor-1 is expressed in rat spinal dorsal horn astrocytes. Neurosci Lett 357, 123-126.
Dube C, Baram T Z (2005) “Complex” Febrile Seizures—An experimental model in immature rodents. In: Models of Seizures and Epilepsy (Pitkanen A, Schwartzkroin P A, Moshe S L, eds), pp 333-341: Academic Press.
Dube C, Vezzani A, Behrens M, Bartfai T, Baram T Z (2005) Interleukin-1 beta contributes to the generation of experimental febrile seizures. Ann Neurol 57:152-155.
Eckerman P, Scharruhn K, Horowitz J M (1990) Effects of temperature and acid-base state on hippocampal population spikes in hamsters. Am J Physiol 258:R1140-1146.
Feinmark, S. J., Begum, R., Tsvetkov, E., Goussakov, I., Funk, C. D., Siegelbaum, S. A., and Bolshakov, V. Y. (2003). 12-lipoxygenase metabolites of arachidonic acid mediate metabotropic glutamate receptor-dependent long-term depression at hippocampal CA3-CA1 synapses. J Neurosci 23, 11427-11435.
Ferraguti, F., Cobden, P., Pollard, M., Cope, D., Shigemoto, R., Watanabe, M., and Somogyi, P. (2004). Immunolocalization of metabotropic glutamate receptor 1alpha (mGluR1alpha) in distinct classes of interneuron in the CA1 region of the rat hippocampus. Hippocampus 14, 193-215.
Freund, T. F., and Buzsaki, G. (1996). Interneurons of the hippocampus. Hippocampus 6, 347-470.
Gerdeman, G. L., Ronesi, J., and Lovinger, D. M. (2002). Postsynaptic endocannabinoid release is critical to long-term depression in the striatum. Nat Neurosci 5, 446-451.
Gibson J R, Beierlein M, Connors B W (1999) Two networks of electrically coupled inhibitory neurons in neocortex. Nature 402:75-79.
Gulyas, A. I., Toth, K., McBain, C. J., and Freund, T. F. (1998). Stratum radiatum giant cells: a type of principal cell in the rat hippocampus. Eur J Neurosci 10, 3813-3822.
Hajos, N., and Freund, T. F. (2002). Pharmacological separation of cannabinoid sensitive receptors on hippocampal excitatory and inhibitory fibers. Neuropharmacol 43, 503-510.
Holtzman D, Obana K, Olson J (1981) Hyperthermia-induced seizures in the rat pup: a model for febrile convulsions in children. Science 213:1034-1036.
Holzer, P. (1988). Local effector functions of capsaicin-sensitive sensory nerve endings: involvement of tachykinins, calcitonin gene-related peptide and other neuropeptides. Neurosci 24, 739-768.
Hu D E, Easton A S, Fraser P A (2005) TRPV1 activation results in disruption of the blood-brain barrier in the rat. Br J Pharmacol 146:576-584.
Huang, S. M., Bisogno, T., Trevisani, M., Al-Hayani, A., De Petrocellis, L., Fezza, F., Tognetto, M., Petros, T. J., Krey, J. F., Chu, C. J., et al. (2002). An endogenous capsaicin-like substance with high potency at recombinant and native vanilloid VR1 receptors. Proc Natl Acad Sci USA 99, 8400-8405.
Hwang, S. W., Cho, H., Kwak, J., Lee, S. Y., Kang, C. J., Jung, J., Cho, S., Min, K. H., Suh, Y. G., Kim, D., and Oh, U. (2000). Direct activation of capsaicin receptors by products of lipoxygenases: endogenous capsaicin-like substances. Proc Natl Acad Sci USA 97, 6155-6160.
Jordt, S. E., and Julius, D. (2002). Molecular basis for species-specific sensitivity to “hot” chili peppers. Cell 108, 421-430.
Kim, S. R., Kim, S. U., Oh, U., and Jin, B. K. (2006). Transient receptor potential vanilloid subtype 1 mediates microglial cell death in vivo and in vitro via Ca2+-mediated mitochondrial damage and cytochrome c release. J Immunol 177, 4322-4329.
Kreitzer, A. C., and Regehr, W. G. (2001). Retrograde inhibition of presynaptic calcium influx by endogenous cannabinoids at excitatory synapses onto Purkinje cells. Neuron 29, 717-727.
Kreitzer, A. C., and Malenka, R. C. (2005). Dopamine modulation of state-dependent endocannabinoid release and long-term depression in the striatum. J Neurosci 25, 10537-10545.
Kullmann, D. M., and Lamsa, K. P. (2007). Long-term synaptic plasticity in hippocampal interneurons. Nat Rev Neurosci 8, 687-699.
Lei, S., and McBain, C. J. (2004). Two Loci of expression for long-term depression at hippocampal mossy fiber-interneuron synapses. J Neurosci 24, 2112-2121.
Lipski, J., Park, T. I., Li, D., Lee, S. C., Trevarton, A. J., Chung, K. K., Freestone, P. S., and Bai, J. Z. (2006). Involvement of TRP-like channels in the acute ischemic response of hippocampal CA1 neurons in brain slices. Brain Res 1077, 187-199.
Llano, I., Leresche, N., and Marty, A. (1991). Calcium entry increases the sensitivity of cerebellar Purkinje cells to applied GABA and decreases inhibitory synaptic currents. Neuron 6, 565-574.
Lowenstein D N (2005) Seizures and Epilepsy. In: Harrison's Principles of Internal Medicine (Kasper D L, Harrison T R, eds), pp 2357-2372. New York, N.Y.: McGraw-Hill.
Maejima, T., Hashimoto, K., Yoshida, T., Aiba, A., and Kano, M. (2001). Presynaptic inhibition caused by retrograde signal from metabotropic glutamate to cannabinoid receptors. Neuron 31, 463-475.
Malenka, R. C., and Bear, M. F. (2004). LTP and LTD: an embarrassment of riches. Neuron 44, 5-21.
Malinow, R., and Tsien, R. W. (1990). Presynaptic enhancement shown by whole-cell recordings of long-term potentiation in hippocampal slices. Nature 346, 177-180.
Manabe, T., Renner, P., and Nicoll, R. A. (1992). Postsynaptic contribution to long-term potentiation revealed by the analysis of miniature synaptic currents. Nature 355, 50-55.
Mann, E. O., and Paulsen, O. (2007). Role of GABAergic inhibition in hippocampal network oscillations. Trends Neurosci 30, 343-349.
Marinelli, S., Di Marzo, V., Berretta, N., Matias, I., Maccarrone, M., Bernardi, G., and Mercuri, N. B. (2003). Presynaptic facilitation of glutamatergic synapses to dopaminergic neurons of the rat substantia nigra by endogenous stimulation of vanilloid receptors. J Neurosci 23, 3136-3144.
Marinelli, S., Di Marzo, V., Florenzano, F., Fezza, F., Viscomi, M. T., van der Stelt, M., Bernardi, G., Molinari, M., Maccarrone, M., and Mercuri, N. B. (2007). N-arachidonoyl-dopamine tunes synaptic transmission onto dopaminergic neurons by activating both cannabinoid and vanilloid receptors. Neuropsychopharmacol 32, 298-308.
Marsch, R., Foeller, E., Rammes, G., Bunck, M., Kossl, M., Holsboer, F., Zieglgansberger, W., Landgraf, R., Lutz, B., and Wotjak, C. T. (2007). Reduced anxiety, conditioned fear, and hippocampal long-term potentiation in transient receptor potential vanilloid type 1 receptor-deficient mice. J Neurosci 27, 832-839.
Matta, J. A., Miyares, R. L., and Ahern, G. P. (2007). TRPV1 is a novel target for omega-3 polyunsaturated fatty acids. J Physiol 578, 397-411.
McCloskey D (2007) Spontaneous epileptiform burst discharges in the epileptic and control rat hippocampal slice recorded with a 96 tip multielectrode array. In: Epilepsy: From Cellular to Network Mechanisms. Society for Neuroscience Conference-San Diego, Calif.: Helen Hayes Hospital.
McMahon, L. L., and Kauer, J. A. (1997). Hippocampal interneurons express a novel form of synaptic plasticity. Neuron 18, 295-305.
Mezey, E., Toth, Z. E., Cortright, D. N., Arzubi, M. K., Krause, J. E., Elde, R., Guo, A., Blumberg, P. M., and Szallasi, A. (2000). Distribution of mRNA for vanilloid receptor subtype 1 (VR1), and VR1-like immunoreactivity, in the central nervous system of the rat and human. Proc Natl Acad Sci USA 97, 3655-3660.
O'Sullivan, S. E., Kendall, D. A., and Randall, M. D. (2004). Characterisation of the vasorelaxant properties of the novel endocannabinoid N-arachidonoyl-dopamine (NADA). Br J Pharmacol 141, 803-812.
Ohno-Shosaku, T., Maejima, T., and Kano, M. (2001). Endogenous cannabinoids mediate retrograde signals from depolarized postsynaptic neurons to presynaptic terminals. Neuron 29, 729-738.
Oliva A A, Jr., Jiang M, Lam T, Smith K L, Swann J W (2000) Novel hippocampal interneuronal subtypes identified using transgenic mice that express green fluorescent protein in GABAergic interneurons. J Neurosci 20:3354-3368.
Padwal, R. S., and Majumdar, S. R. (2007). Drug treatments for obesity: orlistat, sibutramine, and rimonabant. Lancet 369, 71-77.
Parra, P., Gulyas, A. I., and Miles, R. (1998). How many subtypes of inhibitory cells in the hippocampus? Neuron 20, 983-993.
Patapoutian A, Peier A M, Story G M, Viswanath V (2003) ThermoTRP channels and beyond: mechanisms of temperature sensation. Nat Rev Neurosci 4:529-539.
Pegorini, S., Zani, A., Braida, D., Guerini-Rocco, C., and Sala, M. (2006). Vanilloid VR1 receptor is involved in rimonabant-induced neuroprotection. Br J Pharmacol 147, 552-559.
Pitler, T. A., and Alger, B. E. (1992). Postsynaptic spike firing reduces synaptic GABAA responses in hippocampal pyramidal cells. J Neurosci 12, 4122-4132.
Pouille F, Scanziani M (2001) Enforcement of temporal fidelity in pyramidal cells by somatic feed-forward inhibition. Science 293:1159-1163.
Pouille F, Scanziani M (2004) Routing of spike series by dynamic circuits in the hippocampus. Nature 429:717-723.
Robbe, D., Kopf, M., Remaury, A., Bockaert, J., and Manzoni, O. J. (2002). Endogenous cannabinoids mediate long-term synaptic depression in the nucleus accumbens. Proc Natl Acad Sci USA 99, 8384-8388.
Roberts, J. C., Davis, J. B., and Benham, C. D. (2004). [3H]Resiniferatoxin autoradiography in the CNS of wild-type and TRPV1 null mice defines TRPV1 (VR-1) protein distribution. Brain Res 995, 176-183.
Ronesi, J., Gerdeman, G. L., and Lovinger, D. M. (2004). Disruption of endocannabinoid release and striatal long-term depression by postsynaptic blockade of endocannabinoid membrane transport. J Neurosci 24, 1673-1679.
Sanchez, J. F., Krause, J. E., and Cortright, D. N. (2001). The distribution and regulation of vanilloid receptor VR1 and VR1 5′ splice variant RNA expression in rat. Neurosci 107, 373-381.
Sasamura, T., Sasaki, M., Tohda, C., and Kuraishi, Y. (1998). Existence of capsaicin-sensitive glutamatergic terminals in rat hypothalamus. Neuroreport 9, 2045-2048.
Schuchmann S, Schmitz D, Rivera C, Vanhatalo S, Salmen B, Mackie K, Sipila S T, Voipio J, Kaila K (2006) Experimental febrile seizures are precipitated by a hyperthermia-induced respiratory alkalosis. Nat Med 12:817-823.
Shibasaki, K., Suzuki, M., Mizuno, A., and Tominaga, M. (2007). Effects of body temperature on neural activity in the hippocampus: regulation of resting membrane potentials by transient receptor potential vanilloid 4. J Neurosci 27, 1566-1575.
Shin, J., Cho, H., Hwang, S. W., Jung, J., Shin, C. Y., Lee, S. Y., Kim, S. H., Lee, M. G., Choi, Y. H., Kim, J., et al. (2002). Bradykinin-12-lipoxygenase-VR1 signaling pathway for inflammatory hyperalgesia. Proc Natl Acad Sci USA 99, 10150-10155.
Singla, S., Kreitzer, A. C., and Malenka, R. C. (2007). Mechanisms for synapse specificity during striatal long-term depression. J Neurosci 27, 5260-5264.
Sjostrom, P. J., Turrigiano, G. G., and Nelson, S. B. (2003). Neocortical LTD via coincident activation of presynaptic NMDA and cannabinoid receptors. Neuron 39, 641-654.
Smart, D., Gunthorpe, M. J., Jerman, J. C., Nasir, S., Gray, J., Muir, A. I., Chambers, J. K., Randall, A. D., and Davis, J. B. (2000). The endogenous lipid anandamide is a full agonist at the human vanilloid receptor (hVR1). Br J Pharmacol 129, 227-230.
Sohn, J. W., Lee, D., Cho, H., Lim, W., Shin, H. S., Lee, S. H., and Ho, W. K. (2007). Receptor-specific inhibition of GABAB-activated K+ currents by muscarinic and metabotropic glutamate receptors in immature rat hippocampus. J Physiol 580, 411-422.
Song Y K, Patterson W, Bull C, Hwang N J, Deangelis A, Lay C, Connors B, McKay J, Nurmikko A, Donoghue J (2004) Development of an integrated microelectrode/microelectronic device for brain implantable neuroengineering applications. Conf Proc IEEE Eng Med Biol Soc 6:4053-4056.
Srinivasan J, Wallace K A, Scheffer I E (2005) Febrile seizures. Aust Fam Physician 34:1021-1025.
Stafstrom C E (2002) Incidence and prevalence of febrile seizures. In: Febrile Seizures (Baram T Z, Shinnar S, eds), pp 169-188. San Diego, Calif.: Academic Press.
Steenland, H. W., Ko, S. W., Wu, L. J., and Zhuo, M. (2006). Hot receptors in the brain. Mol Pain 2, 34.
Szabo, T., Biro, T., Gonzalez, A. F., Palkovits, M., and Blumberg, P. M. (2002). Pharmacological characterization of vanilloid receptor located in the brain. Brain Res Mol Brain Res 98, 51-57.
Szallasi, A., and Blumberg, P. M. (1999). Vanilloid (Capsaicin) receptors and mechanisms. Pharmacol Rev 51, 159-212.
Szallasi, A., and Appendino, G. (2004). Vanilloid receptor TRPV1 antagonists as the next generation of painkillers. Are we putting the cart before the horse? J Med Chem 47, 2717-2723.
Szallasi, A., Cruz, F., and Geppetti, P. (2006). TRPV1: a therapeutic target for novel analgesic drugs? Trends Mol Med 12, 545-554.
Takahashi, K. A., and Castillo, P. E. (2006). The CB1 cannabinoid receptor mediates glutamatergic synaptic suppression in the hippocampus. Neurosci 139, 795-802.
Tancredi V, D'Arcangelo G, Zona C, Siniscalchi A, Avoli M (1992) Induction of epileptiform activity by temperature elevation in hippocampal slices from young rats: an in vitro model for febrile seizures? Epilepsia 33:228-234.
Tominaga, M., Caterina, M. J., Malmberg, A. B., Rosen, T. A., Gilbert, H., Skinner, K., Raumann, B. E., Basbaum, A. I., and Julius, D. (1998). The cloned capsaicin receptor integrates multiple pain-producing stimuli. Neuron 21, 531-543.
Tominaga M, Tominaga T (2005) Structure and function of TRPV1. Pflugers Arch 451:143-150.
Toth, A., Boczan, J., Kedei, N., Lizanecz, E., Bagi, Z., Papp, Z., Edes, I., Csiba, L., and Blumberg, P. M. (2005). Expression and distribution of vanilloid receptor 1 (TRPV1) in the adult rat brain. Brain Res Mol Brain Res 135, 162-168.
Tsai M L, Leung L S (2006) Decrease of hippocampal GABA B receptor-mediated inhibition after hyperthermia-induced seizures in immature rats. Epilepsia 47:277-287.
Tucci, S. A., Halford, J. C., Harrold, J. A., and Kirkham, T. C. (2006). Therapeutic potential of targeting the endocannabinoids: implications for the treatment of obesity, metabolic syndrome, drug abuse and smoking cessation. Curr Med Chem 13, 2669-2680.
Van Der Stelt, M., and Di Marzo, V. (2004). Endovanilloids. Putative endogenous ligands of transient receptor potential vanilloid 1 channels. Eur J Biochem 271, 1827-1834.
Vellani, V., Mapplebeck, S., Moriondo, A., Davis, J. B., and McNaughton, P. A. (2001). Protein kinase C activation potentiates gating of the vanilloid receptor VR1 by capsaicin, protons, heat and anandamide. J Physiol 534, 813-825.
Voipio J, Kaila K (1993) Interstitial PCO2 and pH in rat hippocampal slices measured by means of a novelfast CO2/H(+)-sensitive microelectrode based on a PVC-gelled membrane. Pflugers Arch 423:193-201.
Waruiru C, Appleton R (2004) Febrile seizures: an update. Arch Dis Child 89:751-756.
Wilson, R. I., and Nicoll, R. A. (2001). Endogenous cannabinoids mediate retrograde signalling at hippocampal synapses. Nature 410, 588-592.
Wu, Z. Z., Chen, S. R., and Pan, H. L. (2005). Transient receptor potential vanilloid type 1 activation down-regulates voltage-gated calcium channels through calcium-dependent calcineurin in sensory neurons. J Biol Chem 280, 18142-18151.
Wu, Z. Z., Chen, S. R., and Pan, H. L. (2006). Signaling mechanisms of down-regulation of voltage-activated Ca2+ channels by transient receptor potential vanilloid type 1 stimulation with olvanil in primary sensory neurons. Neurosci 141, 407-419.
Zygmunt, P. M., Petersson, J., Andersson, D. A., Chuang, H., Sorgard, M., Di Marzo, V., Julius, D., and Hogestatt, E. D. (1999). Vanilloid receptors on sensory nerves mediate the vasodilator action of anandamide. Nature 400, 452-457.

### Example 2

### Heat-Activated TRPV1 Channels Excite Hippocampal Neurons and Enhance Susceptibility to Febrile Seizures

The developing brain is particularly susceptible to adverse events. Febrile seizures are the most prevalent type of seizure among young children, yet their underlying mechanisms remain elusive. High brain temperature alone is sufficient to induce developmentally regulated seizures (Holtzman et al., 1981; Tancredi et al. 1992; Baram et al., 1997; Schuchmann et al., 2006), suggesting that some elements of the immature brain are particularly heat-sensitive. In the peripheral nervous system, TRPV1 channels are heat-sensitive cation channels (Caterina et al., 2000) that confer steep temperature-sensitivity upon primary sensory afferents (Dhaka et al., 2006). Recent studies have implicated the TRPV family of channels in the regulation of central neuron excitability, plasticity, and susceptibility to injury (Gibson et al., 2008; Shibasaki et al., 2007; Lipski et al., 2006; Huang et al., 2002; Al-Hayani et al., 2001; Hajos & Freund, 2002; Kauer & Gibson, in press). The expression of these temperature-sensitive channels in the brain suggests that they may contribute to the susceptibility to hyperthermic seizures. Here we show that TRPV1 channels directly increase the heat-triggered excitability of hippocampal neurons at temperatures within physiological and febrile ranges, and that TRPV1 channels enhance the brain's susceptibility to hyperthermic seizures in vivo.

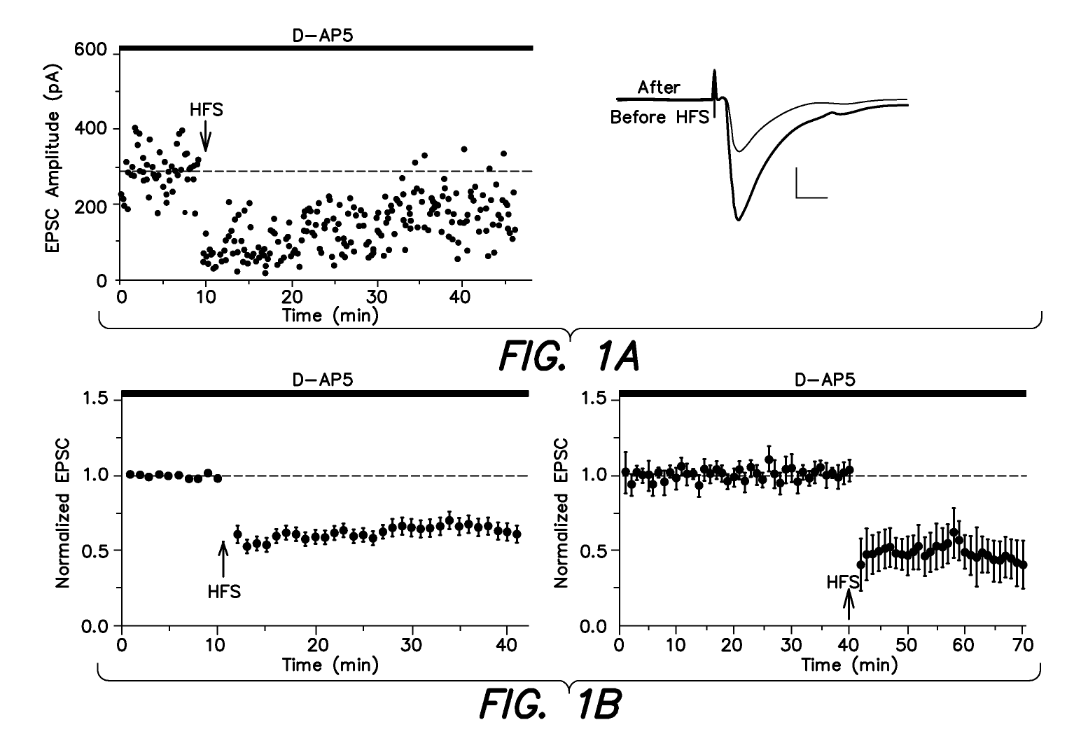

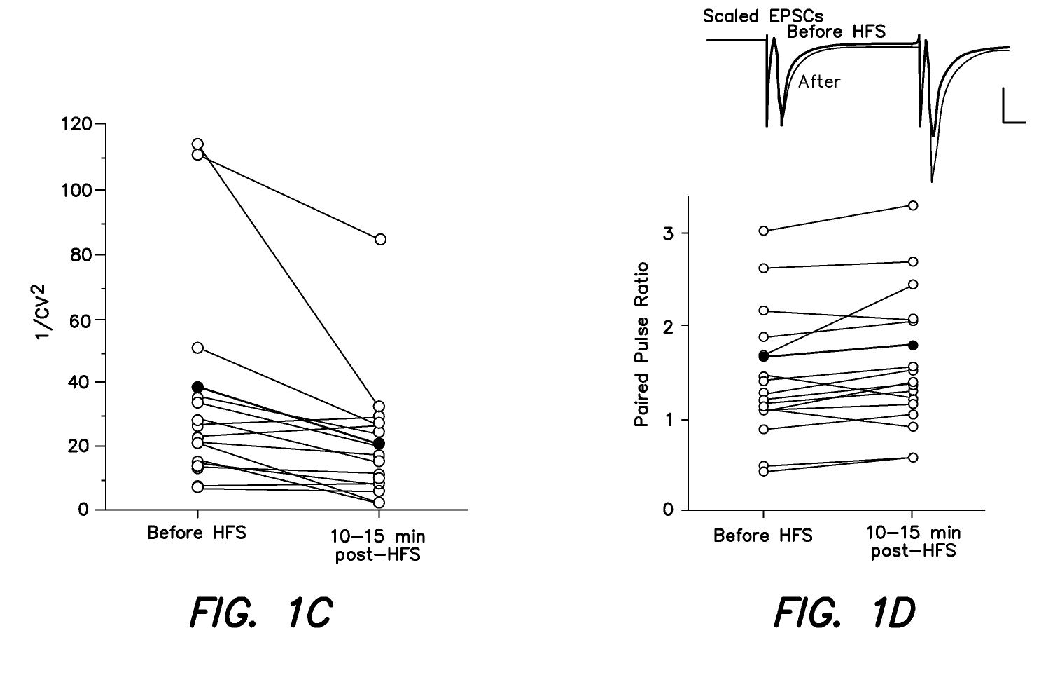

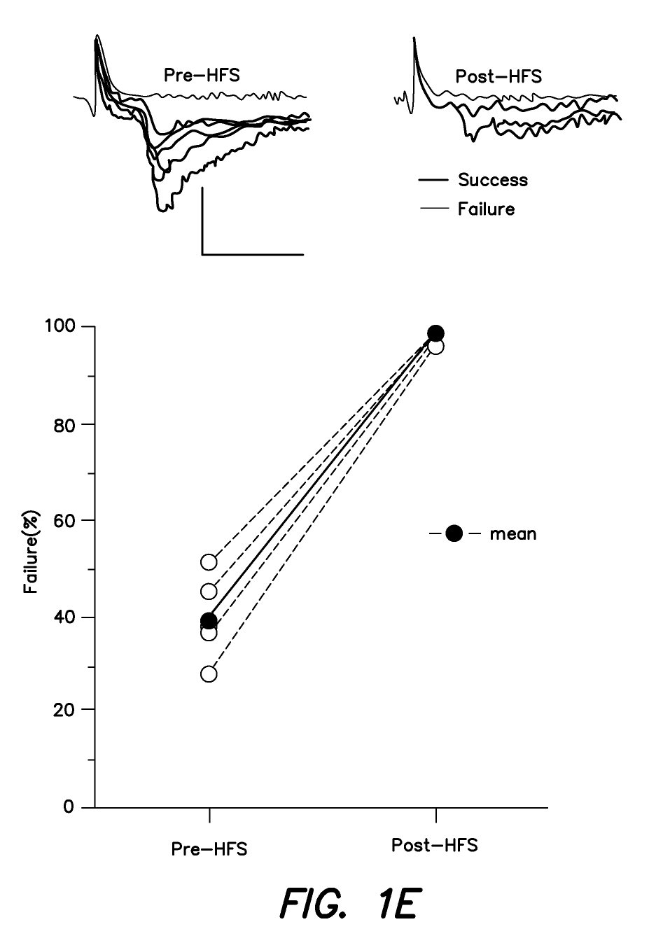

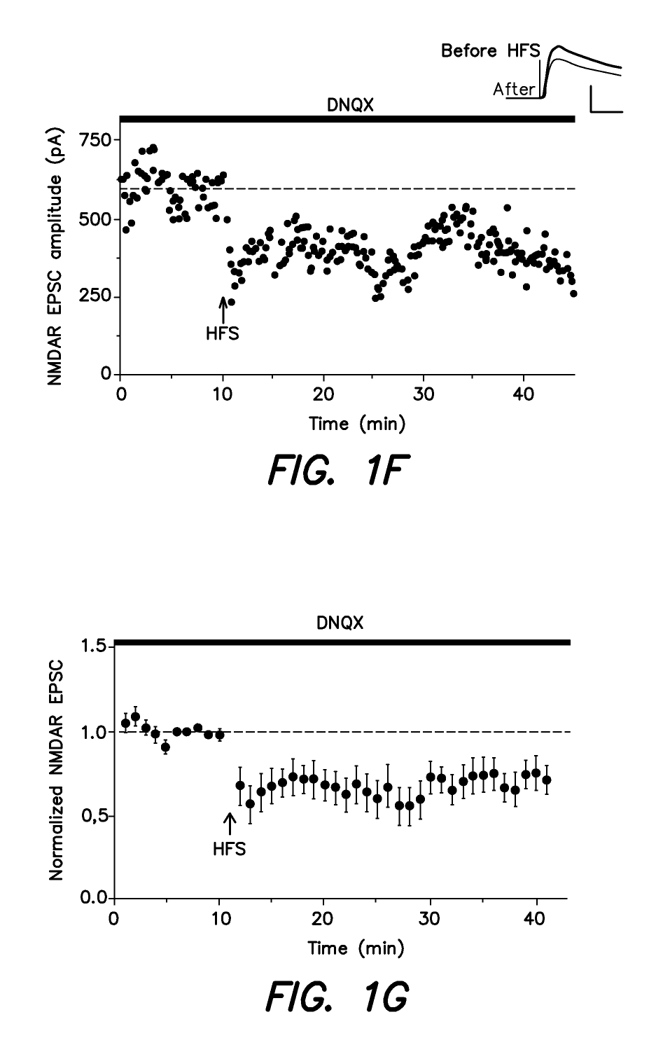

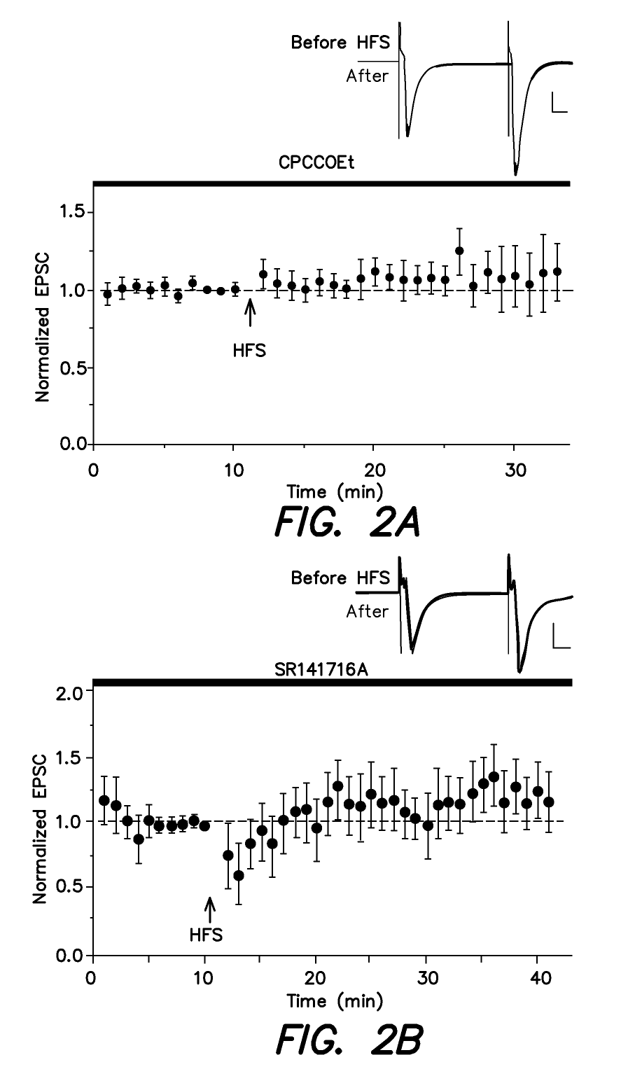

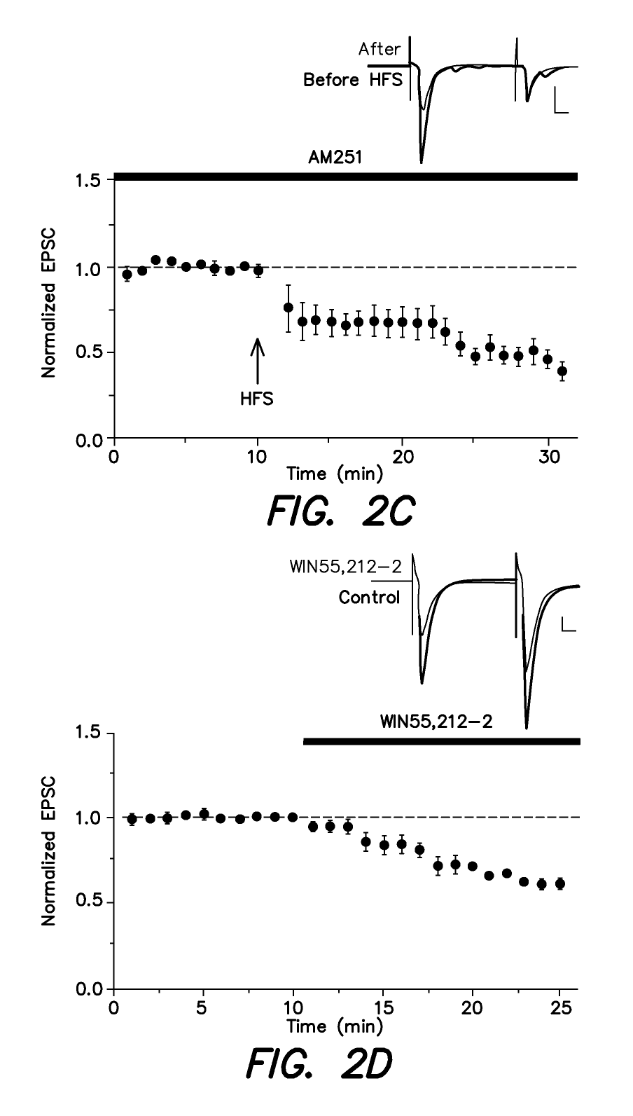

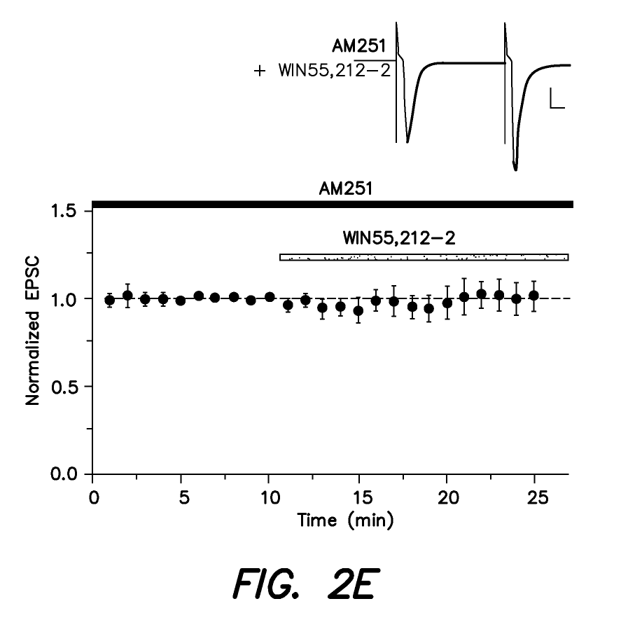

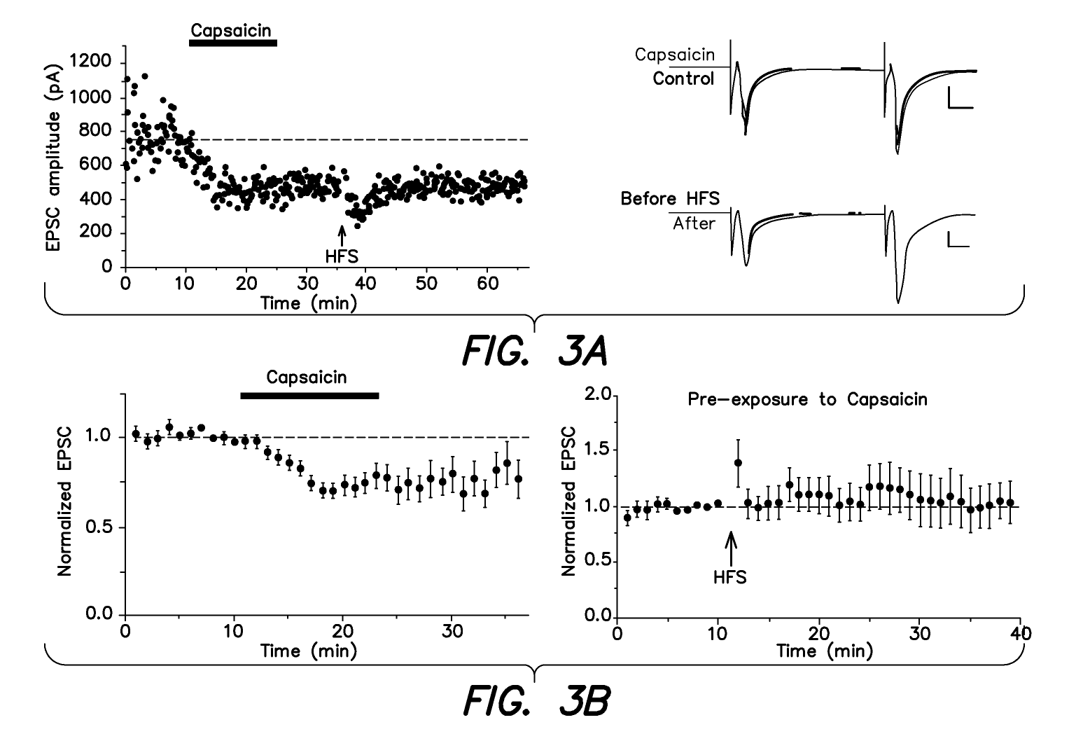

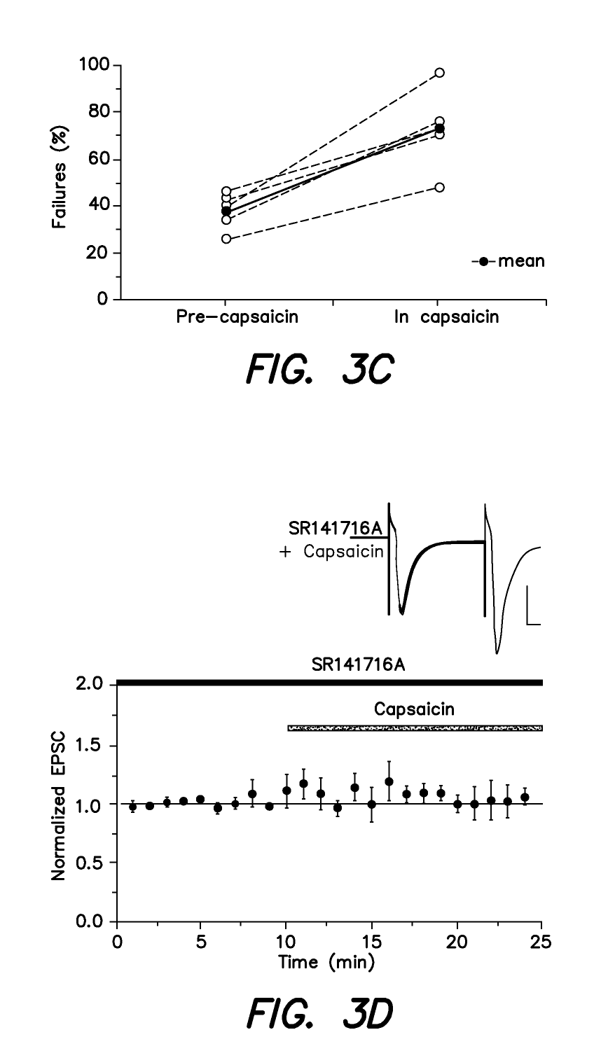

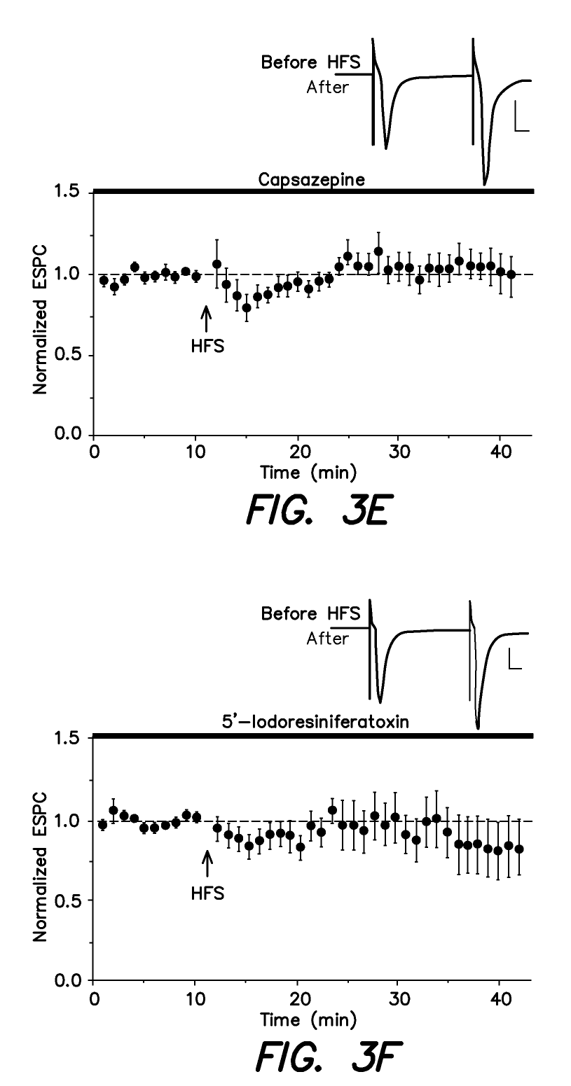

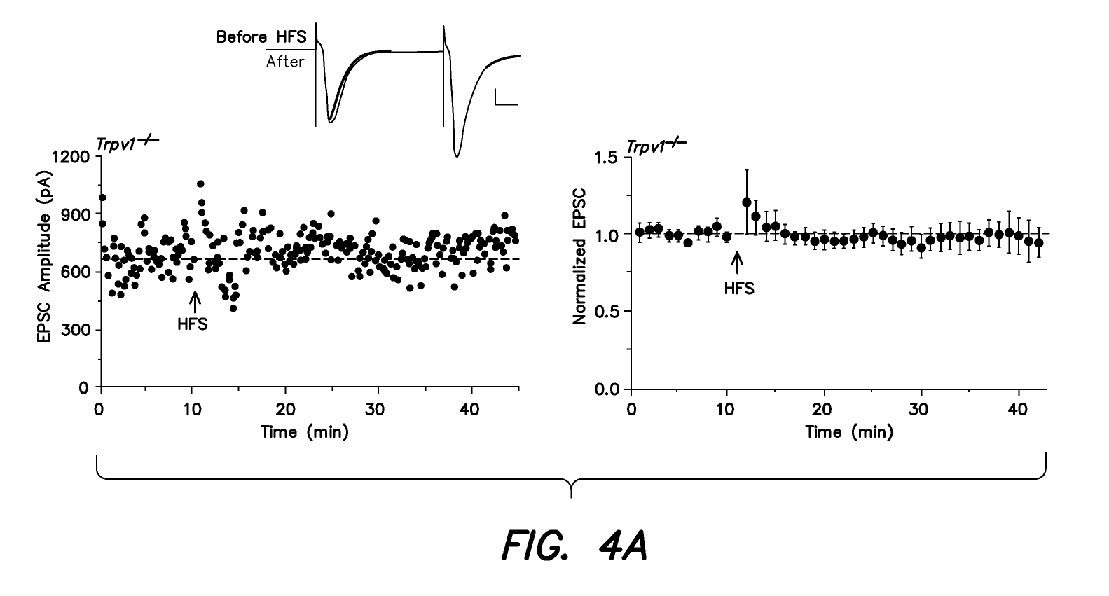

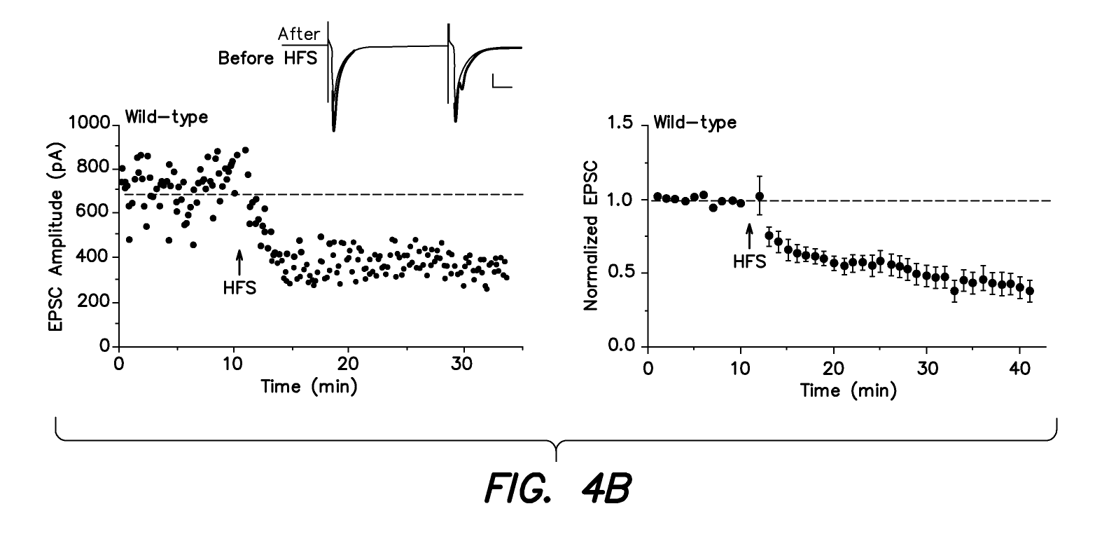

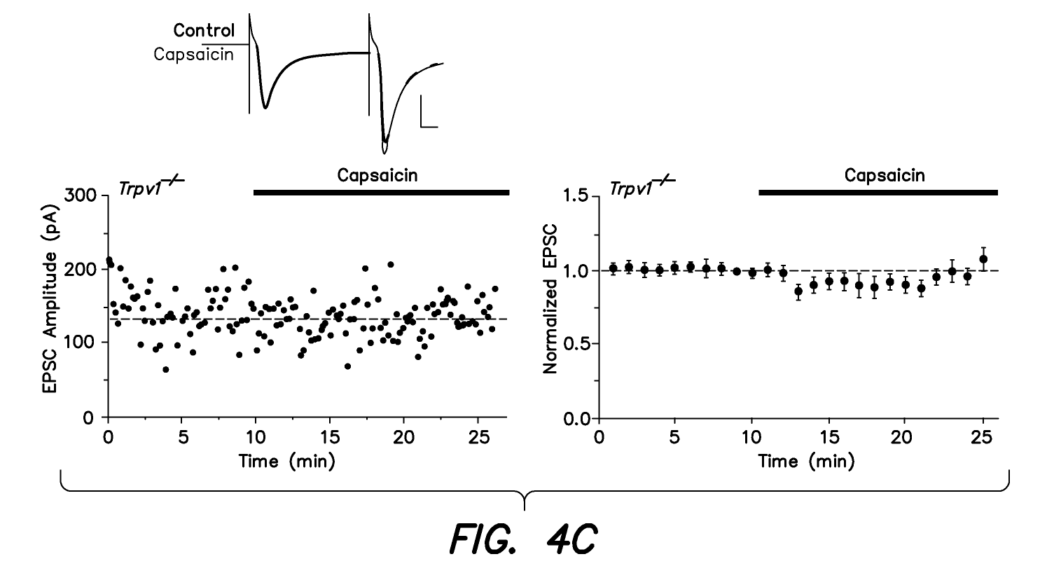

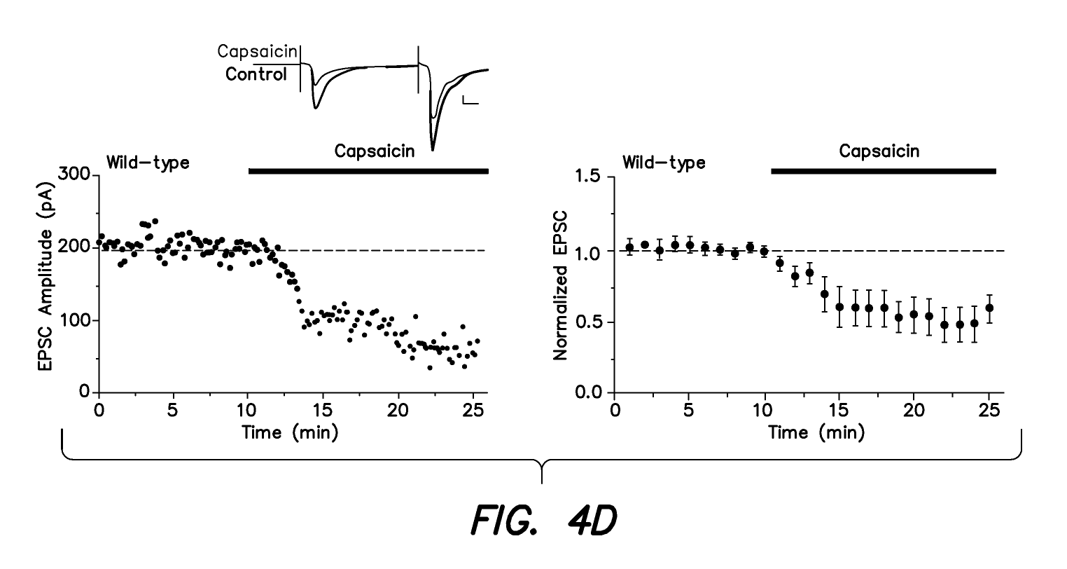

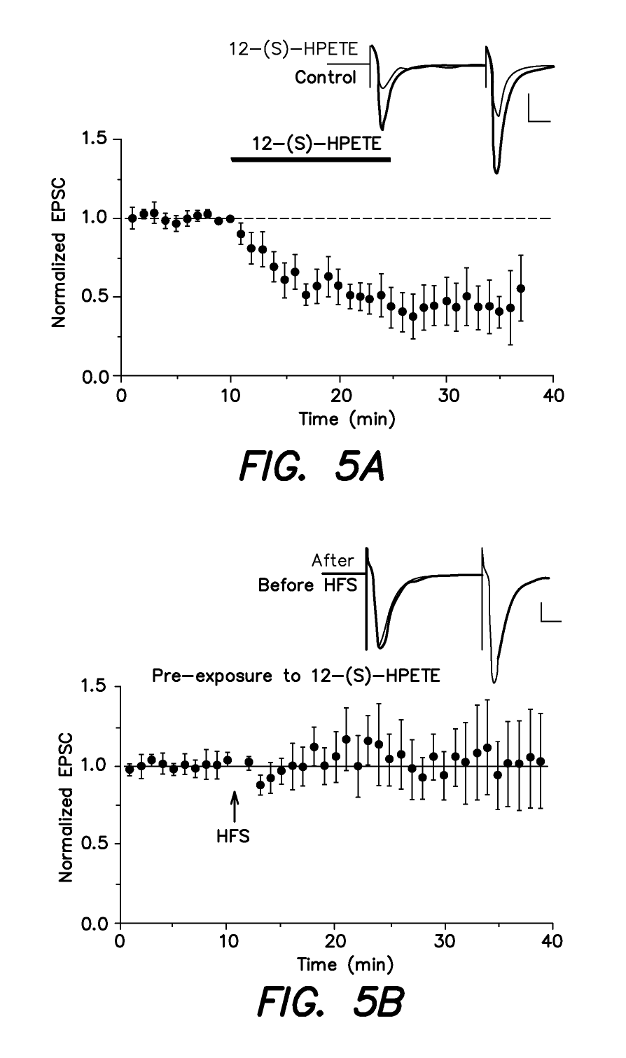

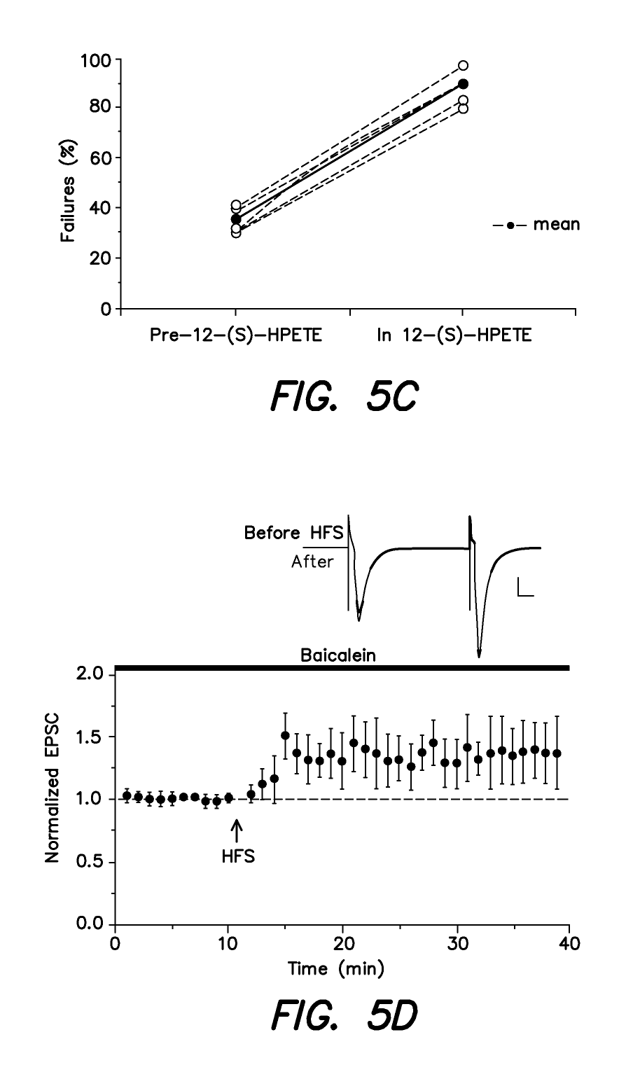

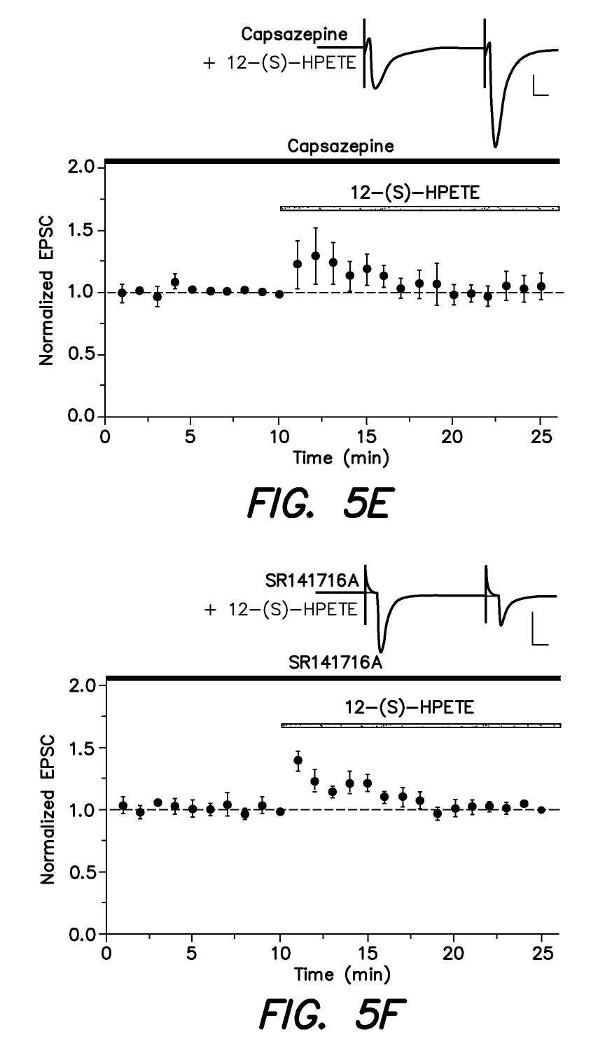

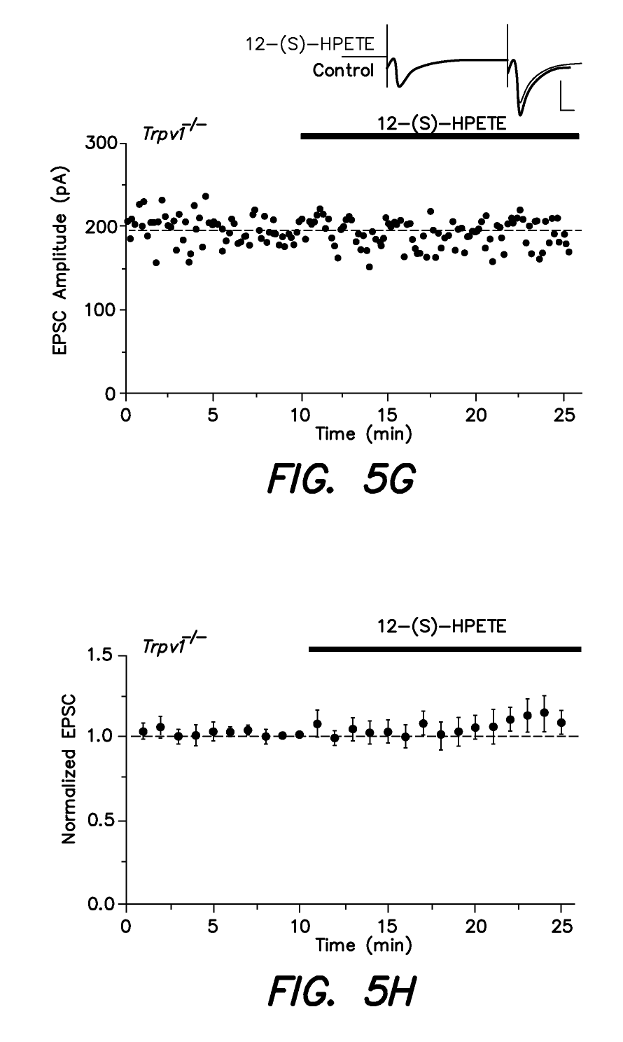

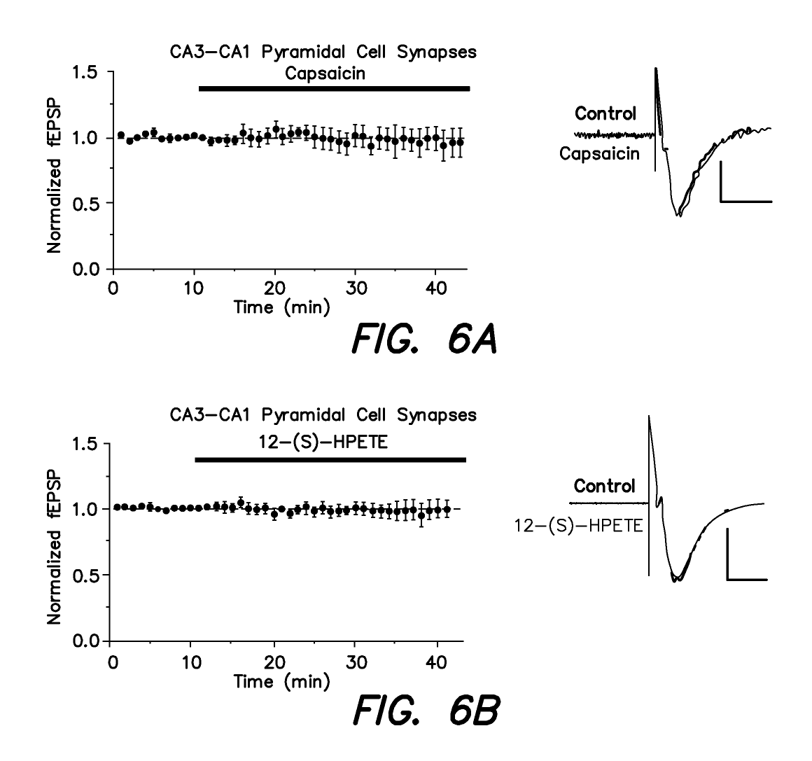

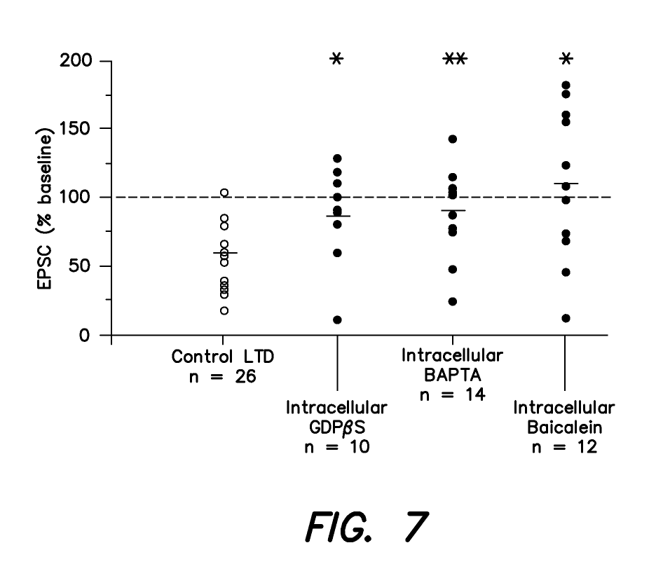

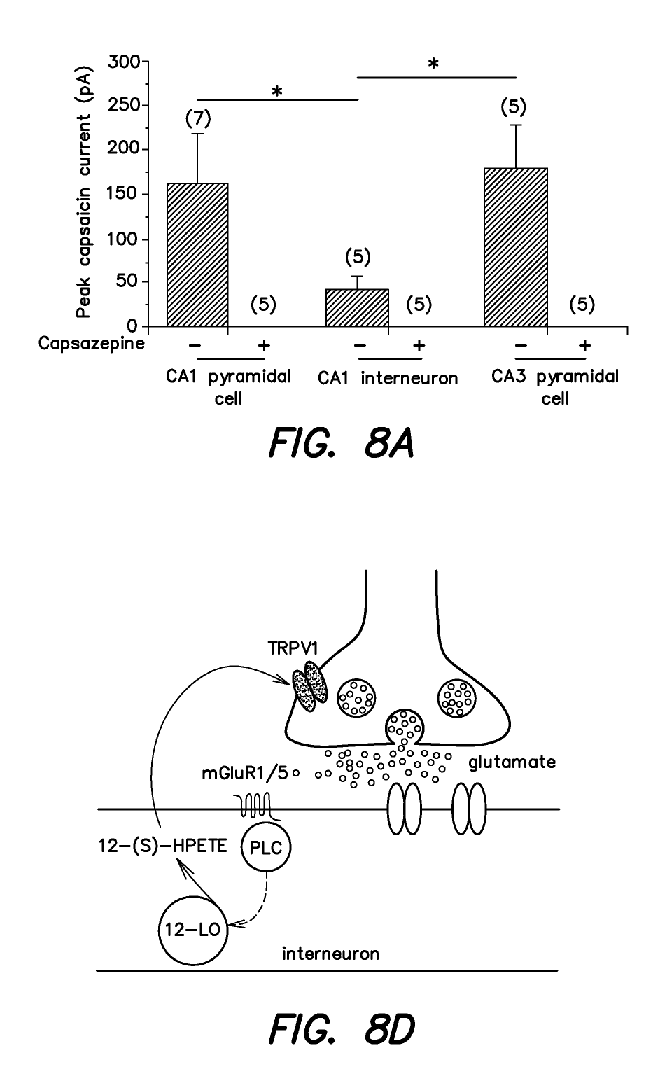

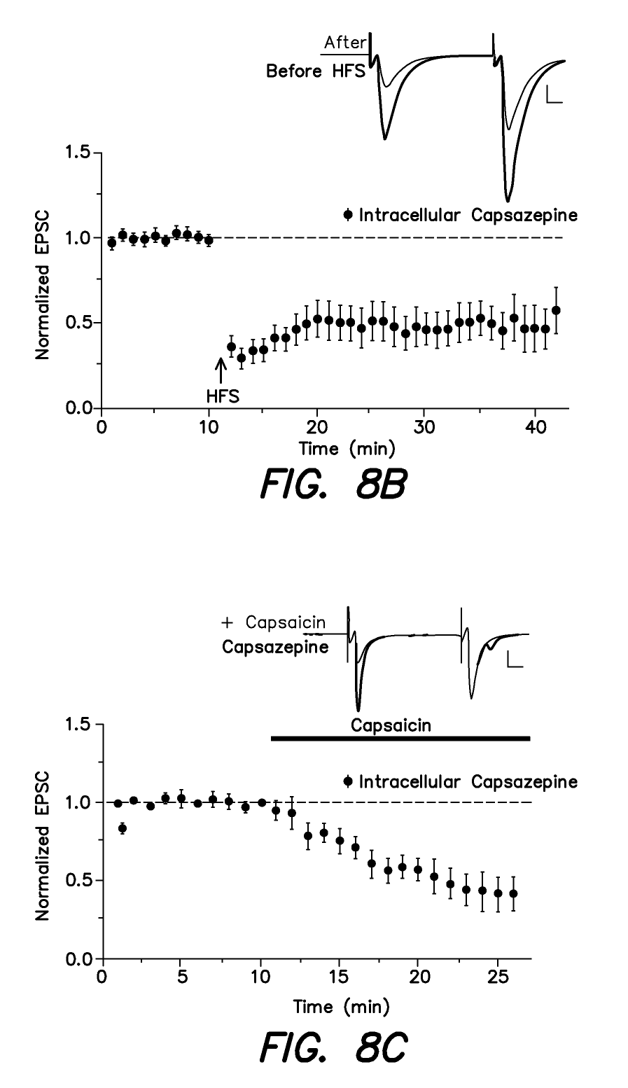

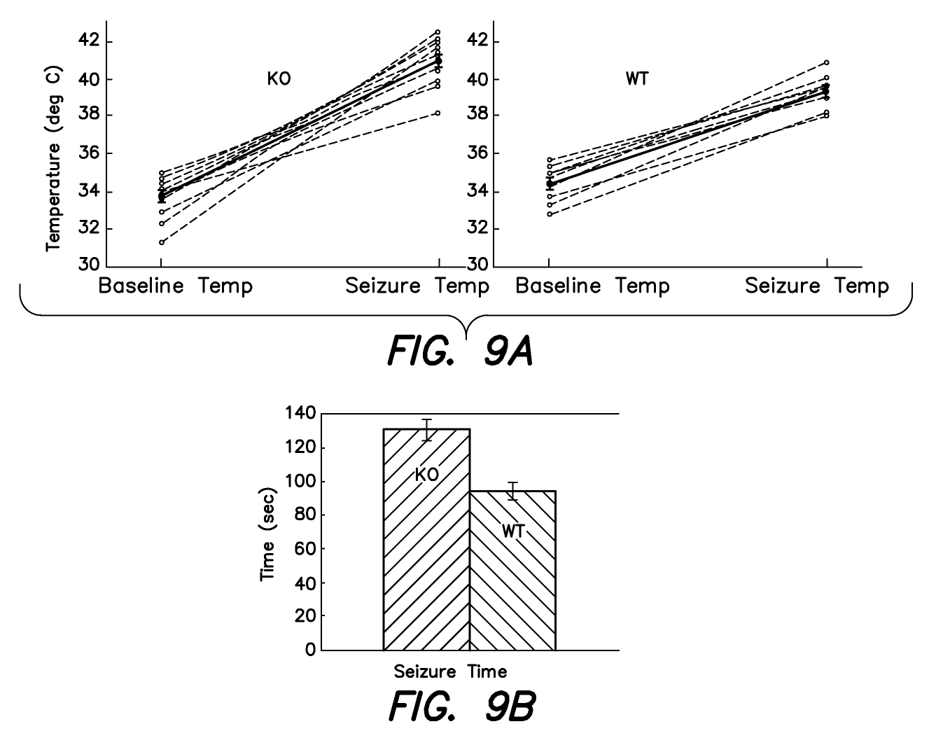

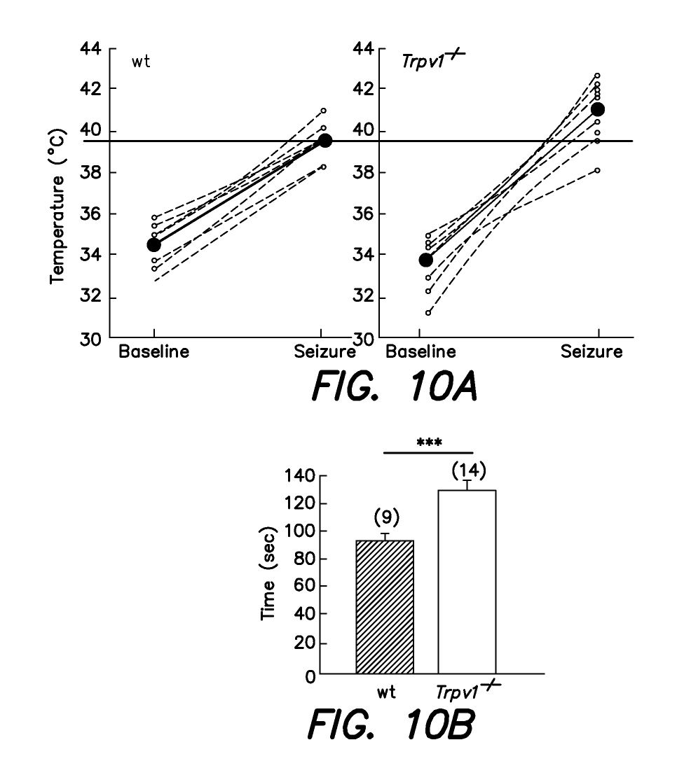

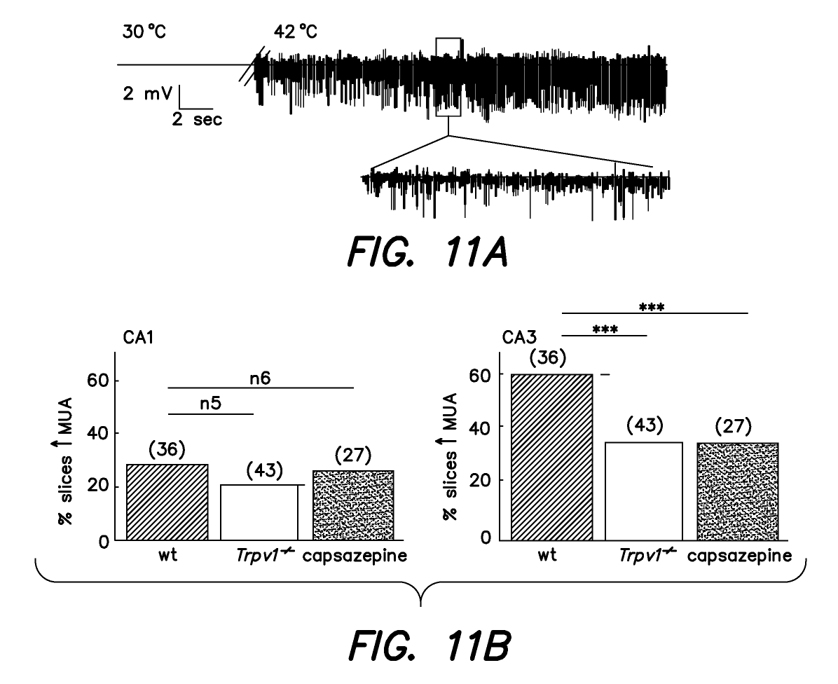

To establish a relationship between TRPV1 channel activation and febrile seizures, we first tested whether TRPV1 channels render an animal more susceptible to hyperthermic seizures. We induced hyperthermic seizures in vivo by raising the core body temperature of immature wild-type and trpv1^{-/-} mice. Febrile seizures were induced in wild-type mice at a mean temperature of 39.5° C., whereas seizure thresholds in trpv1^{-/-} mice were significantly higher at 41.1° C. (FIG. 10a). Baseline temperatures in the two genotypes were not different. The time to seizure onset was also significantly delayed in trpv1^{-/-} mice as compared to wild-type mice (FIG. 10b). These results demonstrate that the presence of TRPV1 channels significantly increases hyperthermic seizure susceptibility in vivo.
TRPV1 channels are expressed in central neurons (Caterina et al., 2000; Kauer & Gibson, in press; Toth et al., 2005), including pyramidal cells of the hippocampus. To determine whether centrally located TRPV1 channels promote temperaturedependent excitability, we recorded spontaneous activity in stratum pyramidale of the CA1 and CA3 regions of hippocampal slices from trpv1^{-/-} and wild-type mice. Heating induced an increase in the frequency of action potentials and field potential bursts in slices from both genotypes (FIGS. 11a and b). Although the site of origin for febrile seizures is unknown (Baram et al., 1997; Mitchell & Lewis, 2002), the recurrent connections of CA3 pyramidal cells make this area highly seizure-prone (Spruston & McBain, 2007; Prince & Connors, 1986). While the percentage of slices exhibiting increased multi-unit activity (MUA) in CA1 did not differ significantly between trpv1^{-/-} and wild-type slices (FIG. 11b), in area CA3 fewer trpv1^{-/-} slices showed an increase in multi-unit activity as compared to wild-type slices (FIG. 11b). Pre-treating wild-type slices with TRPV1 receptor antagonist, capsazepine, mimicked trpv1^{-/-} incidence rates of increased MUA in area CA3 (FIG. 2b). The normalized amplitude and time-course of increased MUA were unaffected (FIG. 14). Our data suggest that TRPV1 channels contribute both to temperature-triggered seizure susceptibility in vivo and to increased temperature-dependent excitability in vitro.

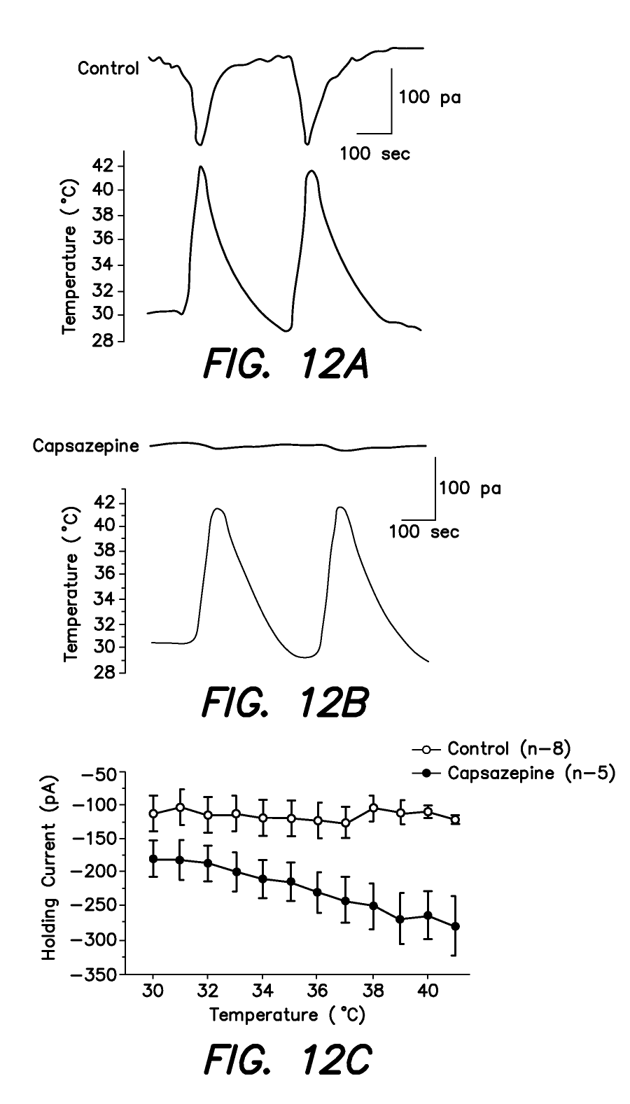

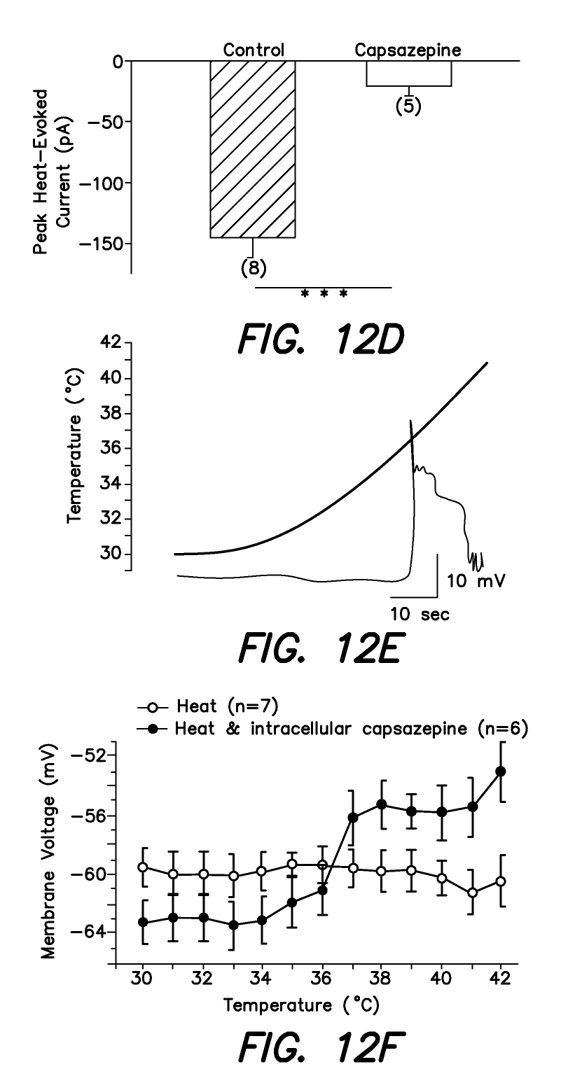

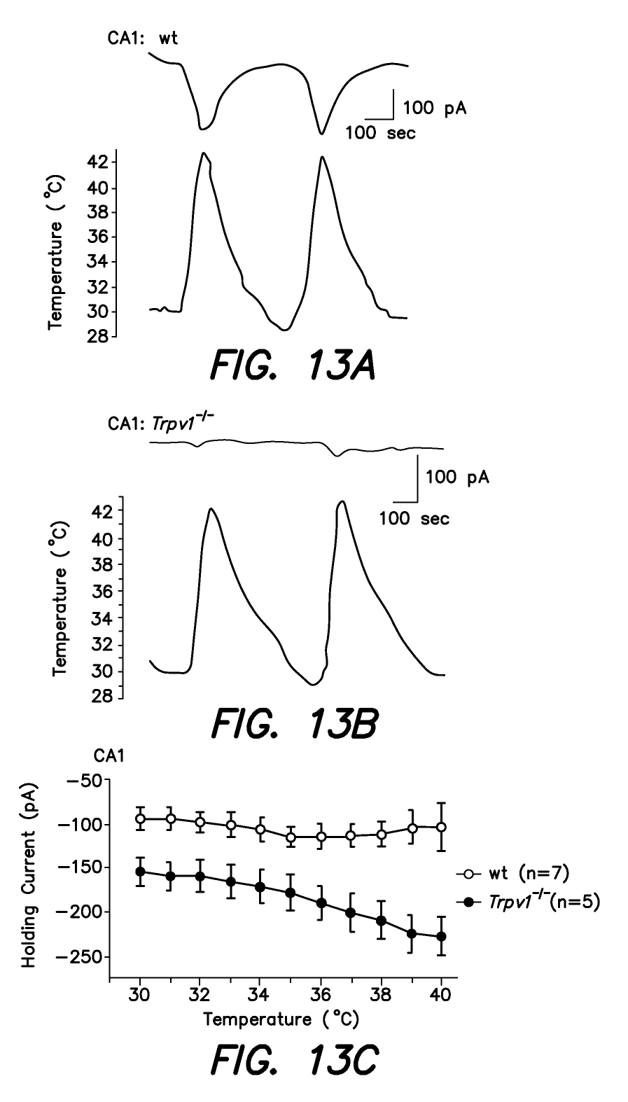

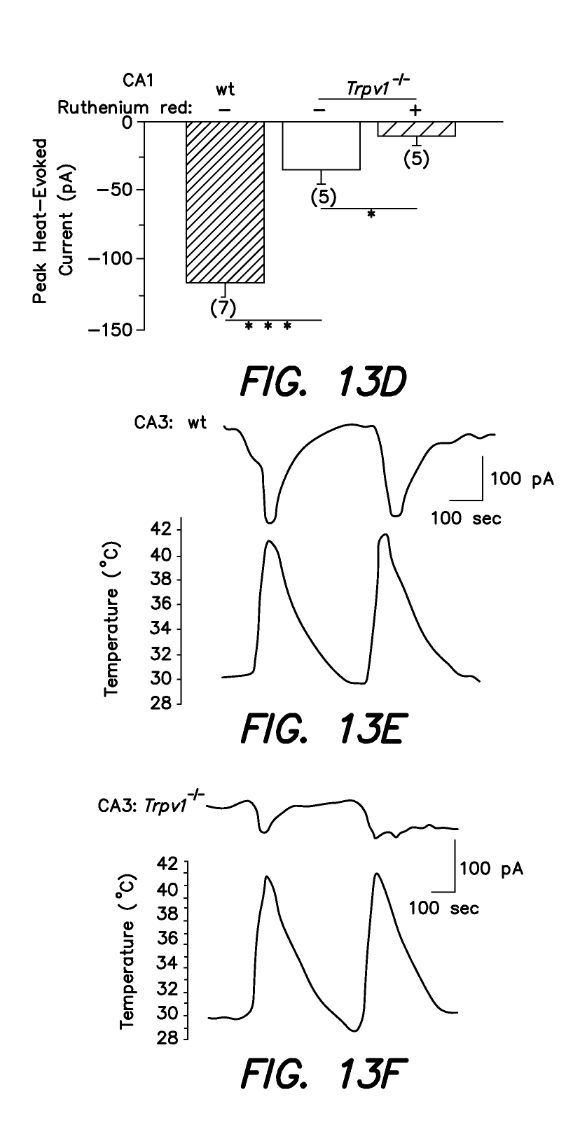

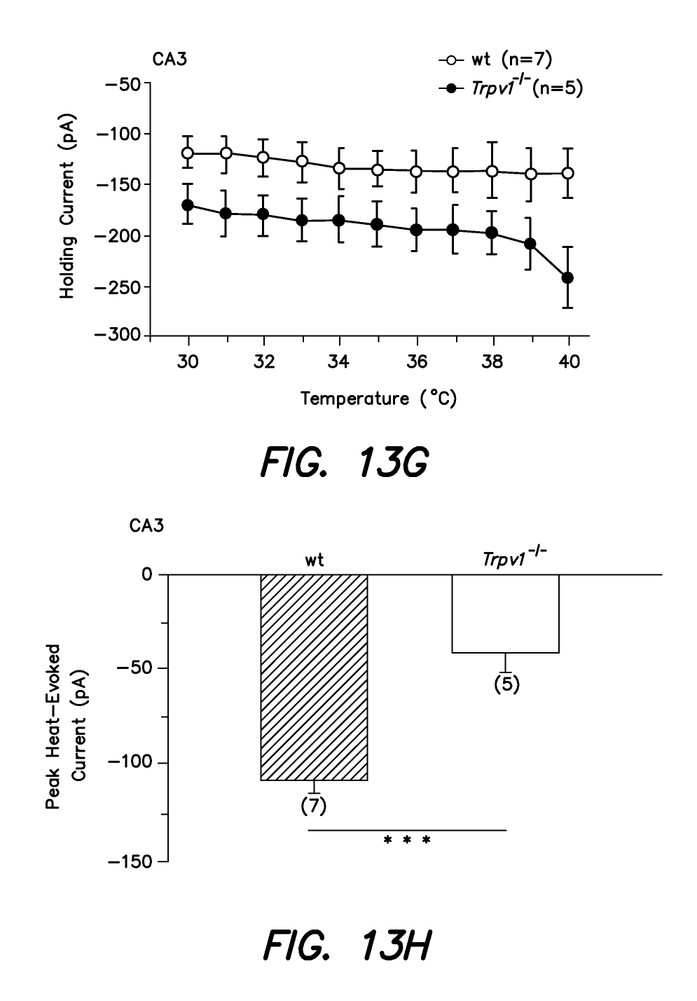

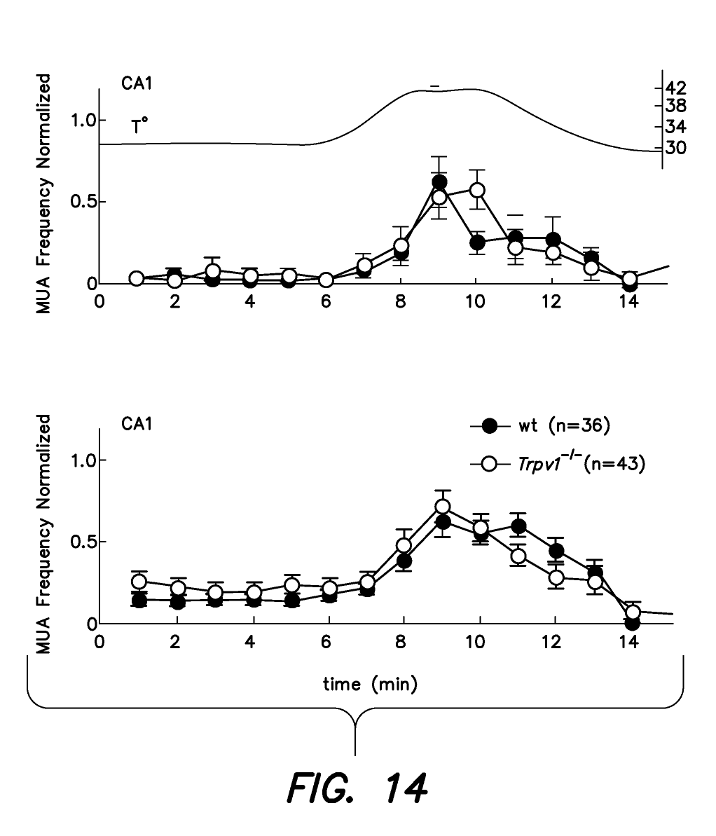

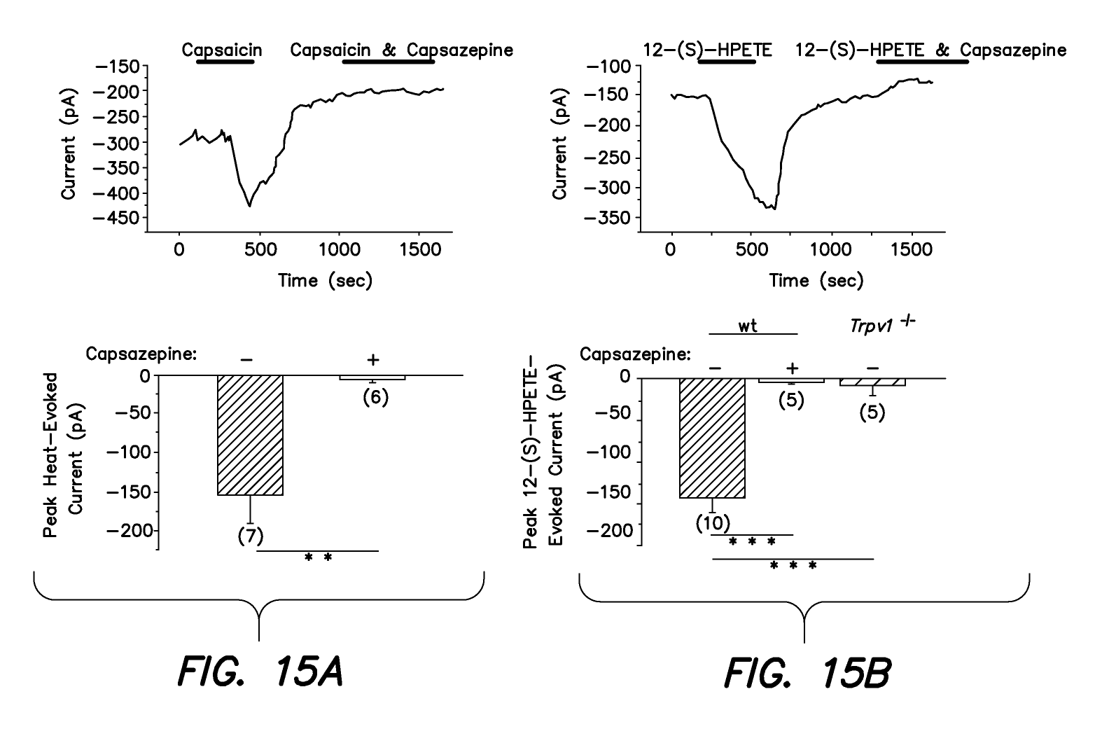

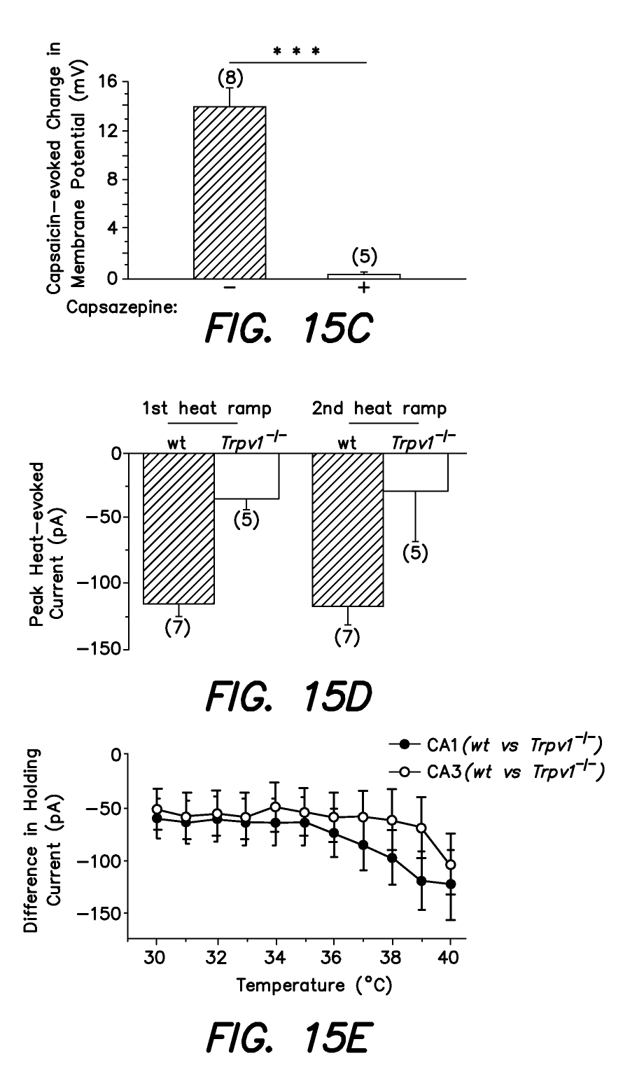

How might activation of TRPV1 channels increase the intrinsic excitability of hippocampal neurons? TRPV1 channels are nonselective cation channels, and their activation therefore triggers an inward current in cells that express them (Caterina et al., 2000). We made whole-cell recordings from acutely prepared brain slices from wild type C57BL/6 mice while blocking synaptic transmission and voltage-gated Na⁺ and Ca2+ channels. CA1 pyramidal neurons were recorded from while ramping the temperature from 30 to 42° C. over a period of 2 min (0.1° C./s). In voltage-clamp recordings, a substantial temperature-sensitive inward current was elicited above 31° C. (FIG. 12a); as expected for a TRPV1-mediated response, this current was largely eliminated by bath application of the TRPV1 antagonist capsazepine (FIG. 12b-d). In current-clamp recordings, temperature ramps markedly depolarized CA1 pyramidal neurons (FIG. 12e-f), and inclusion of 2 μM capsazepine in the intracellular pipette solution prevented heat-induced depolarization (FIG. 12f). Inward currents similar to those elicited with heat ramps were also observed using the TRPV1 channel agonists capsaicin and 12-(S)-HPETE (FIG. 15a-b). In current-clamp recordings, capsaicin application significantly depolarized CA1 pyramidal neurons, and this depolarization was prevented by the presence of 2 μM intracellular capsazepine (p<0.001; FIG. 15c). These results provide strong pharmacological evidence that TRPV1 channels underlie the thermosensitivity of CA1 pyramidal neurons.

If TRPV1 channels are required for the thermal sensitivity of hippocampal pyramidal neurons, then cells from trpv1^{-/-} mice should be relatively heat-insensitive. Consistent with this prediction, heat-induced currents were considerably reduced in CA1 pyramidal neurons from trpv1^{-/-} mice compared to those from wild-type mice (FIG. 13a-d). Furthermore, consecutive heat ramps induced reproducible inward currents and recovery in the majority of cells tested; this indicates that little sensitization or desensitization of current responses occurs over this time course (FIG. 15d). Residual heat-activated current in trpv1^{-/-} CA1 pyramidal neurons was reduced by bath application of ruthenium red, suggesting that other subtypes of ruthenium red-sensitive channels (perhaps other TRP channels) contribute to the thermosensitivity of CA1 neurons (FIG. 13d). Our data also revealed a difference between holding current values in wild-type vs. trpv1^{-/-} mice in both CA1 and CA3 pyramidal cells, suggesting that even at normal body temperature, there may be a standing TRPV1 channel conductance in these neurons (FIG. 13c,g; see also Shibasaki et al., 2007). These data confirm that TRPV1 channel activation is an integral component of heat-activated currents in CA1 pyramidal cells, and that the absence of TRPV1 channels diminishes thermal sensitivity.

The experiments illustrated in FIG. 11 suggested that heat-triggered neuronal bursting was most dramatically influenced by TRPV1 channels in hippocampal area CA3. We therefore next investigated whether TRPV1 receptor activation increased excitability as much in CA3 pyramidal cells as in CA1 pyramidal cells. Heat ramps evoked inward currents in wild-type CA3 cells (FIG. 13e), while responses in neurons from trpv1^{-/-} mice were attenuated (FIG. 13f-h). Thus, activation of TRPV1 channels by heat increases excitability in both hippocampal subregions. However, the current-temperature relationships for CA3 pyramidal cells in both wild-type and trpv1^{-/-} mice differ from those of CA1 pyramidal cells (p<0.01; FIG. 15e), suggesting that neurons from the two hippocampal subregions may either have TRPV1 channels with different subunit compositions or distinct modulatory states (Vellani et al., 2001; Huang et al., 2006; Cheng et al., 2007), or both. Cloned TRPV1 channels and those found in peripheral thermosensitive neurons are activated only at relatively high temperatures (>42° C.), while in hippocampal neurons temperature ramps elicit TRPV1 channel-dependent inward currents at temperatures that could be achieved in the brain during fever. Similar temperature thresholds have recently been reported for TRPV1 channels in hypothalamic neurons (Sharif-Naeini et al., 2008), as well as in peripheral neurons under specific conditions (Premkumar & Ahern, 2000). In the brain, where large temperature increases would be dangerous, TRPV1 channels may exist in a constitutively modulated state permitting channel activation at physiological temperatures.

Together, our results demonstrate that TRPV1 channels contribute to brain excitability. The expression of TRPV1 channels significantly reduces the temperature threshold and onset latency of hyperthermic seizures in mouse pups. Furthermore, in the principal cells of the hippocampus, TRPV1 channels mediate a direct, intrinsic, inward current that can be activated by heat. This TRPV1-mediated excitatory current may contribute to the febrile seizure susceptibility of the immature brain. There may be multiple mechanisms by which TRPV1 channels reduce febrile seizure thresholds. For example, our earlier work suggests that synaptic control of inhibitory hippocampal neurons is also regulated by TRPV1 channels (Gibson et al., 2008). Heat-induced seizures are not abolished in the absence of TRPV1 channels, so it is likely that other temperature-sensitive mechanisms also contribute to febrile seizures. TRPV1 channels are modulated by a variety of endogenous signaling molecules, including endocannabinoids, eicosanoids, and protein kinases (Kauer & Gibson, in press; Vellani et al., 2001; Huang et al., 2006; Bhave et al., 2003) whose expression levels can be altered under febrile conditions. Given that these channels play a critical role in setting the temperature threshold for seizures, the modulation state of TRPV1 channels may be an important determinant of febrile seizure susceptibility.

### Methods

All procedures were approved by the Brown University Animal Care and Use Committee. trpv1^{-/-} mice generated by Caterina et al. (2000) were obtained from Jackson Laboratories. Mice were maintained as a heterozygous breeding colony after back-crossing with C57BL/6 wild-type mice (Charles River Inc.)., and genotypes were determined by PCR. Experimenters were blind to the genotype until after data analysis.

The febrile seizure paradigm was conducted as described for mice (Baram et al., 1997; Dube & Baram, 2005). Briefly, at postnatal days 14-15, mice were placed in a glass container and hyperthermia was induced to 42° C. using a regulated stream of heated air. Rectal temperatures were measured at baseline and experimental hyperthermic seizure onset. The behavioral end-point of sudden immobility was previously reported to correlate with the onset of electroencephalogram seizures (Dube & Baram, 2005). Time of seizure onset was also recorded.

Coronal brain slices, 300-350 μm, were obtained from mice aged postnatal day 14-20 as previously described (Gibson et al., 2008; Gibson et al., 1999; McMahon & Kauer, 1997). Bathing perfusate was identical to previous except where indicated. A Haas-type interface chamber was used for field-potential recordings. Glass micropipettes were filled with 0.9% NaCl (resistance 400-700 kΩ). Simultaneous recordings were made in stratum pyramidale of CA1 and CA3 of each slice. Activity was recorded at 30° C. (5 min), ramped to 42° C. (5 min) and returned to 30° C. (10 min). Signals were amplified, digitally recorded at 10 kHz and stored (LabView).

CA1 and CA3 pyramidal whole-cell recordings were conducted as previously described (Gibson et al., 2008), with modifications as follows. Slices were held in a submerged chamber and perfused at 30° C. with (in mM): 119 NaCl, 26 NaHCO₃, 2.5 KCl, 1.0 NaH₂PO₄, 4.0 CaCl₂, 4.0 MgCl₂, 11 dextrose, 0.1 picrotoxin, 10 kynurenic acid, 2 CoCl₂, and 0.001 tetrodotoxin. Capsazepine (2 μM) was added to the intracellular pipette solution where indicated. Potassium gluconate replaced cesium gluconate in current-clamp experiments. Pyramidal cell holding current, input resistance and series resistance were measured in voltage-clamp at −65 mV; effects on membrane voltage were measured in current-clamp mode. Perfusate temperature was increased from 30° C. to 42° C. over 120 sec. Changes in holding current and temperature were measured simultaneously. Perfusate temperature for in vitro experiments was regulated by an in-line heater, temperature controller and bath thermistor (Warner Instruments).

For field-potential recordings, coronal slices, 350 μm thick, were obtained from P14-15 mice as previously described (Gibson et al., 2008; Gibson et al., 1999; McMahon & Kauer, 1997). Slices were kept at room temperature for at least 30 min until transferred to a 30° C. recording chamber. Artificial cerebrospinal fluid (ACSF) contained (in mM): 126 NaCl, 3 KCl, 1.25 NaH₂PO₄, 2 MgSO₄, 26 NaHCO₃, 10 dextrose and 2 CaCl₂, saturated with 95% O₂/5% CO₂. Glass micropipettes were filled with 0.9% NaCl (resistance 400-700 kΩ). Simultaneous recordings were made in stratum pyramidale of CA1 and CA3. For all field-potential recordings, activity was recorded at 30° C. (5 min), ramped to 42° C. (5 min) and returned to 30° C. (10 min). Signals were amplified, digitally recorded at 10 kHz and stored using an in-house LabView based program.

For hippocampal whole-cell recordings, 300 μm thick coronal slices were prepared from P14-P20 mice, transferred to a submerged recording chamber, and continuously perfused with ACSF warmed to 30° C. (except in experiments ramping temperature) at a flow rate of 1-2 ml/min. Oxygenated ACSF contained in mM: 119 NaCl, 26 NaHCO₃, 2.5 KCl, 1.0 NaH₂PO₄, 4.0 CaCl₂, 4.0 MgCl₂ and 11 dextrose, saturated with 95% O₂/5% CO₂ (pH 7.4). Picrotoxin (100 μM) and kynurenic acid (10 mM) were added to block GABAergic- and glutamatergic receptor-mediated synaptic transmission. In addition, for temperature ramp experiments, CoCl₂ (2 mM) and tetrodotoxin (1 μM) were used to block voltage-gated Ca²⁺ and Na channels, respectively.

Whole-cell patch clamp recordings were made from visually identified CA1 and CA3 pyramidal neurons. Patch pipettes contained (in mM): 117 cesium gluconate, 2.8 NaCl, 5 MgCl₂, 20 HEPES, 2 ATP-Na⁺, 0.3 GTP-Na⁺ and 0.6 EGTA. To record holding current, neurons were voltage-clamped at −65 mV (membrane potentials were not corrected for the liquid junction potential, estimated at approximately 10 mV). Effects on membrane voltage were measured in current-clamp mode with potassium gluconate substituted for cesium gluconate. All recordings were low-pass filtered at 3 kHz and sampled at 30 kHz (Digidata 1440A & pCLAMP software, Molecular Devices). The cell input resistance and series resistance were monitored throughout and cells were discarded if these values changed by more than 10%. To determine the temperature responses of hippocampal pyramidal cells, the perfusate temperature was increased from 30° C. to approximately 42° C. over 120 sec (0.1° C./s) while recording either membrane potential or holding current. The peak change in holding current was measured simultaneously with the temperature monitored at the local thermistor probe tip. As noted, in some experiments, 2 μM capsazepine was included in the intracellular patch pipette solution.

Perfusate temperature for all in vitro experiments was regulated by either a TC-324A or TC-344B temperature controller and an in-line heater (Warner Instruments). The actual temperature of the perfusate was monitored via thermistor probes placed near the slice. For whole-cell recordings, the temperature of the bath was additionally maintained by a PM-1 platform heater (Warner Instruments). To further facilitate rapid temperature changes, patch-clamp recordings were performed in a small volume (0.12 ml) recording chamber, bath volume was reduced as much as possible, and the microscope objective was removed from the bath.

Receptor antagonists were added directly to the ACSF at known concentrations for at least 15 min prior to temperature ramps. Control experiments were interleaved with experiments using bath-applied receptor antagonists or involving slices from trpv1^{-/-} mice. To assess drug and temperature effects, the magnitude of holding current (voltage-clamp) or membrane voltage (current-clamp) were calculated and averaged for a 1 min time period during the peak drug or temperature response and compared to the magnitude of averaged data during a 1 min time period immediately prior to drug or temperature application.

Multi-unit activity (MUA) was high-pass filtered at 500 Hz and spikes were detected using threshold-crossing criteria. Frequency, in Hz, was averaged per min. Mean frequencies within each experiment were then normalized to the maximum mean frequency found between 0 to 3 min into the cooling period after the maximum temperature was reached.

All combined data are expressed as mean±the standard error of the mean (s.e.m.). Comparisons of the means observed in different experimental groups, for the behavioral assay and all whole-cell recordings, were performed using a t-test (unpaired, two-tailed, with Welch's correction if the variances between the groups were unequal) or analysis of variance (ANOVA) repeated measures analysis with a post-hoc Dunnett's test as appropriate (GraphPad Prism, Version 4). Linear regression analysis was used to determine current-temperature relationships. Incidence rates of increased MUA activity in response to heat were compared using one-tailed Fisher's exact test. Significance was defined as p<0.05. The n values reported refer to the number of slices.

12-(S)-HPETE [12-(S)-Hydroperoxyeicosa-5Z, 8Z, 10E, 14Z-tetraenoic acid] was purchased from Biomol International. Capsaicin, capsazepine, ruthenium red and tetrodotoxin citrate were obtained from Tocris Bioscience. All other chemicals were purchased from Sigma-Aldrich. Capsaicin and capsazepine were dissolved in DMSO and then diluted at least 1:1000 to the final concentration in ACSF, or for intracellularly applied capsazepine, at least 1:5000 to the final concentration in the intracellular patch pipette solution. The responses of neurons to 0.1% DMSO were tested in our preliminary experiments, and no detectable effect was found.

### References for Example 2

Holtzman, D., Obana, K. & Olson, J. Hyperthermia-induced seizures in the rat pup: a model for febrile convulsions in children. Science 213, 1034-1036 (1981).
Tancredi, V., et al. Induction of epileptiform activity by temperature elevation in hippocampal slices from young rats: an in vitro model for febrile seizures? Epilepsia 33, 228-234 (1992).
Baram, T. Z., Gerth, A. & Schultz, L. Febrile seizures: an appropriate-aged model suitable for long-term studies. Brain Res Dev Brain Res 98, 265-270 (1997).
Schuchmann, S., et al. Experimental febrile seizures are precipitated by a hyperthermia-induced respiratory alkalosis. Nat Med 12, 817-823 (2006).
Caterina, M. J., et al. Impaired nociception and pain sensation in mice lacking the capsaicin receptor. Science 288, 306-313 (2000).
Dhaka, A., Viswanath, V. & Patapoutian, A. TRP ion channels and temperature sensation. Annu Rev Neurosci 29, 135-161 (2006).
Gibson, H. E., et al. TRPV1 channels mediate long-term depression at synapses on hippocampal interneurons. Neuron 57, 746-759 (2008).
Shibasaki, K., Suzuki, M., Mizuno, A. & Tominaga, M. Effects of body temperature on neural activity in the hippocampus: regulation of resting membrane potentials by transient receptor potential vanilloid 4. J Neurosci 27, 1566-1575 (2007).
Lipski, J., et al. Involvement of TRP-like channels in the acute ischemic response of hippocampal CA1 neurons in brain slices. Brain Res 1077, 187-199 (2006).
Huang, S. M., et al. An endogenous capsaicin-like substance with high potency at recombinant and native vanilloid VR1 receptors. Proc Natl Acad Sci USA 99, 8400-8405 (2002).
Al-Hayani, A., et al. The endogenous cannabinoid anandamide activates vanilloid receptors in the rat hippocampal slice. Neuropharmacology 41, 1000-1005 (2001).
Hajos, N. & Freund, T. F. Distinct cannabinoid sensitive receptors regulate hippocampal excitation and inhibition. Chem Phys Lipids 121, 73-82 (2002).
Kauer, J. A. & Gibson, H. E. Hot Flash: TRPV channels in the brain. Trends Neurosci (in press).
Toth, A., et al. Expression and distribution of vanilloid receptor 1 (TRPV1) in the adult rat brain. Brain Res Mol Brain Res 135, 162-168 (2005).
Mitchell, T. V. & Lewis, D. V. in Febrile Seizures (eds Baram, T. Z. & Shinnar, S.) 103-126 (2002)
Spruston, N. & McBain, C. in The Hippocampus Book (eds Andersen, P., Morris, R., Amaral, D., Bliss, T. & O'Keefe, J.) 133-201 (2007)
Prince, D. A. & Connors, B. W. Mechanisms of interictal epileptogenesis. Adv Neurol 44, 275-299 (1986).
Vellani, V., et al. Protein kinase C activation potentiates gating of the vanilloid receptor VR1 by capsaicin, protons, heat and anandamide. J Physiol 534, 813-825 (2001).
Huang, J., Zhang, X. & McNaughton, P. A. Modulation of temperature-sensitive TRP channels. Semin Cell Dev Biol 17, 638-645 (2006).
Cheng, W., Yang, F., Takanishi, C. L. & Zheng, J. Thermosensitive TRPV channel subunits coassemble into heteromeric channels with intermediate conductance and gating properties. J Gen Physiol 129, 191-207 (2007).
Sharif-Naeini, R., Ciura, S. & Bourque, C. W. TRPV1 gene required for thermosensory transduction and anticipatory secretion from vasopressin neurons during hyperthermia. Neuron 58, 179-185 (2008).
Premkumar, L. S. & Ahern, G. P. Induction of vanilloid receptor channel activity by protein kinase C. Nature 408, 985-990 (2000).
Bhave, G., et al. Protein kinase C phosphorylation sensitizes but does not activate the capsaicin receptor transient receptor potential vanilloid 1 (TRPV1). Proc Natl Acad Sci USA 100, 12480-12485 (2003).
Dube, C. & Baram, T. Z. in Models of Seizures and Epilepsy (eds Pitkänen, A., Schwartzkroin, P. A. & Moshe, S. L.) 333-341 (2005).
Gibson, J. R., Beierlein, M. & Connors, B. W. Two networks of electrically coupled inhibitory neurons in neocortex. Nature 402, 75-79 (1999).
McMahon, L. L. & Kauer, J. A. Hippocampal interneurons express a novel form of synaptic plasticity. Neuron 18, 295-305 (1997).

### Equivalents

The foregoing written specification is considered to be sufficient to enable one ordinarily skilled in the art to practice the invention. The present invention is not to be limited in scope by examples provided, since the examples are intended as mere illustrations of one or more aspects of the invention. Other functionally equivalent embodiments are considered within the scope of the invention. Various modifications of the invention in addition to those shown and described herein will become apparent to those skilled in the art from the foregoing description. Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth or illustrated in the drawings. The invention is capable of other embodiments and of being practiced or of being carried out in various ways.

Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

All references, patents and patent applications that are recited in this application are incorporated by reference herein in their entirety.

1. A method for treatment or prophylaxis of epilepsy comprising administering to a subject having epilepsy, suspected of having epilepsy or at risk of developing epilepsy an amount of a TRPV1 antagonist effective to reduce epileptic seizures or prevent the onset of epileptic seizures.

2. The method of claim 1, wherein the TRPV1 antagonist is capsazepine, SR141716A, or 5′-Iodoresiniferatoxin.

3. The method of claim 1, wherein the TRPV1 antagonist is administered orally, sublingually, buccally, intranasally, intravenously, intramuscularly, intrathecally, intraperitoneally, or subcutaneously.

4. A method for treatment or prophylaxis of epilepsy comprising
administering to a subject having epilepsy, suspected of having epilepsy or at risk of developing epilepsy an amount of a TRPV1 agonist effective to reduce epileptic seizures or prevent the onset of epileptic seizures.

5. The method of claim 4, wherein the TRPV1 agonist is resiniferatoxin, tinyatoxin, anandamide, capsaicin or a capsaicinoid.

6. The method of claim 4, wherein the TRPV1 agonist is administered orally, sublingually, buccally, intranasally, intravenously, intramuscularly, intrathecally, intraperitoneally, or subcutaneously.

7. A method for treatment or prophylaxis of epilepsy comprising
administering to a subject having epilepsy, suspected of having epilepsy or at risk of developing epilepsy an amount of a molecule that reduces the expression of TRPV1 effective to reduce epileptic seizures or prevent the onset of epileptic seizures.

8. The method of claim 7, wherein the molecule that reduces the expression of TRPV1 is molecule that produces RNA interference.

9. The method of claim 8, wherein the molecule that produces RNA interference is a siRNA molecule or a shRNA molecule.

10. The method of claim 7, wherein the molecule that reduces the expression of TRPV1 is administered orally, sublingually, buccally, intranasally, intravenously, intramuscularly, intrathecally, intraperitoneally, or subcutaneously.

11. A method for treatment or prophylaxis of febrile seizures comprising
administering to a subject having a febrile seizure, suspected of having a febrile seizure or at risk of developing a febrile seizure an amount of a TRPV1 antagonist effective to reduce the febrile seizure or prevent the onset of the febrile seizure.

12. The method of claim 11, wherein the TRPV1 antagonist is capsazepine, SR141716A, or 5′-Iodoresiniferatoxin.

13. The method of claim 11, wherein the TRPV1 antagonist is administered orally, sublingually, buccally, intranasally, intravenously, intramuscularly, intrathecally, intraperitoneally, or subcutaneously.

14. A method for treatment or prophylaxis of febrile seizures comprising
administering to a subject having a febrile seizure, suspected of having a febrile seizure or at risk of developing a febrile seizure an amount of a TRPV1 agonist effective to reduce the febrile seizure or prevent the onset of the febrile seizure.

15. The method of claim 14, wherein the TRPV1 agonist is resiniferatoxin, tinyatoxin, anandamide, capsaicin or a capsaicinoid.

16. The method of claim 14, wherein the TRPV1 agonist is administered orally, sublingually, buccally, intranasally, intravenously, intramuscularly, intrathecally, intraperitoneally, or subcutaneously.

17. A method for treatment or prophylaxis of febrile seizures comprising
administering to a subject having a febrile seizure, suspected of having a febrile seizure or at risk of developing a febrile seizure an amount of a molecule that reduces the expression of TRPV1 effective to reduce the febrile seizure or prevent the onset of the febrile seizures.

18. The method of claim 17, wherein the molecule that reduces the expression of TRPV1 is molecule that produces RNA interference.

19. The method of claim 18, wherein the molecule that produces RNA interference is a siRNA molecule or a shRNA molecule.

20. The method of claim 17, wherein the molecule that reduces the expression of TRPV1 is administered orally, sublingually, buccally, intranasally, intravenously, intramuscularly, intrathecally, intraperitoneally, or subcutaneously.
